(19) United States
(12) Patent Application Publication
(10) Pub. No.: US 2018/0073018 A1
(43) Pub. Date: Mar. 15, 2018

(54) COMPOSITIONS AND METHODS FOR SPECIFIC REACTIVATION OF HIV LATENT RESERVOIR

(71) Applicant: Temple University - of the Commonwealth System of Higher Education, Philadelphia, PA (US)

(72) Inventors: Wenhui Hu, Cherry Hill, NJ (US); Kamel Khalili, Bala Cynwyd, PA (US); Yonggang Zhang, Maple Shade, NJ (US)

(21) Appl. No.: 15/705,825

(22) Filed: Sep. 15, 2017

Related U.S. Application Data
(63) Continuation of application No. PCT/US2016/022854, filed on Mar. 17, 2016.
(60) Provisional application No. 62/134,231, filed on Mar. 17, 2015.
(60) Provisional application No. 62/242,774, filed on Oct. 16, 2015.

(51) Int. Cl. C12N 15/11; A61K 48/00; C12N 7/00; C12N 9/22; C12N 15/113; C07K 14/005

(52) U.S. Cl. CPC C12N 15/11; A61K 48/005; C12N 7/00; C12N 9/22; C12N 15/1132; C12N 2795/18122; C12N 2310/20; C12N 2740/16022; C12N 2740/16051; C12N 2740/16062; C12N 2310/16; C07K 14/005

(57) ABSTRACT

The present invention relates to compositions and methods for reactivation of HIV in vivo or in vitro. latently infected cell. In one embodiment, the present invention uses the CRISPR/Cas9 system in combination with the MS2 bacteriophage coat protein-mediated synergistic activation mediator (SAM) system to provide enhanced transcriptional activation of the HIV genome.

### CROSS REFERENCE TO RELATED APPLICATIONS

This application claims the benefit of filing dates of U.S. Provisional Application U.S. Ser. No. 62/134,231 filed Mar. 17, 2015 and U.S. Provisional Application U.S. Ser. No. 62/242,774 filed Oct. 16, 2015, the complete disclosures of which are herein incorporated by reference.

### BACKGROUND

Infection with HIV-1 remains a major public health problem affecting more than 35 million people worldwide and more than 1.2 million people in the United States. Combined antiretroviral therapy (cART) can achieve a “functional cure”, but HIV-1 resurgence in latently infected cells after cART withdrawal is a main obstacle to a permanent cure of HIV-1 infection. Current cART does not eliminate the integrated and transcriptionally silent HIV-1 provirus in latently infected cells. While the introduction of combined antiretroviral therapy (cART) has greatly improved survival rates among AIDS patients, a substantial portion of HIV-1 infected individuals remain at risk for the development of AIDS as a result of reactivation of latently infected cells, partly due to non-adherence to medication and emergence of drug resistant viruses.

Moreover, HIV-1 positive long term survivors continue to develop comorbidities including an accelerated aging process, neurocognitive disorders, heart failure, and others. Gradual reactivation of the integrated HIV-1 genome in latently infected cells can result in superactivation of the HIV-1 long-term repeats (LTR) and the initiation of the productive infection cycle.

Current antiretroviral therapy does not eliminate the integrated and transcriptionally silent HIV provirus in latently infected cells. A “shock and kill” (also called “kick and kill” or “reactivation and elimination”) strategy to eradicate HIV latent reservoir has been becoming very promising, wherein reactivation of latent proviruses allows clearance of latent cells by viral cytotoxicity and/or host immune defense. The concomitant antiretroviral treatment will prevent virus spread and block new infection (Sgarbanti and Battistini, 2014, Curr Opin Virol 3:394-401). Several reagents or small molecules, in particular, the HDAC inhibitors, have been developed to reactivate HIV latent reservoir, some of which have been used in clinical trials (Wei et al., 2014, PLoS Pathog 10:e1004071; Lucera et al., 2014, J Virol 88:10803-12; Spivak et al., Clin Infect Dis 58:883-90; Xing and Silicano, 2013, Drug Discov Today 18:541-51). However, the reactivation results are disappointing, likely due to insufficient reactivation, non-specific cell targeting and drug toxicity (Lucera et al., 2014, J Virol 88:10803-12; Spivak et al., Clin Infect Dis 58:883-90; White et al., 2015, Antiviral Res 123:78-85). For example, a recent report using a humanized HIV-1 latency mouse model demonstrated that only combined treatment with three well-established latency-reversing agents including the histone deacetylase inhibitor vorinostat (suberoylanilide hydroxamic acid, SAHA), the BET bromodomain protein inhibitor I-BET 151, and the immune modulatory anti-CTLA4 antibody, allows HIV upregulation to a sufficient level in HIV latent reservoir cells for the elimination by the broadly neutralizing anti-HIV antibodies (Halper-Stromberg et al., 2014, Cell 158:989-99). Multiple activating, latency-reversing agents at several signal pathways (Laird et al., 2015, J Clin Invest 125:1901-12) will definitely increase the toxicity to HIV-negative cells, similar to the chemotherapies for treatment of cancerous cells. Furthermore, repeated administration of these latency-reversing agents is required to maintain a continuous reactivation of HIV latent reservoir. Therefore, a better reactivator of latent HIV virus, which displays targeted cell specificity, sustained high efficiency and no/low cytotoxicity remains to be identified. To achieve HIV-targeted specific reactivation, ZFN and TALEN have been tested by engineering target-specific transcriptional activators such as VP64 (Wang et al., 2014, Gene Ther 21:490-5; Wang et al., 2015, AIDS Res Hum Retroviruses 31:98-106), but the reported efficiency was marginal (1-2 folds).

So far, there have been no reports on catalytically-deficient Cas9 (dCas9)-mediated HIV-1 reactivation, particularly the dCas9-synergistic activation mediator (dCas9-SAM) system (Konermann et al., 2015, Nature 517:583-8), although a recent review discussed its plausibility as an effective HIV-1 therapy (Saayman et al., 2015, Expert Opin Biol Ther 15:819-30).

Successful application of CRISPR/Cas9 technology to mammalian system for genome editing was first reported in early 2013 (Cong et al., 2013, Science 339:819-23; Mali et al., 2013, Science 339:823-6). Since then, this novel genome editing system has attracted a huge amount of attention in biomedical field, and subsequent examples of this system's effectiveness have been seen in the fields of animal models, genetic diseases, cancer biology and infectious diseases (Saayman et al., 2015, Expert Opin Biol Ther 15:819-30; Sander and Joung et al., 2014, Nat Biotechnol 32:347-55; Vasileva et al., 2015, Cell Deth Dis 6:e1831; Sanchez-Rivera and Jacks, 2015, Nat Rev Cancer 15:387-95; Riordan et al., 2015, Cell & Biosci 5:33). Simultaneously, the use of dCas9 conjugated with a single transcriptional activator or repressor to manipulate cellular gene regulation has been developed (Agne et al., 2014, ACS Synth Biol 3:986-9; Maeder et al., 2013, Nat Methods, 10:977-9; Gilbert et al., 2013, Cell 154:422-51; Cheng et al., 2013, Cell Res 23:1163-71). However, this single regulator system has its limitations, such as effectiveness of gene activation/repression and scalability.

### SUMMARY

Embodiments of the invention are directed to, inter alia, a composition for reactivation of a retrovirus in vitro or in vivo comprising: an isolated nucleic acid encoding a guide nucleic acid, wherein the guide nucleic acid comprises a targeting nucleotide sequence directed to one or more target sequences in the retroviral genome; an isolated nucleic acid encoding a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

In certain embodiments, the CRISPR/Cas fusion protein comprises catalytically deficient Cas protein (dCas), orthologs, homologs, mutants variants or fragments thereof, fused with one or more transcriptional activators.

In an embodiment, the RNA binding protein, fragments, mutants, derivatives or variants thereof, comprises a bacteriophage coat protein.

In another embodiment, the one or more transcriptional activators comprise VP64, p65, HSF1, p65AD, Rta, Sp1, Vax, GATA4, fragments, mutants, or any combinations thereof.

In another embodiment, the one or more target sequences comprise a nucleic acid sequence having at least about 75% sequence similarity to any one or more sequences comprising SEQ ID NOS: 1-152 or combinations thereof.

In yet another embodiment, the one or more target sequences comprise one or more nucleic acid sequences comprising SEQ ID NOS: 1-152 or combinations thereof.

In certain embodiments, an isolated nucleic acid encoding: a guide nucleic acid, wherein the guide nucleic acid comprises a targeting nucleotide sequence directed to one or more target sequences in the retroviral genome; a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

In other embodiments, an isolated nucleic acid molecule encoding at least one guide nucleic acid molecule (gRNA), an isolated nucleic acid encoding a Clustered Regularly Interspaced Short Palindromic Repeat (CRISPR)-associated endonuclease/Cas (CRISPR/Cas) fusion protein or functional fragment or derivative thereof, and an isolated nucleic acid encoding an RNA binding protein, functional fragments, mutants, derivatives or variants thereof.

In other embodiments, a composition for reactivation of HIV in a latently infected cell, in vivo or in vitro, comprises: a) one selected from the group consisting of an isolated guide nucleic acid and an isolated nucleic acid encoding a guide nucleic acid, wherein the guide nucleic acid comprises a targeting nucleotide sequence directed to a target sequence in the HIV genome; b) one selected from the group consisting of a catalytically deficient Cas9 (dCas9) fusion protein, and an isolated nucleic acid encoding a dCas9 fusion protein, wherein the dCas9 fusion protein comprises dCas9 and one or more transcriptional activators; and, c) one selected from the group consisting of a MS2 fusion protein, and an isolated nucleic acid encoding a MS2 fusion protein, wherein the MS2 fusion protein comprises MS2 bacteriophage coat protein and one or more transcription activators.

In certain embodiments, the target sequence comprises a sequence within HIV LTR.

In other embodiments, the target sequence comprises one or more nucleic acid sequences comprising SEQ ID NOS: 1-152 or combinations thereof. In yet another embodiment, the target sequence is selected from the group consisting of SEQ ID NO: 94 (LTR-J), SEQ ID NO: 96 (LTR-L), SEQ ID NO: 98 (LTR-N) and SEQ ID NO: 99 (LTR-O).

In other embodiments, the guide nucleic acid comprises a hairpin aptamer capable of binding to MS2.

In certain embodiments, the composition comprises one or more isolated nucleic acids, where the one or more isolated nucleic acids encode multiple guide nucleic acids, wherein each guide nucleic acid comprises a targeting nucleotide sequence directed to a different target sequence in the HIV genome.

Other aspects are described infra.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A, FIG. 1B, and FIG. 1C, depict results of experiments comprising HIV-1 LTR-gRNA screening for dCas9-VP64-induced reactivation of latent LTR-luciferase reporter in TZM-bI cells. FIG. 1A is a schematic illustration depicting HIV-gRNA target sites. FIG. 1B is a schematic depicting gRNA/dCas9-VP64 binding to a target region of HIV-LTR that drives luciferase expression in TZM-bI cells. FIG. 1C depicts the results of experiments investigating the reactivation of HIV-LTR (as measured by luciferase reporter) induced by the various gRNAs. The TZM-bI cells were plated in 96-well plate and the indicated pKLV-gRNA-Puro-2A-BFP lentiviral vectors were cotransfected in quadruplicates by Lipofectamine 3000 with pMSCV-LTR-dCas9-VP64-BFP vector. After 3 days, the luminescence of the cell lysate was measured with OneGlo luminescence kit.

FIG. 2A, FIG. 2B, and FIG. 2C, depict results of experiments comprising HIV-1 LTR-MS2-gRNA screening for LTR reactivation by SAM complex dCas9-VP64/MS2-p65-HSF1 in HEK293T cells. FIG. 2A and FIG. 2B depict a schematic of complex formation at the target site, where the complex comprises MS2-binding gRNA, dCas9-VP64, and MS2-p65-HSF1. FIG. 2C is a graph depicting the results of experiments investigating the reactivation of HIV-LTR induced by the various MS2-binding gRNA using pEcoHIV-NL4-3-eLuc reporter assay.

FIG. 3A and FIG. 3B, depict the results of experiments demonstrating that gene delivery by either transient plasmid transfection (FIG. 3A) or stable lentivirus infection (FIG. 3B) induced similar reactivation of HIV-LTR-luciferase reporter in TZM-bI cells. Cells were plated in 96-well plate and the indicated MS2-gRNAs were cotransfected by Lipofectamine 3000 (FIG. 3A) or coinfected by lentiviruses (FIG. 3B, MOI=50) with pMSCV-LTR-dCas9-VP64-BFP plus pLV-MS2-p65-HSF1-GFP. Two days later, OneGlo luminescence assay was performed. The SIV MS2-gRNA-D was used as negative control. Note that MS2-p65-HSF1 alone did not influence the basal activity of LTR-luciferase reporter. The gRNA-J has no effect due to 2 nucleotides mismatch to the LTR sequence from TZM-bI cells.

FIG. 4 is a graph depicting the results of experiments which demonstrate that inducible and constitutive dCas9-VP64 expression induced similar activation of EcoHIV luciferase reporter in HEK293T cells. The indicated MS2-gRNAs were cotransfected with Tet-inducible pHAGE-TRE-dCas9-VP64 or constitutive pMSCV-LTR-dCas9-VP64-BFP plus pLV-MS2-p65-HSF1-GFP and pEcoHIV-NL4-3-eLuc. Two days later, OneGlo luminescence assay was performed.

FIG. 5A, FIG. 5B, and FIG. 5C, depict the results of experiments demonstrating that MS2-gRNA/SAM system induced robust and persistent reactivation of HIV-1 EGFP reporter in latent CHME5 microglial cells (FIG. 5A). Cells were infected with pMSCV-LTR-dCas9-VP64-puromycin and Lenti-MS2-p65-HSF1-hygromysin, selected for 10 days. The population cells were infected with Lenti-MS2-gRNAs-Zeosin and the fluorescent micrographs were taken at 1 day after infection (FIG. 5B). Cells were continuously cultured for 7 days, and selected with triple antibiotics (puromycin-hygromycin-Zeocin) for 3 days before flow cytometry (FIG. 5C).

FIG. 6 is a schematic illustration of the “shock and kill” strategy for eliminating latent HIV infection.

FIG. 7A, FIG. 7B, FIG. 7C, FIG. 7D, and FIG. 7E, depict the results of experiments of the screening of single guide RNAs (sgRNAs) that guide dCas9-transcriptional activator to the HIV-1 long terminal repeat (LTR) promoter. FIG. 7A is an illustration of sgRNA-guided HIV-1 genome regulation platform for dCas9 fusion with transcriptional activator (VP64). FIG. 7B is a schematic representation of dCas9-VP64 expressing lentiviral or retroviral vector and sgRNA expressing lentiviral vector (LV). FIG. 7C is a schematic illustration of sgRNA targeting locations within HIV-1 LTR. Some of transcription factors are shown in different colors. FIG. 7D is a graph showing the identification of effective sgRNAs with dCas9-VP64 in HEK293T cells transiently coexpressing NL4-3-EcoHIV-firefly luciferase (eLuc) reporter. FIG. 7E is a graph showing the identification of effective sgRNAs with dCas9-VP64 in TZM-bI cells integrated with LTR-eLuc reporter. At 3 days after cotransfection, ONE-Glo™ luciferase assay was performed. Data represent mean±SEM of 4 independent transfections, showing fold changes in luminescent reporter activity relative to corresponding empty sgRNA expression vector (LTR-0). *p<0.05 and **p<0.01 indicate statistical significance by ANOVA and FLSD test.

FIG. 8A, FIG. 8B, FIG. 8C, and FIG. 8D, depict the results of experiments of screening of MS2-mediated sgRNAs (msgRNAs) targeting the HIV-1 long terminal repeat (LTR) in the dCas9-synergistic activation mediator (SAM) system. FIG. 8A is a schematic illustration of SAM-VPH complex derived from dCas9-VP64 (V) and MS2-p65-HSF1 (PH) with msgRNAs. FIG. 8B is a graph showing similar activation of transfected EcoHIV-eLuc reporter by inducible (TRE) or constitutive (LTR) dCas9-VP64 expression in HEK293T cells. FIG. 8C is a graph showing similar reactivation of latent HIV-LTR-eLuc reporter in TZM-bI cells after either transient plasmid transfection by Lipofectamine 3000 or stable infection by lentivirus. Note that MS2-p65-HSF1 alone did not influence the basal activity of LTR-eLuc reporter. The gRNA-J has no effect due to 2 nucleotides mismatch to the LTR sequence from TZM-bI cells. FIG. 8D is a graph showing the identification of msgRNA LTR-L and O with best efficiency to activate EcoHIV-eLuc reporter in HEK239T cells with dCas9-SAM system. Data represent mean±SEM of 4-6 independent transfections or infections, showing fold changes relative to corresponding empty msgRNA expression vector (LTR-0). *p<0.05 and **p<0.01 indicate statistical significance by ANOVA and FLSD test.

FIG. 9A, FIG. 9B, FIG. 9C, FIG. 9D, FIG. 9E, and FIG. 9F, depict the results of experiments demonstrating robust reactivation of HIV-1 LTR promoter by lentivirus-mediated dCas9-SAM in HIV-1 latent cell lines. FIG. 9A and FIG. 9B: TZM-bI cells were infected with dCas9-VPH and indicated msgRNA lentiviruses at 1 or 10 MOI and luciferase activity was measured at 2-21 days post-infection (dpi). FIG. 9C, FIG. 9D: indicated HIV-1 EGFP reporter T cell lines were infected with dCas9-VPH and indicated msgRNAs lentiviruses at 10 MOI and flow cytometric analysis for EGFP percentage (FIG. 9C) and individual intensity (FIG. 9D) was performed at 1 dpi. FIG. 9E, FIG. 9F: HIV-1 latent CHME5 microglia cells were infected at 10 MOI and flow cytometry was performed at 1 dpi. All data represent relative changes (n=3-4) to the corresponding empty msgRNA control (Zero). *p<0.05 and **p<0.01 indicate statistical significance by ANOVA and FLSD test.

FIG. 10A, FIG. 10B, FIG. 10C, FIG. 10D, FIG. 10E, FIG. 10F, and FIG. 10G, depict the results of experiments demonstrating potent and persistent reactivation of HIV-1 LTR promoter induces suicide death of HIV-1 latent 2D10 T and CHME5 microglia cells but not TZM-bI cells due to the production of toxic viral proteins. FIG. 10A, FIG. 10B: TZM-bI cells were infected with indicated lentiviruses for 1 d and triple antibiotic selection with puromycin/hygromycin/zeoin (P/H/Z) was maintained for 4 d before passage, OneGlo luciferase reporter assay (FIG. 10A) and CellTiter-Glo® luminescent cell viability assay (FIG. 10B). FIG. 10C, FIG. 10D: CHME5 cells were infected with indicated lentiviruses and cultured for 5 d in the absence or presence of P/H/Z triple selection. Flow cytometry analysis was performed for reactivation efficiency (FIG. 10C) and total survival cell number (FIG. 10D). FIG. 10E: Caspase-Glo® 3/7 Assay was performed at 2 dpi to evaluate apoptotic cell death. The reactivation efficiency was validated by flow cytometry (EGFP % on the bottom). FIG. 10F, FIG. 10G: The 2D10 cells were infected for 1 and 4 dpi, and the reactivation efficiency (FIG. 10F) and relative total survival cell number (FIG. 10G) were determined by flow cytometry. *p<0.05 and **p<0.01 indicate statistical significance by ANOVA and FLSD test.

FIG. 11A, FIG. 11B, and FIG. 11C depict the results of experiments demonstrating potent and persistent reactivation of HIV-1 EGFP reporter virus and suicide cell death by dCas9-VPH/msgRNA in CHME5 microglia cells (FIG. 11A) and Jurkat-derived 2D10 (FIG. 11B) or E4 (FIG. 11C) T cell lines. FIG. 11A: Time-dependent reduction in EGFP+CHME5 cells due to continuous reactivation and suicide cell death. P/H/Z triple selection enriched lentivirus-infected cells and validated continuous EGFP reporter reactivation. **p<0.01 indicate statistical significance by student's t test as compared to corresponding non-selection groups. FIG. 11B, FIG. 11C: Time-dependent reduction in EGFP+2D10 or E4 cells due to continuous reactivation and suicide cell death.

FIG. 12A, FIG. 12B, FIG. 12C, FIG. 12D, FIG. 12E, FIG. 12F, and FIG. 12G, depict the results of experiments demonstrating robust reactivation of HIV-1 LTR by dCas9-SAM/msgRNAs but not SAHA in TZM-bI and CHME5 cells. FIG. 12A, FIG. 12B, FIG. 12C: The dCas9-VPH stable TZM-bI cells were treated with SAHA or infected with indicated msgRNAs lentivirus for 2-4 d before luminescence reporter assay (FIG. 12A) and CELLTITER-GLO® luminescent cell viability assay (FIG. 12B). Relative reactivation change was normalized by cell viability (FIG. 12C). FIG. 12D, FIG. 12E, FIG. 12F, FIG. 12G: The dCas9-VPH stable CHME5 cells were treated with SAHA or infected with indicated msgRNAs lentivirus for 4 d before EGFP flow cytometry for reactivation efficiency (FIG. 12D), total (FIG. 12E) and individual (FIG. 12F) fluorescent intensity and survival cell number (FIG. 12G). All data represent mean±SEM of 3-4 independent experiments. *p<0.05 and **p<0.01 indicate statistical significance by ANOVA and FLSD test as compared with corresponding vehicle control or LTR-zero control.

FIG. 13 is an overview of the dCas9-VPH SAM system using MS2-mediated sgRNA to direct multiple activators to the enhancer region of the HIV-1 promoter and reactivate viral protein expression in HIV-1 latent cells, which further induce suicide death and/or trigger host immune response to kill HIV-1 latent cells.

FIG. 14A, FIG. 14B, and FIG. 14C, depict the results of experiments demonstrating robust and persistent reactivation of HIV-1 EGFP reporter by dCas9-VPH/msgRNA in latent CHME5 microglial cells. FIG. 14A: Illustration of dCas9-VP64/MS2-p65-HSF1 (VPH) and MS2-mediated single guide RNAs (msgRNAs) in activating HIV-1-EGFP reporter virus in CHME5 cells. FIG. 14B, FIG. 14C: Cells were infected with pMSCV-LTR-dCas9-VP64-puromycin and Lenti-MS2-p65-HSF1-hygromycin (dCas9-VPH) and selected with puromycin (1 μg/ml) and hygromycin (1 μg/ml) for 10 days. The population cells were infected with indicated msgRNA-Zeocin lentivirus. The fluorescent micrographs were taken at 2 dpi (FIG. 14B). Cells were continuously cultured and selected with triple antibiotics (puromycin-hygromycin-Zeocin) for 3 days before flow cytometry with EGFP (FIG. 14C).

FIG. 15A, FIG. 15B, and FIG. 15C depict the results of experiments demonstrating persistent reactivation of HIV-1 EGFP reporter virus and suicide cell death by dCas9-VPH/msgRNA in HIV-1 latent cells. FIG. 15A: CHME5 cells were infected with indicated lentiviruses and cultured for 5 d in the absence or presence of P/H/Z triple selection (puromycin-hygromycin-Zeocin). Flow cytometry analysis was performed for total and individual EGFP fluorescent intensity. FIG. 15B, FIG. 15C: The 2D10 cells were infected for 1 and 4 d, and flow cytometry was performed for total and individual EGFP fluorescent intensity (FIG. 15B) and the EGFP reactivation efficiency (FIG. 15C).

FIG. 16A, and FIG. 16B, depict the results of experiments using EcoHIV-1eLuc. FIG. 16A: LTR-L and O induced additive reactivation of EcoHIV-eLuc reporter. The dCas9-VPH HEK293T cells were transfected with an equal amount of indicated msgRNAs LTR-L or O followed by ONE-Glo™ luciferase assay. Data represent mean±SEM of 5 independent transfections. The right column shows the additive data from L+Zero and O+Zero groups. FIG. 16B: Schematic representation of EcoHIV-1 containing enhanced firefly luciferase (eLuc). The eLuc gene was inserted after env which is driven by the 5′ LTR after the alternative splicing. For bicistronic expression, a self-cleaving 2A peptide from porcine teschovirus-1(P2A) was inserted between eLuc and nef. The envelope of HIV-1 was replaced with gp80 from murine leukemia viruses for construction of EcoHIV.

### DETAILED DESCRIPTION

The present invention relates to compositions and methods for the treatment or prevention of a HIV infection in a subject in need thereof, by reactivation of latent HIV in combination with viral clearance. For example, the present invention provides compositions and methods for the reactivation of latent HIV.

In one embodiment, the present invention uses the catalytically deficient Clustered Regularly Interspaced Short Palindromic Repeats (CRISPR)/Cas9 system (dCas9) and its fused transcriptional activators, designated as dCas9-TA system, to specifically activate HIV transcription through gRNAs that target a region of interest of the HIV genome. In one embodiment, the present invention uses the dCas9-TA system in combination with the MS2 bacteriophage coat protein-mediated synergistic activation mediator (SAM) system to provide enhanced transcriptional activation of the HIV genome through MS2-binding gRNAs. In certain embodiments, once reactivated, HIV may be cleared by the host immune system or using any viral clearance methodology known in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

As used herein, the terms “nucleic acid sequence”, “polynucleotide,” and “gene” are used interchangeably throughout the specification and include complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139(1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000)); 2′-O, 3′-C-linked [3.2.0] bicycloarabinonucleosides (see e.g. N. K Christiensen, et al., J. Am. Chem. Soc., 120: 5458-5463 (1998). Such analogs include synthetic nucleosides designed to enhance binding properties, e.g., duplex or triplex stability, specificity, or the like.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Thus, for example, the terms oligopeptide, protein, and enzyme are included within the definition of polypeptide or peptide, whether produced using recombinant techniques, chemical or enzymatic synthesis, or be naturally occurring. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins. This term also includes polypeptides that have been modified or derivatized, such as by glycosylation, acetylation, phosphorylation, and the like among others. The polypeptides include natural peptides, recombinant peptides, synthetic peptides, or a combination thereof.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for the treatment or prevention of a HIV infection in a subject in need thereof, by reactivation of latent HIV in combination with viral clearance. For example, the present invention provides compositions and methods for the reactivation of latent HIV. Reactivation of latent HIV, by way of the present invention, allows for the clearance of infected cells by antiviral therapy and/or the host immune system.

In certain embodiments, the present invention provides a composition that specifically activates the transcription of the HIV genome. In one embodiment, the composition comprises a guide RNA where the guide RNA is substantially complementary to a target region of the HIV genome. In certain embodiments, the guide RNA is substantially complementary to a target region of the HIV LTR. In certain embodiments, the guide RNA is substantially complementary to a target region of the enhancer and/or core promoter region of HIV LTR.

In certain embodiments, acomposition for reactivation of a retrovirus in vitro or in vivo comprises an isolated nucleic acid encoding a guide nucleic acid, wherein the guide nucleic acid comprises a targeting nucleotide sequence directed to one or more target sequences in the retroviral genome; an isolated nucleic acid encoding a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

In certain embodiments, the CRISPR/Cas fusion protein comprises catalytically deficient Cas protein (dCas), orthologs, homologs, mutants variants or fragments thereof, fused with one or more transcriptional activators. In some embodiments, the one or more transcriptional activators comprise VP64, p65, HSF1, p65AD, Rta, Sp1, Vax, GATA4, fragments, mutants, or any combinations thereof.

In some embodiments, the RNA binding protein, fragments, mutants, derivatives or variants thereof, comprises a bacteriophage coat protein.

In certain embodiments, the composition comprises a catalytically deficient Cas9 (dCas9) fusion protein comprising dCas9 and one or more transcription activators. In one embodiment, the composition comprises a MS2 fusion protein comprising a MS2 bacteriophage coat protein and one or more transcriptional activators. The MS2 bacteriophage coat protein is referred to herein as “MS2.” In one embodiment, the guide RNA comprises a hairpin aptamer which binds to dimerized MS2. A guide RNA capable of binding MS2 protein is referred to herein as MS2-binding gRNA or MS2-gRNA. In certain embodiments, the components of the present composition form a complex comprising the guide RNA (specifically targeted to a target region of the HIV genome), the dCas9 fusion protein, and the MS2 fusion protein.

The present invention is based, in part, on the discovery that CRISPR/Cas9 technology and SAM technology can be used together to specifically target multiple sites within the HIV-LTR promoter to reactivate latent provirus in HIV-infected host cells. For example, it is demonstrated herein that guide RNA, targeted to the HIV LTR and comprising a MS2 binding aptamer, can initiate the formation of a synergistic activation mediator (SAM) activator complex comprising dCas9, MS2, and one or more transcription activators, which induces robust reactivation in HIV in latently infected cells. The one or more transcription activators can be fused with either dCas9 or MS2.

The composition also encompasses isolated nucleic acids encoding one or more of the guide RNA, dCas9 fusion protein, and MS2 fusion protein, described elsewhere herein. For example, in one embodiment, the composition comprises one or more vectors encoding one or more of the guide RNA, dCas9 fusion protein, and MS2 fusion protein.

In one embodiment, the present invention provides a method for the treatment or prevention of HIV infection in a subject in need thereof. For example, the present method allows for reactivation of latent HIV which can thus be cleared by antiviral therapy, and the reactivated HIV-infected cells can be eliminated by virus-induced cytotoxicity and/or the host immune system. In one embodiment, the method comprises administering to the subject an effective amount of a composition comprising at least one of a guide RNA, a dCas9 fusion protein, and a MS2 fusion protein, as described elsewhere herein. In certain instances the method comprises administering a composition comprising an isolated nucleic acid encoding at least one of a guide RNA, a dCas9 fusion protein, and a MS2 fusion protein.

### Composition

The present invention provides a composition for the activation of HIV in a cell latently infected with HIV. In certain aspects the composition comprises one or more of a guide nucleic acid molecule, dCas9 fusion protein, RNA binding fusion proteins, MS2 fusion protein, and/or one or more nucleic acid molecules encoding the same.

Guide Nucleic Acid Molecule

In one embodiment, the composition comprises at least one isolated guide nucleic acid molecule, or fragment thereof, where the guide nucleic acid molecule comprises a targeting nucleotide sequence that is directed to a target site of the HIV genome. In one embodiment the guide nucleic acid is a guide RNA (gRNA).

In one embodiment, the gRNA comprises a CRISPR RNA (cRNA):trans activating cRNA (tracrRNA) duplex. In one embodiment, the gRNA comprises a stem-loop that mimics the natural duplex between the crRNA and tracrRNA. In one embodiment, the stem-loop comprises a nucleotide sequence comprising AGAAAU. For example in one embodiment, the composition comprises a synthetic or chimeric guide RNA comprising a crRNA, stem, and tracrRNA.

In certain embodiments, the composition comprises an isolated crRNA and/or an isolated tracrRNA which hybridize to form a natural duplex. For example, in one embodiment, the gRNA comprises a crRNA or crRNA precursor (pre-crRNA) comprising a targeting sequence.

In one embodiment, the gRNA comprises a targeting nucleotide sequence that is directed to a target site in the HIV genome. For example, the HIV-1 genome may comprise one or more target sequences present in the sense or antisense strand. The target sequence in the HIV genome may be any sequence in any coding or non-coding region where gRNA-mediated localization of one or more transcription activators, would result in increased activation/reactivation of the transcription of the HIV genome, or portion thereof. In certain embodiments the target sequence is about 10-30 nucleotides in length. For example, exemplary target sequences are provided in Table 1 and Table 2.

In one embodiment, the target sequence is present in HIV-LTR. In one embodiment, the target sequence is present in the enhancer and/or core promoter region of HIV-LTR.

In one embodiment, the targeting nucleotide sequence of the gRNA is designed to bind or hybridize to the target sequence of the HIV genome, and thus the targeting nucleotide sequence of the gRNA is substantially complementary to the target sequence of the HIV-1 genome. In one embodiment, the targeting nucleotide sequence of the gRNA is designed to bind or hybridize to the nucleic acid sequence of the opposite strand which is complementary to the target sequence, and thus the targeting nucleotide sequence of the gRNA is substantially the same as the target sequence.

For example, in one embodiment, if the target sequence of the HIV genome is present in the sense strand, the targeting nucleotide sequence of the gRNA can bind to the target sequence of the sense strand. In another embodiment, the targeting nucleotide sequence of the gRNA can bind to the antisense strand at the region complementary to the target sequence of the sense strand.

Further, the invention encompasses an isolated nucleic acid (e.g., gRNA) having substantial homology to a nucleic acid disclosed herein. In certain embodiments, the isolated nucleic acid has at least 75%, 80%, 85%, 90%, 95%, 96%, 97%, 98%, or 99% sequence homology with a nucleotide sequence of a gRNA described elsewhere herein.

The guide RNA sequence can be a sense or anti-sense sequence. In the CRISPR-Cas system derived from S. pyogenes (spCas9), the target DNA typically immediately precedes a 5′-NGG or NAG proto-spacer adjacent motif (PAM). Other Cas9 orthologs may have different PAM specificities. For example, Cas9 from S. thermophilus (stCas9) requires 5′-NNAGAA for CRISPR 1 and 5′-NGGNG for CRISPR3 and Neiseria menigiditis (nmCas9) requires 5′-NNNNGATT. Cas9 from Staphylococcus aureus subsp. aureus (saCas9) requires 5′-NNGRRT (R=A or G). The specific sequence of the guide RNA may vary, but, regardless of the sequence, useful guide RNA sequences will be those that minimize off-target effects while achieving high efficiency activation of HIV transcription.

In certain embodiments, the composition comprises multiple different gRNA molecules, each targeted to a different target sequence. In certain embodiments, this multiplexed strategy provides for increased efficacy. These multiplex gRNAs can be expressed separately in different vectors or expressed in one single vector.

In certain embodiments, the gRNA comprises a hairpin aptamer capable of binding MS2 protein. In certain instances, the hairpin aptamer of the gRNA binds to a MS2 fusion protein, as described elsewhere herein, thereby allowing formation of a SAM complex, see for example Konermann et al., 2015, Nature, 517(7536): 583-588, the contents of which are herein incorporated by reference.

In certain embodiments, the RNA molecules (e.g., crRNA, tracrRNA, gRNA) may be engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N⁶, N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine. The methods of the present invention or others in the art can be used to identify additional modified RNA molecules.

The isolated nucleic acid molecules of the invention, including the RNA molecules (e.g., crRNA, tracrRNA, gRNA) or nucleic acids encoding the RNA molecules, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid molecule (e.g., using automated DNA synthesis in the 3′ to 5′ direction using phosphoramidite technology) or as a series of oligonucleotides. Isolated nucleic acids of the invention also can be obtained by mutagenesis of, e.g., a naturally occurring portion crRNA, tracrRNA, RNA-encoding DNA, or of a Cas9-encoding DNA

In certain embodiments, the isolated RNA molecules are synthesized from an expression vector encoding the RNA molecule, as described in detail elsewhere herein.

Cas Protein

In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. In other embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In one embodiment, the composition comprises a CRISPR-associated (Cas) protein, or functional fragment or derivative thereof. In certain embodiments, the Cas protein is an endonuclease, including but not limited to the Cas9 nuclease. In one embodiment, the Cas protein comprises catalytically deficient Cas9 (dCas9).

In one embodiment, the Cas9 protein comprises an amino acid sequence identical to the wild type Streptococcus pyogenes Cas9 amino acid sequence. In some embodiments, the Cas protein may comprise the amino acid sequence of a Cas protein from other species, for example other Streptococcus species, such as thermophilus; Pseudomonas aeruginosa, Escherichia coli, or other sequenced bacteria genomes and archaea, or other prokaryotic microorganisms. Other Cas proteins, useful for the present invention, known or can be identified, using methods known in the art (see e.g., Esvelt et al., 2013, Nature Methods, 10: 1116-1121). In certain embodiments, the Cas protein may comprise a modified amino acid sequence, as compared to its natural source. For example, in one embodiment, the wild type Streptococcus pyogenes Cas9 sequence can be modified. For example, in certain embodiments, the Cas9 protein comprises dCas9 having point mutations D10A and H840A, thereby rendering the protein as catalytically deficient. In certain embodiments, the amino acid sequence can be codon optimized for efficient expression in human cells (i.e., “humanized) or in a species of interest.

In certain embodiments, the Cas9 protein comprises a functionally active fragment of Cas9 or dCas9. For example, in certain embodiments, the fragment of dCas9 includes a fragment which retains the functionality of bringing the gRNA to the target site. In certain instances, the functional small fragments of dCas9 make it easier for gene therapy in the clinical trials.

In one embodiment, the composition comprises a dCas9 fusion protein comprising the dCas9 protein fused to one or more transcriptional activators. For example, in one embodiment, the dCas9 fusion protein comprises the transcription activator VP64. The amino acid sequence for an exemplary dCas9 fusion protein comprising VP64 is provided in the Examples section.

However, the composition is not limited to any particular transcriptional activators and utilizes universal transcriptional activators. Thus, it can activate any target genes, including various virus, host cellular genes, mammalian cellular genes or any other species from which a transcriptional activator is selected. The specificity of the composition relies on the guide RNA that targets a specific sequence. For example, the dCas9 fusion protein may comprise any transcriptional activator known in the art that, once localized to the target sequence, can induce the activation/reactivation of a latent retrovirus, e.g. HIV. Exemplary transcriptional activators include, but are not limited to, p65AD, HSF1, Rta, Sp1, Vax, GATA4, and the like.

The invention should also be construed to include any form of a protein having substantial homology to a Cas protein (e.g., Cas9, dCas9, dCas9 fusion protein) disclosed herein. Preferably, a protein which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a Cas protein disclosed herein.

RNA Binding Proteins

In an embodiment, a composition for reactivation of a retrovirus in vitro or in vivo comprises an RNA binding protein, fragments, mutants, derivatives or variants thereof, fused to one or more transcriptional activators. The RNA binding protein can be any type of RNA binding protein from any species (see, for example, Cook K. B. et al., Nucleic Acids Res. 2011, January; v. 39 (Database issue):D301-D308; RNA-Binding protein database (RBPDB) University of Toronto (rbpdb.ccbr.utoronto.ca)). In one embodiment, the RNA binding fusion protein comprises the RNA binding protein, fragments, mutants, derivatives or variants thereof, fused to one or more transcriptional activators. In some embodiments, the RNA binding protein, fragments, mutants, derivatives or variants, comprises a bacteriophage coat protein. In another embodiment, the bacteriophage coat protein fused to one or more transcriptional activators.

In one embodiment, the composition comprises a MS2 fusion protein. In certain embodiments, the MS2 fusion protein comprises the MS2 bacteriophage coat protein fused to one or more transcription activators. The MS2 of the MS2 fusion protein is capable of binding to the gRNA thereby forming a SAM complex comprising the gRNA, dCas9 fusion protein, and MS2 fusion protein.

In one embodiment, the composition comprises a MS2 fusion protein comprising the MS2 protein fused to one or more transcription activators. For example, in one embodiment, the MS2 fusion protein comprises the transcription activators p65 and HSF1. The amino acid sequence for an exemplary MS2 fusion protein comprising p65 and HSF1 is provided in the Examples section.

However, the composition is not limited to any particular transcription activator. That is, the MS2 fusion protein may comprise any transcription activator known in the art that, once localized to the target sequence, can induce the activation/reactivation of latent HIV. Exemplary transcription activators include, but are not limited to, p65AD, HSF1, Rta, Sp1, Vax, GATA4, and the like.

The invention should also be construed to include any form of a protein having substantial homology to a MS2 protein (e.g., MS2 fusion protein) disclosed herein. Preferably, a protein which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a MS2 protein disclosed herein.

The protein may alternatively be made by recombinant means or by cleavage from a longer polyprotein. The composition of a protein may be confirmed by amino acid analysis or sequencing.

The variants of the proteins according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the protein is an alternative splice variant of the protein of the present invention, (iv) fragments of the proteins and/or (v) one in which the protein is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two proteins is determined by comparing the amino acid sequence and its conserved amino acid substitutes of one polypeptide to a sequence of a second polypeptide. Variants are defined to include protein sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two proteins is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The proteins of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The proteins of the invention may include unnatural amino acids formed by post-translational modification or by introducing unnatural amino acids during translation. A variety of approaches are available for introducing unnatural amino acids during protein translation.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the Cas protein.

A peptide or protein of the invention may be phosphorylated using conventional methods such as the method described in Reedijk et al. (The EMBO Journal 11(4):1365, 1992).

Cyclic derivatives of the proteins of the invention are also part of the present invention. Cyclization may allow the protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic proteins may comprise a beta-turn in the right position. Beta-turns may be introduced into the proteins of the invention by adding the amino acids Pro-Gly at the right position.

It may be desirable to produce a cyclic protein which is more flexible than the cyclic proteins containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The protein is more flexible as a result of the length of the disulfide linkage and the smaller number of hydrogen bonds in the beta-sheet portion. The relative flexibility of a cyclic protein can be determined by molecular dynamics simulations.

The invention also relates to proteins comprising a Cas protein fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue (e.g. cancerous tissue). A targeting domain may target the protein of the invention to a cellular component. In certain embodiments, the targeting domain targets a tumor-specific antigen or tumor-associated antigen.

N-terminal or C-terminal fusion proteins comprising a protein or chimeric protein of the invention conjugated with other molecules may be prepared by fusing, through recombinant techniques, the N-terminal or C-terminal of the protein or chimeric protein, and the sequence of a selected protein or selectable marker with a desired biological function. The resultant fusion proteins contain the Cas protein or chimeric protein fused to the selected protein or marker protein as described herein. Examples of proteins which may be used to prepare fusion proteins include immunoglobulins, glutathione-S-transferase (GST), hemagglutinin (HA), and truncated myc.

A protein of the invention may be synthesized by conventional techniques. For example, the proteins of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.).

A protein of the invention may be prepared by standard chemical or biological means of protein synthesis. Biological methods include, without limitation, expression of a nucleic acid encoding a protein in a host cell or in an in vitro translation system.

Biological preparation of a protein of the invention involves expression of a nucleic acid encoding a desired protein. An expression cassette comprising such a coding sequence may be used to produce a desired protein. For example, subclones of a nucleic acid sequence encoding a protein of the invention can be produced using conventional molecular genetic manipulation for subcloning gene fragments, such as described by Sambrook et al., Molecular Cloning: A Laboratory Manual, Cold Springs Laboratory, Cold Springs Harbor, N.Y. (2012), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity.

In the context of an expression vector, the vector can be readily introduced into a host cell, e.g., mammalian, bacterial, yeast or insect cell by any method in the art. Coding sequences for a desired protein of the invention may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The expression vector can be transferred into a host cell by physical, biological or chemical means, discussed in detail elsewhere herein.

To ensure that the protein obtained from either chemical or biological synthetic techniques is the desired protein, analysis of the protein composition can be conducted. Such amino acid composition analysis may be conducted using high resolution mass spectrometry to determine the molecular weight of the protein. Alternatively, or additionally, the amino acid content of the protein can be confirmed by hydrolyzing the protein in aqueous acid, and separating, identifying and quantifying the components of the mixture using HPLC, or an amino acid analyzer. Protein sequenators, which sequentially degrade the protein and identify the amino acids in order, may also be used to determine definitely the sequence of the protein.

The proteins and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Nucleic Acids and Vectors

In one embodiment, the composition of the invention comprises an isolated nucleic acid encoding one or more elements of the CRISPR-Cas or SAM system described herein. For example, in one embodiment, the composition comprises an isolated nucleic acid encoding at least one guide nucleic acid molecule (e.g., gRNA). In one embodiment, the composition comprises an isolated nucleic acid encoding a dCas9 fusion protein, or functional fragment or derivative thereof. In one embodiment, the composition comprises an isolated nucleic acid encoding a MS2 fusion protein, or functional fragment or derivative thereof.

In one embodiment, the composition comprises an isolated nucleic acid encoding at least one guide nucleic acid molecule (e.g., gRNA) and encoding a dCas9 fusion protein, or functional fragment or derivative thereof.

In one embodiment, the composition comprises an isolated nucleic acid encoding at least one gRNA and encoding a MS2 fusion protein.

In one embodiment, the composition comprises an isolated nucleic acid encoding a dCas9 fusion protein and encoding a MS2 fusion protein.

In one embodiment, the composition comprises an isolated nucleic acid encoding at least one gRNA, a dCas9 fusion protein, and a MS2 fusion protein.

In one embodiment, the composition comprises an isolated nucleic acid molecule encoding at least one guide nucleic acid molecule (e.g., gRNA) and further comprises an isolated nucleic acid encoding a dCas9 fusion protein, or functional fragment or derivative thereof.

In one embodiment, the composition comprises an isolated nucleic acid molecule encoding at least one guide nucleic acid molecule (e.g., gRNA) and further comprises an isolated nucleic acid encoding a MS2 fusion protein, or functional fragment or derivative thereof.

In one embodiment, the composition comprises an isolated nucleic acid molecule encoding at least one guide nucleic acid molecule (e.g., gRNA), an isolated nucleic acid encoding a dCas9 fusion protein or functional fragment or derivative thereof, and an isolated nucleic acid encoding a MS2 fusion protein, or functional fragment or derivative thereof.

In one embodiment, the composition comprises at least one isolated nucleic acid encoding a gRNA, where the gRNA comprises a targeting nucleotide sequence directed to a target sequence of HIV LTR described in Table 1 or Table 2. In one embodiment, the composition comprises at least one isolated nucleic acid encoding a gRNA, where the gRNA comprises a targeting nucleotide sequence having at least 75%, 80%, 85%, 90%, 95%, 96%, 97%, 98%, or 99% sequence homology with a targeting nucleotide sequence directed to a target sequence of HIV LTR described in Table 1 or Table 2.

In one embodiment, the composition comprises at least one isolated nucleic acid encoding a dCas9 fusion protein described elsewhere herein, or a functional fragment or derivative thereof. In one embodiment, the composition comprises at least one isolated nucleic acid encoding a dCas9 protein having at least 75%, 80%, 85%, 90%, 95%, 96%, 97%, 98%, or 99% amino acid sequence homology with a dCas9 protein described elsewhere herein.

In one embodiment, the composition comprises at least one isolated nucleic acid encoding a MS2 fusion protein described elsewhere herein, or a functional fragment or derivative thereof. In one embodiment, the composition comprises at least one isolated nucleic acid encoding a MS2 protein having at least 75%, 80%, 85%, 90%, 95%, 96%, 97%, 98%, or 99% amino acid sequence homology with a MS2 protein described elsewhere herein.

The isolated nucleic acid may comprise any type of nucleic acid, including, but not limited to DNA and RNA. For example, in one embodiment, the composition comprises an isolated DNA molecule, including for example, an isolated cDNA molecule, encoding a gRNA or protein of the invention, or functional fragment thereof. In one embodiment, the composition comprises an isolated RNA molecule encoding a protein of the invention, or a functional fragment thereof. The isolated nucleic acids may be synthesized using any method known in the art.

The present invention also includes a vector in which the isolated nucleic acid of the present invention is inserted. The art is replete with suitable vectors that are useful in the present invention. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

In brief summary, the expression of natural or synthetic nucleic acids encoding an RNA and/or peptide is typically achieved by operably linking a nucleic acid encoding the RNA and/or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. The Rous sarcoma virus (RSV) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of the nucleic acid and/or protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, nanoparticles, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular protein, e.g., by immunological means (ELISAs and Western blots) or by assays described herein to identify agents falling within the scope of the invention.

In certain embodiments, the composition comprises a cell genetically modified to express one or more isolated nucleic acids and/or proteins described herein. For example, the cell may be transfected or transformed with one or more vectors comprising an isolated nucleic acid sequence encoding a gRNA, dCas9 fusion protein, and/or MS2 fusion protein. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are a self-derived cell, make it possible to avoid rejection reactions, which are the biggest obstacle to regenerative medicine or transplantation therapy.

Pharmaceutical Compositions

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

The present invention also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

Administration of the compositions of this invention may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension the composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

### Methods of Treatment

The present invention provides a method of treating or preventing HIV in a subject. For example, in certain embodiments, the invention provides a method for activating HIV in a latently infected cell. Activation or reactivation of latent HIV, by way of the present invention allows for clearing of HIV by the viral-protein induced cytotoxicity and/or host immune system and by using concurrent or subsequent antiviral therapy. In one embodiment, the method comprises administering to a subject in need thereof, an effective amount of a composition comprising at least one of a guide nucleic acid molecule, a dCas9 fusion protein, a MS2 fusion protein, or functional fragments or derivatives thereof.

In one embodiment, the method comprises administering a composition comprising an isolated nucleic acid encoding at least one of: the guide nucleic acid molecule, a dCas9 fusion protein, a MS2 fusion protein, or functional fragments or derivatives thereof. In certain embodiments, the method comprises administering a composition described herein to a subject diagnosed with a HIV infection, at risk for developing a HIV infection, a subject with a latent HIV infection, and the like.

The methods of the invention are also employed for treatment or prevention of diseases and disorders associated with HIV infections.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs. The therapeutic agents may be administered under a metronomic regimen. As used herein, “metronomic” therapy refers to the administration of continuous low-doses of a therapeutic agent.

The compositions can be administered in conjunction with (e.g., before, simultaneously or following) one or more therapies. For example, in certain embodiments, the method comprises administration of a composition of the invention in conjunction with an additional antiviral or anti-HIV therapy.

Dosage, toxicity and therapeutic efficacy of the present compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The gRNA/dCas9 fusion protein/MS2 fusion protein compositions that exhibit high therapeutic indices are preferred. While gRNA/dCas9 fusion protein/MS2 fusion protein compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The gRNA expression cassette can be delivered to a subject by methods known in the art. In some aspects, the dCas9 may be a fragment wherein the active domains or domains necessary for localization of the transcription activator are included, thereby cutting down on the size of the molecule. Thus, the molecules can be used clinically, similar to the approaches taken by current gene therapy.

In one embodiment, the method comprises genetically modifying a cell to express a guide nucleic acid molecule, a dCas9 fusion protein, and a MS2 fusion protein. For example, in one embodiment, the method comprises contacting a cell with one or more isolated nucleic acids encoding the guide nucleic acid, dCas9 fusion protein, and MS2 fusion protein.

In one embodiment, the cell is genetically modified in vivo in the subject in whom the therapy is intended. In certain aspects, for in vivo, delivery the nucleic acid is injected directly into the subject. For example, in one embodiment, the nucleic acid is delivered at the site where the composition is required. In vivo nucleic acid transfer techniques include, but is not limited to, transfection with viral vectors such as adenovirus, Herpes simplex I virus, adeno-associated virus), lipid-based systems (useful lipids for lipid-mediated transfer of the gene are DOTMA, DOPE and DC-Chol, for example), naked DNA, and transposon-based expression systems. Exemplary gene therapy protocols see Anderson et al., Science 256:808-813 (1992). See also WO 93/25673 and the references cited therein. In certain embodiments, the method comprises administering of RNA, for example mRNA, directly into the subject (see for example, Zangi et al., 2013 Nature Biotechnology, 31: 898-907).

For ex vivo treatment, an isolated cell is modified in an ex vivo or in vitro environment. In one embodiment, the cell is autologous to a subject to whom the therapy is intended. Alternatively, the cell can be allogeneic, syngeneic, or xenogeneic with respect to the subject. The modified cells may then be administered to the subject directly.

One skilled in the art recognizes that different methods of delivery may be utilized to administer an isolated nucleic acid into a cell. Examples include: (1) methods utilizing physical means, such as electroporation (electricity), a gene gun (physical force) or applying large volumes of a liquid (pressure); and (2) methods wherein the nucleic acid or vector is complexed to another entity, such as a liposome, aggregated protein or transporter molecule.

The amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

Genetically modified cells may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: CRISPR/Cas9-Mediated Cell-Specific “Shock and Kill” for HIV-1 Cure

The rapidly developing genome editing technology provides a novel and personalized approach to purge the HIV latent reservoir in a target-specific manner. Such proof of concept has been tested using ZFN and TALEN techniques. Described herein is the feasibility and higher efficiency of CRISPR/Cas9 synergistic activation mediator (SAM) technology in shocking the HIV latent reservoir at the cellular level.

Experiments were conducted to identify the effective guide RNAs (gRNAs) targeting HIV-1 LTR in activating HIV-1 promoter. Further, the efficacy of dCas9-VP64/gRNA alone or dCas9-VP64/MS2-p65-HSF1/MS2-gRNA combination in inducing reactivation of HIV-1 latent reservoir was compared. Experiments were also conducted to evaluate the efficiency of HIV-1 reactivation by Cas9-SAM technology in different HIV-1 latent cells.

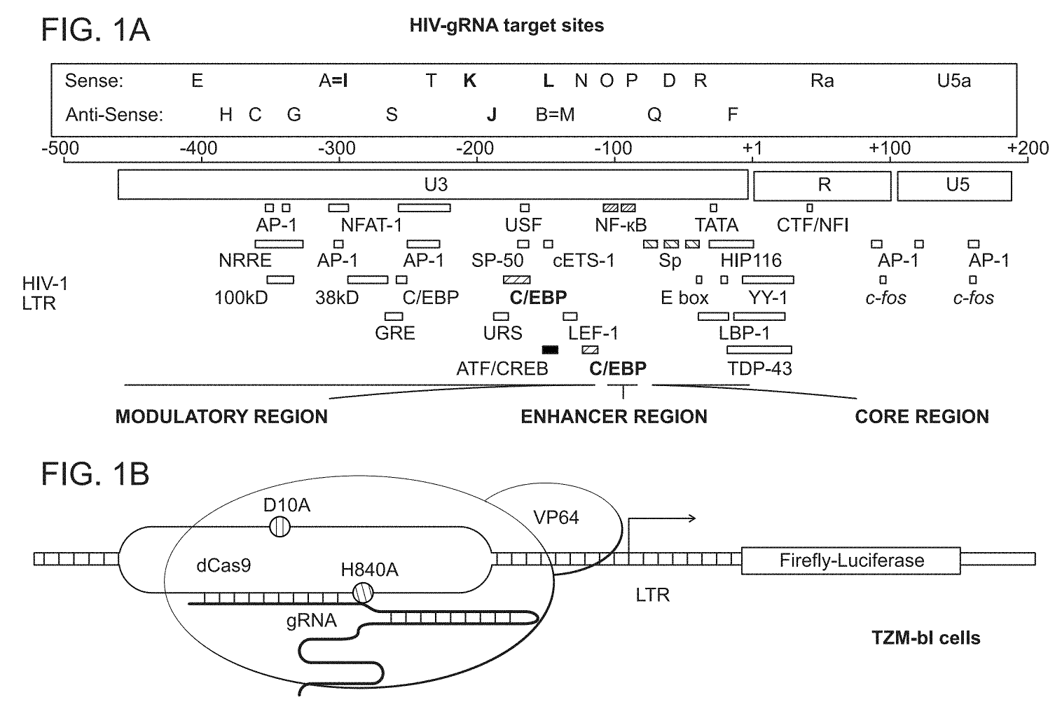

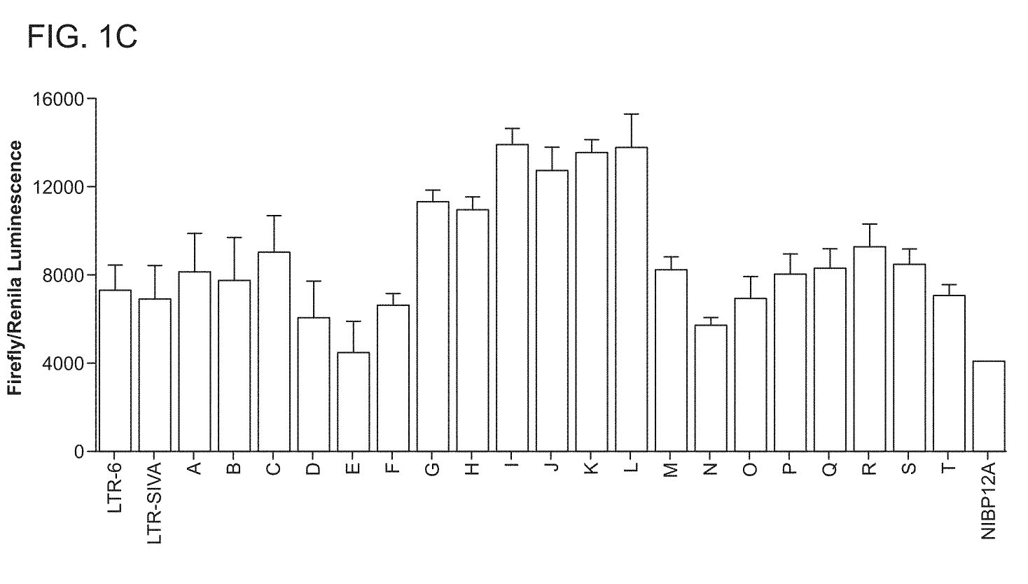

Bioinformatics was used to identify potential gRNAs targeting HIV-1 LTR promoter region (FIG. 1A). Predicted HIV-1 LTR target sequences (in both sense and antisense strands) using only NGG as the PAM, is provided in Table 1. gRNA expression cassettes were cloned into modified pKLV-gRNA(modified BbsI)-Puro-2A-BFP lentiviral vector (Addgene #50946) and Lenti-gRNA(BsmBI)-MS2-EF1a-Zeo vector (Addgene #61427). The target seed sequence (20 bp DNA, Table 2) is cloned into a sgRNA expression vector and is transcribed by U6 promoter into RNA (complement). The transcribed RNA sequence (20 bp) together with the crRNA:tracRNA (all together called single guide-RNA, sgRNA) binds to the target DNA sequence due to nucleotide complementation.

Cultured cells were transfected using lipofectamine3000 transfection or lentivirus infection. The firefly-luciferase reporter assay was used using Envision multiplate reader. Treated and controls were imaged using fluorescent microscopy and/or were analyzed using flow cytometry.

Experiments were first performed using TZM-bI cells, transfected to express various gRNA targeting HIV-1 LTR and dCas9-VP64 (FIG. 1B). It was observed that this strategy produce little to no noticeable impact on the reactivation of HIV-1 (FIG. 1C).

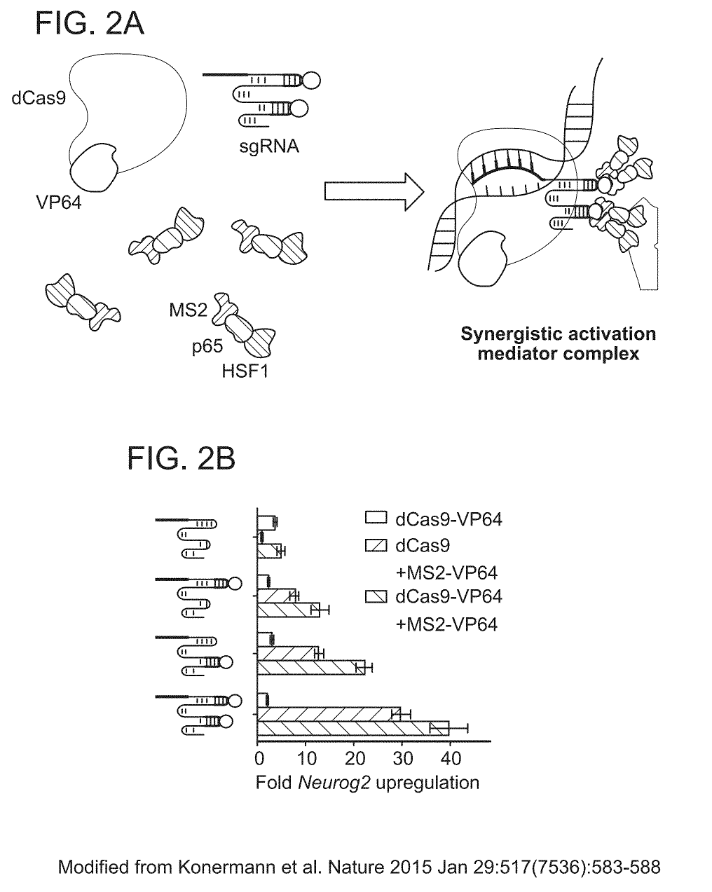

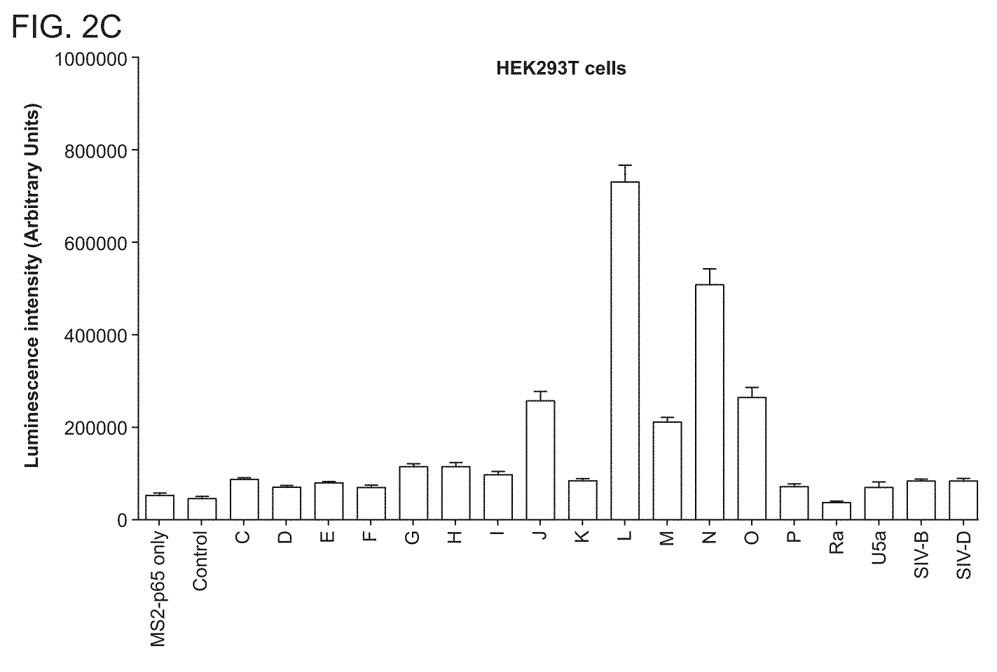

Experiments were then conducted employing a MS2-mediated synergistic activator mediator (SAM) system that includes additional p65 and HSF1 activators along with various HIV-1 LTR targeting MS2-guide RNAs (FIG. 2A and FIG. 2B) (Konermann et al., 2015, Nature, 517(7536): 583-588). Several specific guide RNAs were identified that stimulate viral gene transcription by up to 13-fold in HEK293T cells (FIG. 2C, Table 2).

It was found that the most effective gRNAs tested (J, L, M, N, O) are targeted around the enhancer region (close to NFκB, CEBPβ and GATA-1). The LTR-L covers partial of both CEBPβ and GATA-1. The LTR-O covers the first NFκB site showed 4.8 fold increase but the LTR-P (9 basepair identical to LTR-O) has only 1.32 fold increase although the LTR-P covers partial NFκB site 1 and almost all NFκB site 2. Screening of other gRNAs that target the enhancer and/or core promoter may identify more effective gRNAs (see FIG. 1A).

Table 2 provides the seed sequence for each of the guide RNAs tested, along with the fold increase observed as sgRNA during delivery of sgRNA/dCas9-VP64 and as MS2-sgRNA during delivery of MS2-sgRNA/dCas9-VP64/MS2-p65-HSF1.

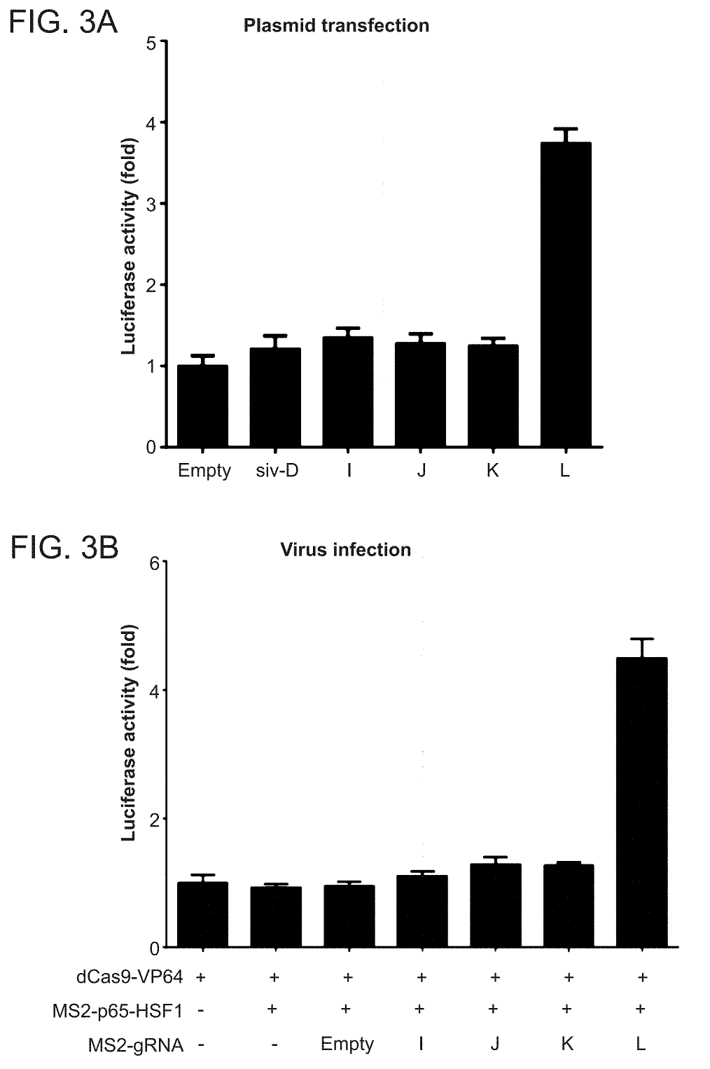

Experiments were conducted to compare reactivation of HIV-LTR luciferase reporter in TZM-BI cells induced by gRNA/dCas9-VP64/MS2-p65-HSF1/delivered by either transient plasmid transfection (FIG. 3A) or stable lentivirus infection (FIG. 3B). Cells were plated in 96-well plate and the various MS2-gRNAs were cotransfected by Lipofectamine 3000 (FIG. 3A) or coinfected by lentiviruses (FIG. 3B, MOI=50) with pMSCV-LTR-dCas9-VP64-BFP plus pLV-MS2-p65-HSF1-GFP. Two days later, OneGlo luminescence assay was performed. The SIV MS2-gRNA-D was used as negative control. It was observed that MS2-p65-HSF1 alone did not influence the basal activity of LTR-luciferase reporter. Further, the gRNA-J has no effect due to 2 nucleotides mismatch to the LTR sequence from TZM-bI cells, implying the specificity of the system. Further, it was observed that delivery by transfection or lentiviral infection induced similar reactivation of HIV-LTR.

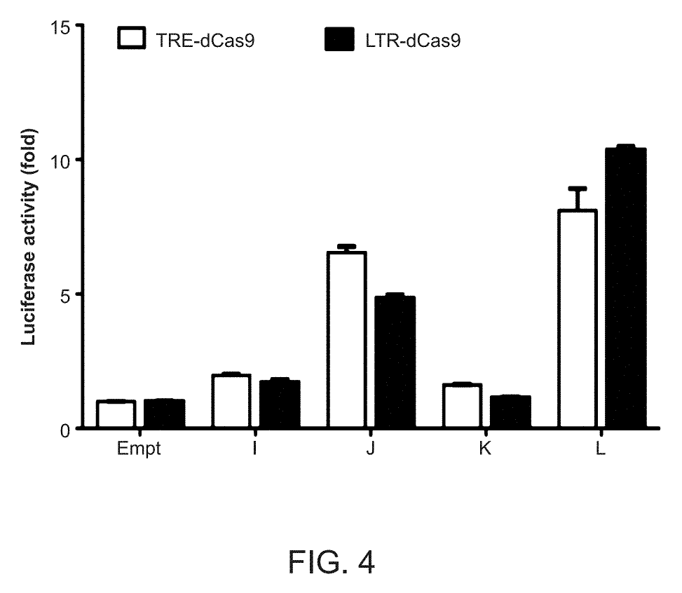

Experiments were conducted using inducible and constitutive dCas9-VP64 expression. The various MS2-gRNAs were cotransfected with Tet-inducible pHAGE-TRE-dCas9-VP64 or constitutive pMSCV-LTR-dCas9-VP64-BFP plus pLV-MS2-p65-HSF1-GFP and pNL4-3-EcoHIV-eLuc. Two days later, OneGlo luminescence assay was performed. It was observed that inducible and constitutive expression of dCas9-VP64 induced similar activation of EcoHIV luciferase reporter in HEK293T cells (FIG. 4).

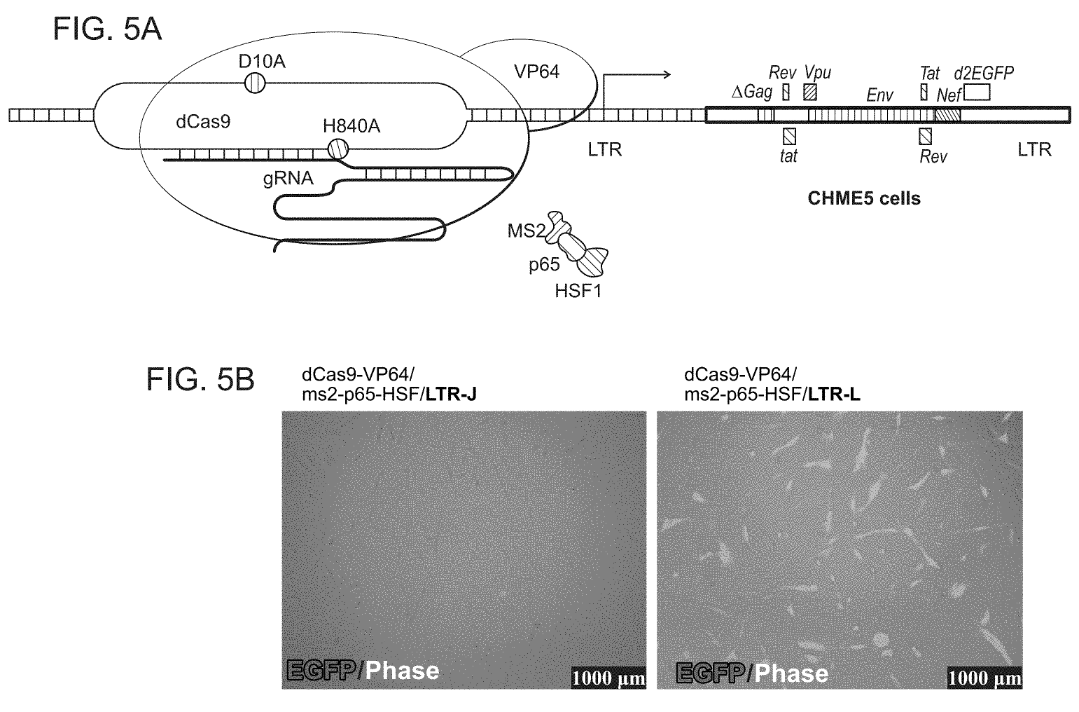

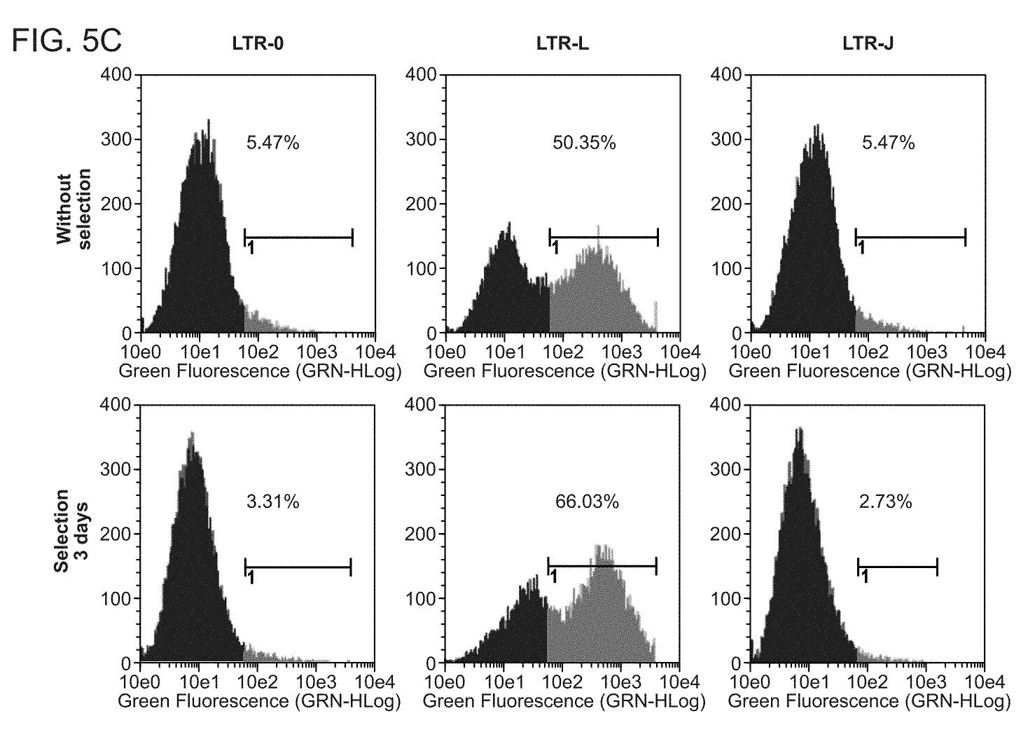

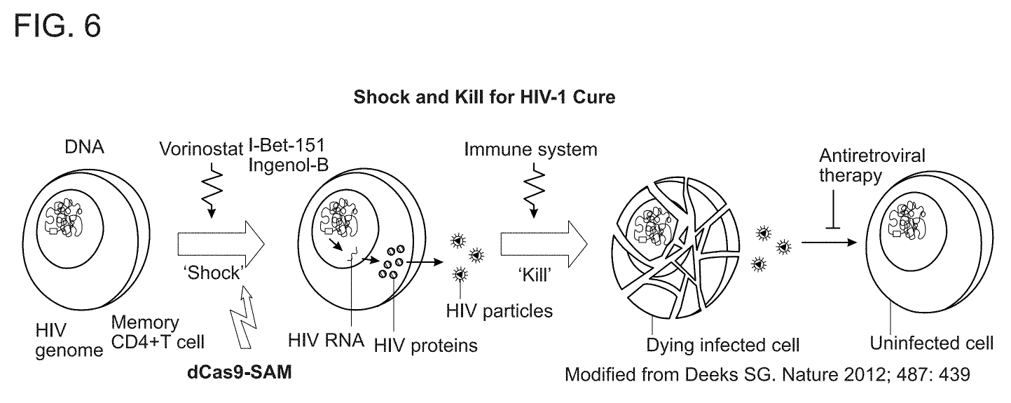

Experiments were conducted to examine reactivation of HIV-1 LTR in latent CHME5 microglial cells (FIG. 5A). Cells were infected with pMSCV-LTR-dCas9-VP64-puromycin and Lenti-MS2-p65-HSF1-hygromysin, selected for 10 days. The population cells were infected with Lenti-MS2-gRNAs-Zeosin and the fluorescent micrographs were taken at 1 day after infection. Increased EGFP, indicative of reactivation, was observed when cells were infected with gRNA LTR-L (FIG. 5B). Cells were continuously cultured for 7 days, and selected with triple antibiotics (puromycin-hygromycin-Zeocin) for 3 days before flow cytometry, where increased green fluorescence was observed in cells treated with gRNA LTR-L (FIG. 5C). These data suggest that the dCas9-SAM technology induced dramatic and persistent reactivation of HIV latent reservoir.

The data presented herein demonstrates that HIV-1 LTR-gRNAs with dCas9-VP64 alone did not induce any noticeable impact on HIV-1 reactivation, while MS2-gRNAs and MS2-p65-HSF1, in combination with dCas9-VP64 induced robust reactivation in HIV-1 latent infected cells. It was found that 7 of 16 MS2-gRNAs are most effective and target the core and enhancer promoter region of HIV-1 LTR. This latency-reversing dCas9-SAM system offers a highly specific, highly efficient and lowly toxic new biological approach for employment in the “shock and kill” strategy to eliminate HIV-1 latent reservoir in patients.

HIV-1 LTR sequence (634 bp; SEQ ID NO: 1)
TGGAAGGGCTAATTCACTCCCAACGAAGACAAGATATCCTTGATCTGT
GGATCTACCACACACAAGGCTACTTCCCTGATTGGCAGAACTACACAC
CAGGGCCAGGGATCAGATATCCACTGACCTTTGGATGGTGCTACAAGC
TAGTACCAGTTGAGCAAGAGAAGGTAGAAGAAGCCAATGAAGGAGAGA
ACACCCGCTTGTTACACCCTGTGAGCCTGCATGGGATGGATGACCCGG
AGAGAGAAGTATTAGAGTGGAGGTTTGACAGCCGCCTAGCATTTCATC
ACATGGCCCGAGAGCTGCATCCGGAGTACTTCAAGAACTGCTGACATC
GAGCTTGCTACAAGGGACTTTCCGCTGGGGACTTTCCAGGGAGGCGTG
GCCTGGGCGGGACTGGGGAGTGGCGAGCCCTCAGATGCTGCATATAAG
CAGCTGCTTTTTGCTTGTACTGGGTCTCTCTGGTTAGACCAGATCTGA
GCCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTGT
GACTCTGGTAACTAGAGATCCCTCAGACCCTTTTAGTCAGTGTGGAAA
ATCTCTAGCA

TABLE 1
HIV-1 LTR target sequence
predicted by sgRNA Designer from MIT

| Extended Spacer | |
|---|---|
| (NNNN[20nt]NGGNNN) | Strand |
| CGAAGACAAGATATCCTTGATCT | sense |
| GTGGATC (SEQ ID NO: 2) | |
| TGATCTGTGGATCTACCACACAC | sense |
| AAGGCTA (SEQ ID NO: 3) | |
| CACACACAAGGCTACTTCCCTGA | sense |
| TTGGCAG (SEQ ID NO: 4) | |
| CCCTGATTGGCAGAACTACACAC | sense |
| CAGGGCC (SEQ ID NO: 5) | |
| CCTGATTGGCAGAACTACACACC | sense |
| AGGGCCA (SEQ ID NO: 6) | |
| TTGGCAGAACTACACACCAGGGC | sense |
| CAGGGAT (SEQ ID NO: 7) | |
| TGGCAGAACTACACACCAGGGCC | sense |
| AGGGATC (SEQ ID NO: 8) | |
| AGGGATCAGATATCCACTGACCT | sense |
| TTGGATG (SEQ ID NO: 9) | |
| ATCAGATATCCACTGACCTTTGG | sense |
| ATGGTGC (SEQ ID NO: 10) | |
| AGCTAGTACCAGTTGAGCAAGAG | sense |
| AAGGTAG (SEQ ID NO: 11) | |
| AGAGAAGGTAGAAGAAGCCAATG | sense |
| AAGGAGA (SEQ ID NO: 12) | |
| CTTGTTACACCCTGTGAGCCTGC | sense |
| ATGGGAT (SEQ ID NO: 13) | |
| TTGTTACACCCTGTGAGCCTGCA | sense |
| TGGGATG (SEQ ID NO: 14) | |
| TACACCCTGTGAGCCTGCATGGG | sense |
| ATGGATG (SEQ ID NO: 15) | |
| TGAGCCTGCATGGGATGGATGAC | sense |
| CCGGAGA (SEQ ID NO: 16) | |
| GACCCGGAGAGAGAAGTATTAGA | sense |
| GTGGAGG (SEQ ID NO: 17) | |
| CCGGAGAGAGAAGTATTAGAGTG | sense |
| GAGGTTT (SEQ ID NO: 18) | |
| ACAGCCGCCTAGCATTTCATCAC | sense |
| ATGGCCC (SEQ ID NO: 19) | |
| ATCACATGGCCCGAGAGCTGCAT | sense |
| CCGGAGT (SEQ ID NO: 20) | |
| ACTGCTGACATCGAGCTTGCTAC | sense |
| AAGGGAC (SEQ ID NO: 21) | |
| CTGCTGACATCGAGCTTGCTACA | sense |
| AGGGACT (SEQ ID NO: 22) | |
| AGCTTGCTACAAGGGACTTTCCG | sense |
| CTGGGGA (SEQ ID NO: 23) | |
| GCTTGCTACAAGGGACTTTCCGC | sense |
| TGGGGAC (SEQ ID NO: 24) | |
| CTTGCTACAAGGGACTTTCCGCT | sense |
| GGGGACT (SEQ ID NO: 25) | |
| GGGACTTTCCGCTGGGGACTTTC | sense |
| CAGGGAG (SEQ ID NO: 26) | |
| GGACTTTCCGCTGGGGACTTTCC | sense |
| AGGGAGG (SEQ ID NO: 27) | |
| CTTTCCGCTGGGGACTTTCCAGG | sense |
| GAGGCGT (SEQ ID NO: 28) | |
| CGCTGGGGACTTTCCAGGGAGGC | sense |
| GTGGCCT (SEQ ID NO: 29) | |
| GGGACTTTCCAGGGAGGCGTGGC | sense |
| CTGGGCG (SEQ ID NO: 30) | |
| GGACTTTCCAGGGAGGCGTGGCC | sense |
| TGGGCGG (SEQ ID NO: 31) | |
| CTTTCCAGGGAGGCGTGGCCTGG | sense |
| GCGGGAC (SEQ ID NO: 32) | |
| TTTCCAGGGAGGCGTGGCCTGGG | sense |
| CGGGACT (SEQ ID NO: 33) | |
| AGGGAGGCGTGGCCTGGGCGGGA | sense |
| CTGGGGA (SEQ ID NO: 34) | |
| GGGAGGCGTGGCCTGGGCGGGAC | sense |
| TGGGGAG (SEQ ID NO: 35) | |
| GGAGGCGTGGCCTGGGCGGGACT | sense |
| GGGGAGT (SEQ ID NO: 36) | |
| CGTGGCCTGGGCGGGACTGGGGA | sense |
| GTGGCGA (SEQ ID NO: 37) | |
| CTTTTTGCTTGTACTGGGTCTCT | sense |
| CTGGTTA (SEQ ID NO: 38) | |
| CTCTGGTTAGACCAGATCTGAGC | sense |
| CTGGGAG (SEQ ID NO: 39) | |
| TCTGGTTAGACCAGATCTGAGCC | sense |
| TGGGAGC (SEQ ID NO: 40) | |
| CCAGATCTGAGCCTGGGAGCTCT | sense |
| CTGGCTA (SEQ ID NO: 41) | |
| AGCCTGGGAGCTCTCTGGCTAAC | sense |
| TAGGGAA (SEQ ID NO: 42) | |
| GCCTGGGAGCTCTCTGGCTAACT | sense |
| AGGGAAC (SEQ ID NO: 43) | |
| GTGTGCCCGTCTGTTGTGTGACT | sense |
| CTGGTAA (SEQ ID NO: 44) | |
| GATCAAGGATATCTTGTCTTCGT | antisense |
| TGGGAGT (SEQ ID NO: 45) | |
| AGATCAAGGATATCTTGTCTTCG | antisense |
| TTGGGAG (SEQ ID NO: 46) | |
| TGTGTGTGGTAGATCCACAGATC | antisense |
| AAGGATA (SEQ ID NO: 47) | |
| CAATCAGGGAAGTAGCCTTGTGT | antisense |
| GTGGTAG (SEQ ID NO: 48) | |
| CCTGGTGTGTAGTTCTGCCAATC | antisense |
| AGGGAAG (SEQ ID NO: 49) | |
| CCCTGGTGTGTAGTTCTGCCAAT | antisense |
| CAGGGAA (SEQ ID NO: 50) | |
| CAGTGGATATCTGATCCCTGGCC | antisense |
| CTGGTGT (SEQ ID NO: 51) | |
| AAAGGTCAGTGGATATCTGATCC | antisense |
| CTGGCCC (SEQ ID NO: 52) | |
| CTTGTAGCACCATCCAAAGGTCA | antisense |
| GTGGATA (SEQ ID NO: 53) | |
| GTACTAGCTTGTAGCACCATCCA | antisense |
| AAGGTCA (SEQ ID NO: 54) | |
| TTCTTCTACCTTCTCTTGCTCAA | antisense |
| CTGGTAC (SEQ ID NO: 55) | |
| ACAAGCGGGTGTTCTCTCCTTCA | antisense |
| TTGGCTT (SEQ ID NO: 56) | |
| GCAGGCTCACAGGGTGTAACAAG | antisense |
| CGGGTGT (SEQ ID NO: 57) | |
| TGCAGGCTCACAGGGTGTAACAA | antisense |
| GCGGGTG (SEQ ID NO: 58) | |
| TCATCCATCCCATGCAGGCTCAC | antisense |
| AGGGTGT (SEQ ID NO: 59) | |
| GTCATCCATCCCATGCAGGCTCA | antisense |
| CAGGGTG (SEQ ID NO: 60) | |
| CTCTCCGGGTCATCCATCCCATG | antisense |
| CAGGCTC (SEQ ID NO: 61) | |
| CTCCACTCTAATACTTCTCTCTC | antisense |
| CGGGTCA (SEQ ID NO: 62) | |
| CCTCCACTCTAATACTTCTCTCT | antisense |
| CCGGGTC (SEQ ID NO: 63) | |
| CGGGCCATGTGATGAAATGCTAG | antisense |
| GCGGCTG (SEQ ID NO: 64) | |
| TCTCGGGCCATGTGATGAAATGC | antisense |
| TAGGCGG (SEQ ID NO: 65) | |
| TGAAGTACTCCGGATGCAGCTCT | antisense |
| CGGGCCA (SEQ ID NO: 66) | |
| TTGAAGTACTCCGGATGCAGCTC | antisense |
| TCGGGCC (SEQ ID NO: 67) | |
| ATGTCAGCAGTTCTTGAAGTACT | antisense |
| CCGGATG (SEQ ID NO: 68) | |
| CACGCCTCCCTGGAAAGTCCCCA | antisense |
| GCGGAAA (SEQ ID NO: 69) | |
| AGTCCCGCCCAGGCCACGCCTCC | antisense |
| CTGGAAA (SEQ ID NO: 70) | |
| GCTCGCCACTCCCCAGTCCCGCC | antisense |
| CAGGCCA (SEQ ID NO: 71) | |
| CAGCTGCTTATATGCAGCATCTG | antisense |
| AGGGCTC (SEQ ID NO: 72) | |
| GCAGCTGCTTATATGCAGCATCT | antisense |
| GAGGGCT (SEQ ID NO: 73) | |
| CCAGAGAGCTCCCAGGCTCAGAT | antisense |
| CTGGTCT (SEQ ID NO: 74) | |
| TCCCTAGTTAGCCAGAGAGCTCC | antisense |
| CAGGCTC (SEQ ID NO: 75) | |
| AAGCTTTATTGAGGCTTAAGCAG | antisense |
| TGGGTTC (SEQ ID NO: 76) | |
| CAAGCTTTATTGAGGCTTAAGCA | antisense |
| GTGGGTT (SEQ ID NO: 77) | |
| AAGCACTCAAGGCAAGCTTTATT | antisense |
| GAGGCTT (SEQ ID NO: 78) | |
| GGCACACACTACTTGAAGCACTC | antisense |
| AAGGCAA (SEQ ID NO: 79) | |
| AGTTACCAGAGTCACACAACAGA | antisense |
| CGGGCAC (SEQ ID NO: 80) | |
| TAGTTACCAGAGTCACACAACAG | antisense |
| ACGGGCA (SEQ ID NO: 81) | |
| TCCACACTGACTAAAAGGGTCTG | antisense |
| AGGGATC (SEQ ID NO: 82) | |
| TTCCACACTGACTAAAAGGGTCT | antisense |
| GAGGGAT (SEQ ID NO: 83) | |

TABLE 2
The sequences for tested sgRNA and MS2-sgRNA

| | | | MS2- |
|---|---|---|---|
| | | sgRNA | sgRNA |
| | | TZM-bI | HEK293T |
| HIV-LTR | Seed sequence | Fold | Fold |
| MS2-p65- | | | 1.00 |
| HSF1 only | | | |
| Control | GGGTCTTCGAGAAGACCT (SEQ ID NO: 84) | 1.00 | 0.86 |
| A | GATCAGATATCCACTGACCTT (SEQ ID NO: 85) | 1.17 | |
| B | GCAGCAGTTCTTGAAGTACTC (SEQ ID NO: 86) | 1.12 | |
| C | GATTGGCAGAACTACACACC (SEQ ID NO: 87) | 1.30 | 1.58 |
| D | GCGTGGCCTGGGCGGGACTG (SEQ ID NO: 88) | 0.87 | 1.30 |
| E | GATCTGTGGATCTACCACACACA (SEQ ID NO: 89) | 0.65 | 1.45 |
| F | GCTGCTTATATGCAGCATCTGAG (SEQ ID NO: 90) | 0.96 | 1.29 |
| G | GTGTGGTAGATCCACAGATCA (SEQ ID NO: 91) | 1.63 | 2.10 |
| H | GCAGGGAAGTAGCCTTGTGTG (SEQ ID NO: 92) | 1.58 | 2.09 |
| I | GATCAGATATCCACTGACCTT (SEQ ID NO: 93) | 2.00 | 1.80 |
| J | GCACACTAATACTTCTCCCTC (SEQ ID NO: 94) | 1.83 | 4.68 |
| K | GCCTCCTAGCATTTCGTCACA (SEQ ID NO: 95) | 1.95 | 1.53 |
| L | GCATGGCCCGAGAGCTGCATC (SEQ ID NO: 96) | 1.98 | 13.29 |
| M | GCAGCAGTCTTTGTAGTACTC (SEQ ID NO: 97) | 1.18 | 3.83 |
| N | GCTGACATCGAGCTTTCTACA (SEQ ID NO: 98) | 0.83 | 9.25 |
| O | GTCTACAAGGGACTTTCCGCT (SEQ ID NO: 99) | 1.00 | 4.81 |
| P | GCTTTCCGCTGGGGACTTTCC (SEQ ID NO: 100) | 1.15 | 1.32 |
| Q | GCCTCCCTGGAAAGTCCCCAG (SEQ ID NO: 101) | 1.19 | |
| R | GCCTGGGCGGGACTGGGGAGG (SEQ ID NO: 102) | 1.33 | |
| S | GTCCATCCCATGCAGGCTCAC (SEQ ID NO: 103) | 1.22 | |
| T | GCGGAGAGAGAAGTATTAGAG (SEQ ID NO: 104) | 1.02 | |
| Ra | GCTTTATTGAGGCTTAAGCAG (SEQ ID NO: 105) | | 0.67 |
| U5a | GCCCGTCTGTTGTGTGACTC (SEQ ID NO: 106) | | 1.28 |

dCas9-VP64 amino acid sequence (SEQ ID NO: 107):
MDKKYSIGLAIGTNSVGWAVITDEYKVPSKKFKVLGNTDRHSIKKNLI
GALLFDSGETAEATRLKRTARRRYTRRKNRICYLQEIFSNEMAKVDDS
FFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTY
NQLFEENPINASGVDAKAILSARLSKSRRLENLIAQLPGEKKNGLFGN
LIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLK
ALVRQQLPEKYKEIFFDQSKNGYAGYIDGGASQEEFYKFIKPILEKMD
GTEELLVKLNREDLLRKQRTFDNGSIPHQIHLGELHAILRRQEDFYPF
LKDNREKIEKILTFRIPYYVGPLARGNSRFAWMTRKSEETITPWNFEE
VVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLT
LFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSL
HEHIANLAGSPAIKKGILQTVKVVDELVKVMGRHKPENIVIEMARENQ
TTQKGQKNSRERMKRIEEGIKELGSQILKEHPVENTQLQNEKLYLYYL
QNGRDMYVDQELDINRLSDYDVDAIVPQSFLKDDSIDNKVLTRSDKNR
GKSDNVPSEEVVKKMKNYWRQLLNAKLITQRKFDNLTKAERGGLSELD
KAGFIKRQLVETRQITKHVAQILDSRMNTKYDENDKLIREVKVITLKS
KLVSDFRKDFQFYKVREINNYHHAHDAYLNAVVGTALIKKYPKLESEF
VYGDYKVYDVRKMIAKSEQEIGKATAKYFFYSNIMNFFKTEITLANGE
IRKRPLIETNGETGEIVWDKGRDFATVRKVLSMPQVNIVKKTEVQTGG
FSKESILPKRNSDKLIARKKDWDPKKYGGFDSPTVAYSVLVVAKVEKG
KSKKLKSVKELLGITIMERSSFEKNPIDFLEAKGYKEVKKDLIIKLPK
YSLFELENGRKRMLASAGELQKGNELALPSKYVNFLYLASHYEKLKGS
PEDNEQKQLFVEQHKHYLDEIIEQISEFSKRVILADANLDKVLSAYNK
HRDKPIREQAENIIHLFTLTNLGAPAAFKYFDTTIDRKRYTSTKEVLD
ATLIHQSITGLYETRIDLSQLGGDEGAPKKKRKVGSSGSPKKKRKVGS
DALDDFDLDMLGSDALDDFDLDMLGSDALDDFDLDMLGSDALDDFDLD
ML
(Regular Font for dCas9; underlined for nuclear
localization signal; bold for 4 tandem copies of
Herpes Simplex Viral Protein VP16)
MS2-p65-HSF1 amino acid sequence
(SEQ ID NO: 108):
MASNFTQFVLVDNGGTGDVTVAPSNFANGVAEWISSNSRSQAYKVTCS
VRQSSAQKRKYTIKVEVPKVATQTVGGVELPVAAWRSYLNMELTIPIF
ATNSDCELIVKAMQGLLKDGNPIPSAIAANSGIYSAGGGGSGGGGSGG
GGSGPKKKRKVAAAGSPSGQISNQALALAPSSAPVLAQTMVPSSAMVP
LAQPPAPAPVLTPGPPQSLSAPVPKSTQAGEGTLSEALLHLQFDADED
LGALLGNSTDPGVFTDLASVDNSEFQQLLNQGVSMSHSTAEPMLMEYP
EAITRLVTGSQRPPDPAPTPLGTSGLPNGLSGDEDFSSIADMDFSALL
SQISSSGQGGGGSGFSVDTSALLDLFSPSVTVPDMSLPDLDSSLASIQ
ELLSPQEPPRPPEAENSSPDSGKQLVHYTAQPLFLLDPGSVDTGSNDL
PVLFELGEGSYFSEGDGFAEDPTISLLTGSEPPKAKDPTVS
(Regular font for MS2, bold for p65
transactivation domain, Italics for HSF1
transactivation domain. Underlined sequences are
liner sequences and the underlined bolded
sequence is a nuclear localization signal)

### Example 2: CRISPR/gRNA-Directed Synergistic Activation Mediator (SAM) Induces Specific, Persistent and Robust Reactivation of the HIV-1 Latent Reservoir

The data presented herein explores the feasibility of this dCas9-SAM technology to activate HIV-1 long terminal repeat (LTR) promoter in HIV-1 latent cells. Two msgRNAs are identified which exhibited very robust and sustained reactivation of HIV-1 latent viruses. The target-specific compulsory reactivation leads to suicide of the HIV-1-infected cells. Such a specific and potent reactivation of HIV-1 latent reservoir may add a newer and more realistic alternative to the “shock and kill” strategy to potentially achieve a permanent cure of HIV/AIDS.

The materials and methods employed in these experiments are now described.

Plasmids and Cloning of sgRNA or msgRNAs Expression and EcoHIV-eLuc Vectors

The plasmids obtained for use in these experiments are: pMSCV-LTR-dCas9-VP64-BFP (Addgene, plasmid #46912; Gilbert et al., 2013, Cell 154:422-51), pHAGE TRE dCas9-VP64 (Addgene plasmid #50916; Kearns et al., 2014, Development 141:219-23), and lenti-MS2-P65-HSF1-Hygro (Addgene, plasmid #61426; Konermann et al., 2015, Nature 517:583-8).

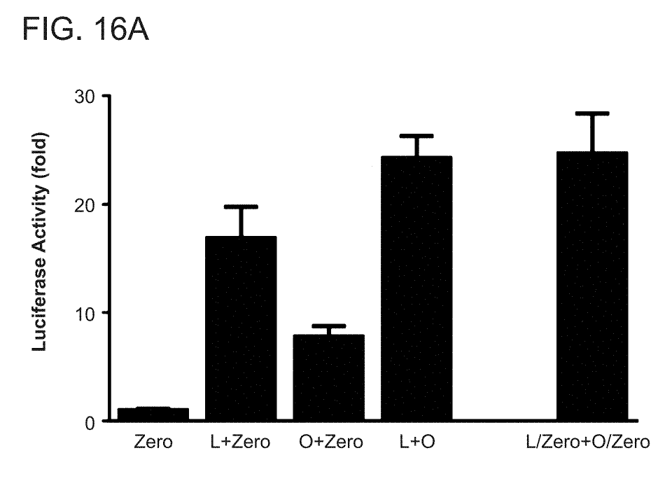

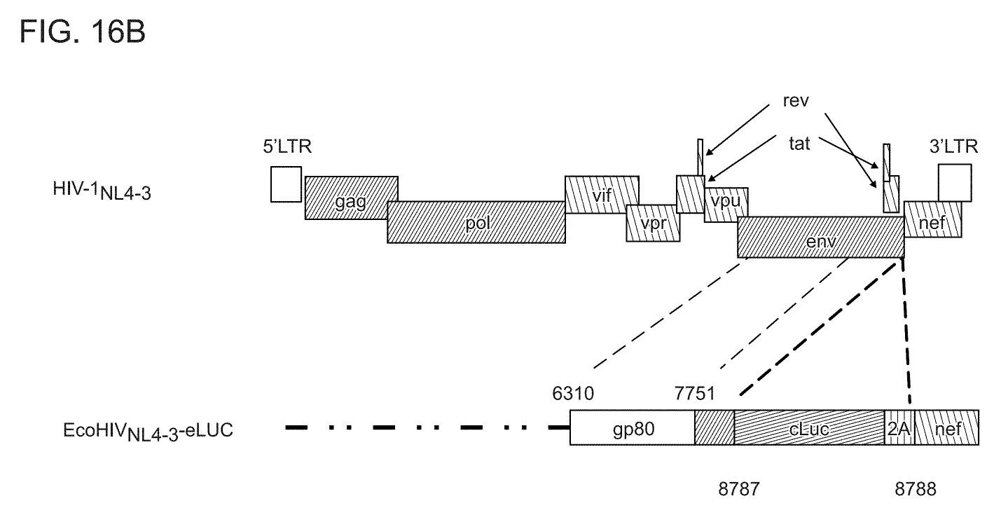

The lentiviral vector pLV-EF1α-dCas9-VP64-BFP was generated by cloning the BglII/XhoI-digested fragment of dCas9-VP64-BFP from retroviral vector pMSCV-LTR-dCas9-VP64-BFP (Addgene plasmid #46913; Gilbert et al., 2013, Cell 154:422-51) into lentiviral vector pLV-EF1α-Cas9-T2A-RFP via BamHI/SalI (Biosettia Inc). The pNL4-3-EcoHIV-eLuc vector was generated by infusion PCR (Heckman and Pease, 2007, Nat Protoc 2:924-32) with indicated primers (Table 4). The eLuc gene, a P2A self-cleaving peptide (Kim et al., 2011, PLoS One 6:e18556), and N-terminal of HIV-1 Nef in frame with HIV-1 splicing acceptor for HIV-1 Nef expression were amplified and then cloned into the BamHI and XhoI restriction sites of the HIV-1 proviral clone pNL4-3 (Adachi et al., 1986, J Virol 59:284-91). To generate pNL4-3-EcoHIV vector (FIG. 16), the coding region of gp120 in HIV-1 pNL4-3 was replaced with gp80 from ecotropic murine leukemia virus (MLV), which was PCR-amplified from pHCMV-EcoEnv (Addgene plasmid 15802; Sena-Esteves et al., 2004, J Virol Methods 122:131-9) following the engineering strategy previously published (Potash et al., 2005, PNAS 102:3760-5).

The seed sequences targeting the HIV-1 LTR (634 bp) were predicted by using the Broad Institute sgRNA designer tool for highly effective sgRNA design and MIT's CRISPR Design for the off-target prediction. Both sense and antisense target sequences using NGG as the PAM were described previously (Hu et al., 2014, PNAS 111:11461-6), from which 22 target sites with high score of cleaving efficiency and specificity against human genome were selected. A pair of oligonucleotides for each targeting site with 5′-CACC and 3′-AAAC overhang was obtained from AlphaDNA (Table 3). The overhang sequences are for the cloning of the seed sequences into the vector. For sgRNAs in the dCas9-single activator system, the target seed was cloned via modified BbsI sites into pKLV-WG lentiviral vector derived from pKLV-gRNA(BbsI)-Puro-2A-BFP lentiviral vector (Addgene #50946; Koike-Yusa et al., 2014, Nat Biotechnol 32:267-73). For msgRNAs in the dCas9-SAM system, the seed sequence was cloned via BsmBI sites into Lenti sgRNA(MS2)-zeo backbone (Addgene, Plasmid #61427; Konermann et al., 2015, Nature 517:583-8). The vectors were digested with BbsI or BsmBI, treated with Antarctic Phosphatase, and purified with a Quick nucleotide removal kit (Qiagen). Equal amount of complementary oligonucleotide was mixed in T4 polynucleotide kinase (PNK) buffer for annealing. These annealed seed pairs were phosphorylated with T4 PNK and ligated into the BbsI or BsmBI-digested lentiviral vector using T4 ligase. The ligation mixture was transformed into Stabl3 competent cells. Positive clones were identified by PCR screening and verified by Sanger sequencing using Flap or U6 primer (Table 4).

Cell Culture

TZM-bI reporter cell line from Dr. John C. Kappes, Dr Xiaoyun Wu and Tranzyme Inc (Derdyen et al., 2000, J Virol 74:8358-67), and J-Lat full length clone from Dr. Eric Verdin (Jordan et al., 2003, EMBO J 22:1868-77) were obtained through the NIH AIDS Reagent Program, Division of AIDS, NIAID, NIH. CHME5/HIV fetal microglia cell line and Jurkat-derived T cell line 2D10, 3C9, E4 were donated from Dr. Jonathan Karn (Jadlowsky et al., 2014, Mol Cell Biol 34:1911-28; Mbonye and Karn, 2014, Virol 454-455:328-39; Wires et al., 2012, J Neurovirol 18:400-10). TZM-bI and CHME5 cells were cultured in Dulbecco's minimal essential medium high glucose supplemented with 10% heat-inactivated fetal bovine serum (FBS) and 1% penicillin/streptomycin. J-Lat, 2D10, 3C9, and E4 T cells were cultured in RPMI1640 containing 2.0 mM L-glutamine, 10% FBS and 1% penicillin/streptomycin.

Lentivirus and Retrovirus Packaging and Infection

All recombinant lentiviruses or retroviruses were produced after calcium phosphate-mediated transient transfection of related vectors according to standard protocols. Briefly, HEK293T cells were cotransfected with the lentiviral transfer vector (10 μg for gRNA, 15 μg for others), lentiviral packaging vectors pRSV-REV (3 μg) and pMDLg/pRRE (8 μg), and vesicular stomatitis virus G glycoprotein (VSVG) expression vector pMD2G (5 μg). For retrovirus, the GP2-293 cells were cotransfected with retroviral vector pMSCV-LTR-dCas9-VP64-BFP (15 μg) and pMD2G (15 μg). The viruses were collected from the culture supernatant on days 2 and 3 post-transfection, concentrated by ultracentrifugation for 2 hours at 25,000 rpm, and then resuspended in phosphate-buffered saline (PBS). Virus titer determination was performed by infecting HEK293T cells with serial diluted lentiviruses and counting the number of fluorescent protein-expressing cells 48 hours post-infection under fluorescent microscopy. For a typical preparation, the titer was approximately 4-10×10⁸ IU/ml for gRNA and 4-10×10⁷ IU/ml for others. The experimental cells were infected at 10 MOI of indicated lentivirus in the presence of polybrene (8 μg/ml) by centrifugation at room temperature at 400 g for 2 hours. After infection, cells were cultured for next experiments or drug selection.

Stable Cell Lines

TZM-bI, CHME5 or HEK293T cells were seeded in 24-well plates at 2×10⁴ cells/well and transduced at 10 MOI with pMSCV-dCas9-BFP (Puromycin) and Lenti-MS2-p65-HSF1 (hygromycin). After 2 days, cells were subcultured in 6-well-plate and selected with puromycin (2 μg/ml) and hygromycin (200 μg/ml). After two weeks of selection culture, cells were seeded in 24-well plates at 2×10⁴ cells/well, and infected with indicated msgRNA lentivirus (10 MOI). After 2 days, cells were subcultured in 6-well-plate and selected with triple antibiotics: puromycin (0.5 μg/ml), Hygromycin (100 μg/ml) and Zeomycin (100 μg/ml).

Firefly-Luciferase Reporter Assay

Cells were cultured in a 96-well plate and transfected or transduced with indicated vectors. To examine the eLuc reporter activity, the cell lysate was prepared using the ONE-Glo luciferase assay system (Promega) and luminescence was measured in a 2104 ENVISION® Multilabel Reader (PerkinElmer). Representative results were presented as mean±SEM of 4-6 independent samples.

EGFP Flow Cytometry

Cells were trypsinized (for CHME5) or collected (for suspension T cell lines), washed with PBS and fixed in 2% paraformaldehyde for 10 minutes at room temperature. Then, cells were washed twice with PBS and analyzed using a Guava EasyCyte Mini flow cytometer (Guava Technologies).

Cell Growth/Proliferation Assay

The cell growth/proliferation was determined by the trypan blue exclusion hemocytometry, and CELLTITER-GLO luminescence viability assay (Promega). The CELLTITER-GLO luminescent cell viability assay is a homogeneous and sensitive method to quantitate ATP generated by metabolically active cells that associates with the number of viable cells. Briefly, cells were cultured in sterile 96-well plates for indicated period and treated with 100 μl of CELLTITER-GLO reagent for 10 min at room temperature. The luminescence in each well was measured in a 2104 EnVision® Multilabel Reader (PerkinElmer).

Cell Apoptosis Assay

The dCas9-VPH stably-expressing CHME5 cells were seeded in 96-well-plate (2,000 cells/well) and infected with indicated msgRNA lentiviruses. At 2 days post-infection (dpi), the caspase-3/7 activities were examined using a CASPASE-GLO® 3/7 luminescence assay kit (Promega, Madison, Wis.) according to the product manual.

Statistical Analysis

The quantitative data represented mean±standard error from 3-5 independent experiments, and were evaluated by ANOVA and Fisher's LSD multiple comparison test, or unpaired Student's t-test in some cases. A statistically significant difference was marked as * and ** for p value <0.05 and 0.01 respectively

The results of the experiments are now described.

Bioinformatic Screening of Effective sgRNAs that Guide dCas9-Single Activator to the HIV-1 LTR Promoter

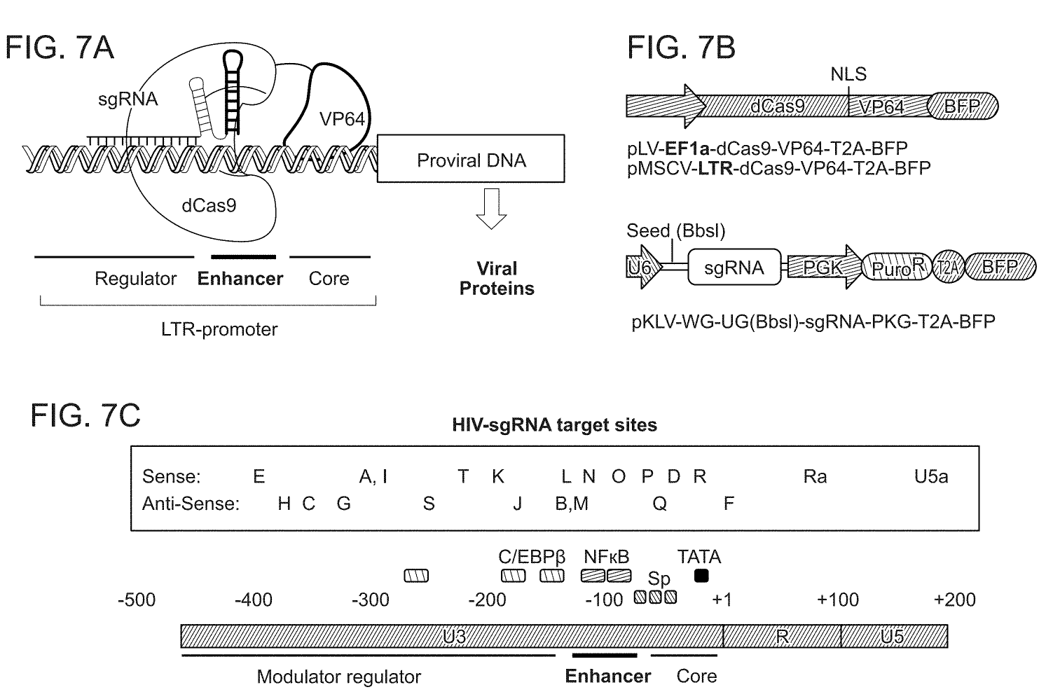

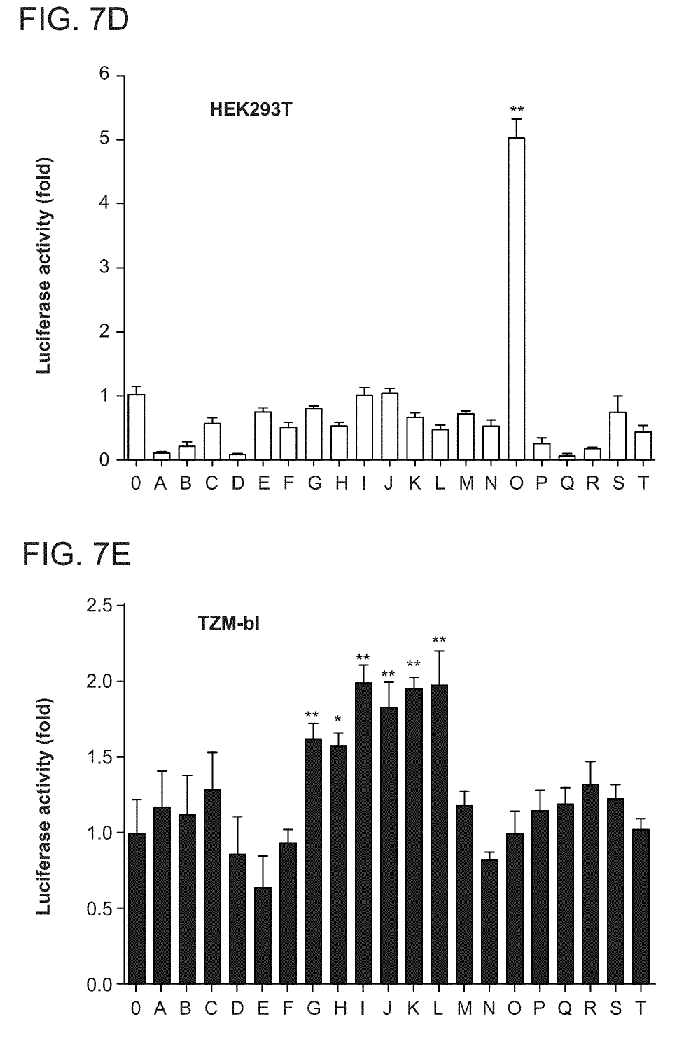

The dCas9 has been widely explored via fusion with transcriptional activators (e.g. VP64, p65) or repressor (e.g. KRAB) (Agne et al., 2014, ACS Synth Biol 3:986-9; Maeder et al., 2013, Nat Methods, 10:977-9; Gilbert et al., 2013, Cell 154:422-51; Cheng et al., 2013, Cell Res 23:1163-71) to regulate transcriptional activation or repression of cellular genes through target gene-specific sgRNAs. To provide a similar proof of concept in viral replication/infection regulation (FIG. 7A, FIG. 7B), 20 sgRNA target sites were designed within the HIV-1 promoter (LTR-U3) region using Cas9-sgRNA design tools for best scores of high efficiency and high specificity (FIG. 7C). These seed sequences were cloned into a sgRNA expression lentiviral vector (FIG. 7B). The EcoHIV-firefly-luciferase (eLuc) reporter assay was utilized for the sensitive, easy screening and HIV-1 naïve virus similarity. One of these sgRNA lentiviral vectors was contransfected with the dCas9-VP64 lentiviral vector plus a pNL4-3-EcoHIV-eLuc reporter (1:20 to ensure the expression of dCas9-VP64/sgRNA in each reporter-expressing cells) in HEK293T cells, and measured the production of the EcoHIV reporter virus using OneGlo eLuc luminescence assay at 2 days post-transfection. Only LTR-O sgRNA induced a 5-fold increase in reporter activation but the majority of these sgRNAs exhibited a marginal change in reporter activities (FIG. 7D). To determine if the dCas9-VP64/LTR-sgRNA system effectively reactivates the latent HIV-1 provirus, a TZM-bI cell line that contains an integrated LTR-eLuc reporter gene, which is a well-established model to study HIV-1 latency reactivation, was utilized (Derdyen et al., 2000, J Virol 74:8358-67). Through cotransfection of dCas9-VP64 with indicated sgRNA expressing vectors via lipofectamine 3000 (to help maximize transfection efficiency), marginal levels of reactivation (1.5-2 fold) in a few sgRNAs LTR-G to LTR-L were observed (FIG. 7E). These data suggest that most of the selected LTR-sgRNAs with the dCas9-single activator system do not remarkably activate HIV-1 LTR promoter, with LTR-I, J, K, L showing highest, but marginal reactivation of the HIV-1 LTR latent reporter.

Screening of Effective msgRNAs that Guide dCas9-SAM to Activate HIV-1 LTR Promoter

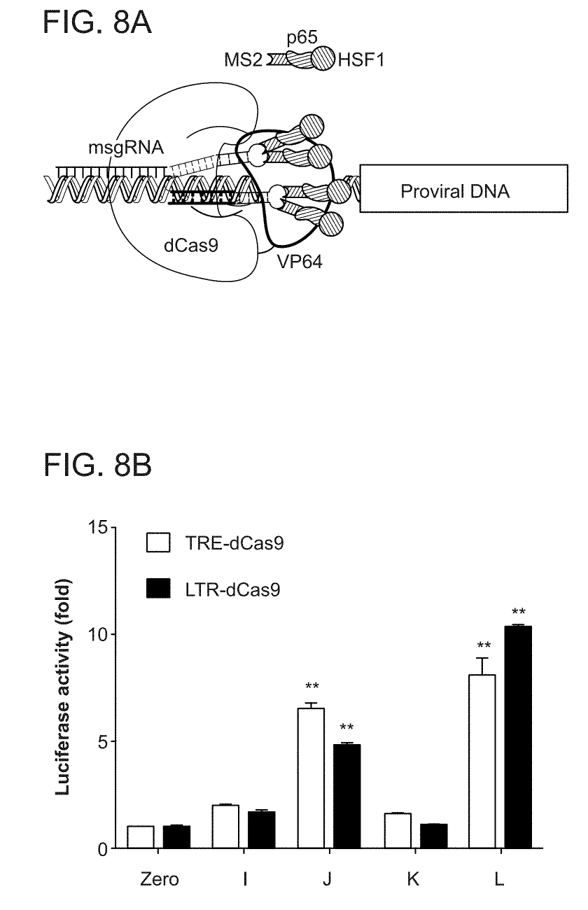

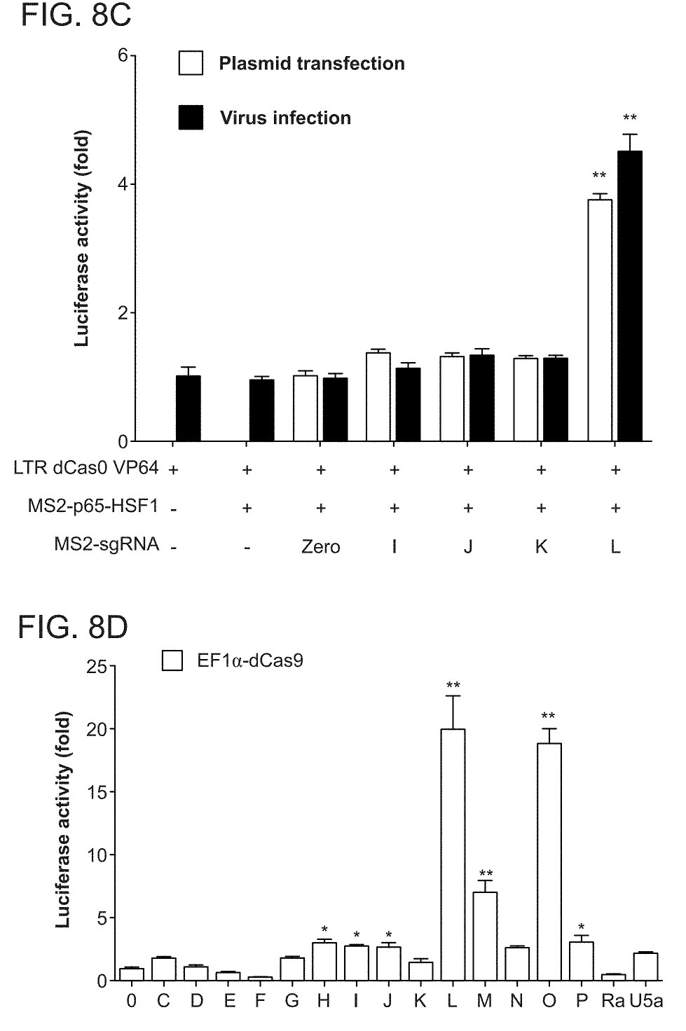

In cellular genes, a complex of multiple transcriptional activators showed much stronger activation than the single dCas9-transcription factor itself (Konermann et al., 2015, Nature 517:583-8; Chavez et al., 2015, Nat Methods 12:326-8; Hilton et al., 2015, Nat Biotechnol 33:510-7). Thus, the newly-developed MS2-mediated SAM system was applied (FIG. 8A), which combined dCas9-VP64 with MS2-p65-HSF1 clustered trans-activators, designated dCas9-VPH (Konermann et al., 2015, Nature 517:583-8). First feasibility of dCas9-VPH in facilitating LTR promoter activation was tested by cloning the seed sequence of the effective sgRNAs LTR-I, J, K, L into the msgRNA-expressing lentiviral vector. This uses the same sgRNA expression cassette incorporated with 2 hairpin aptamers selectively bound to dimerized MS2-fusion proteins at the sgRNA tetraloop and stem loop 2. Such a design allows the recruitment of 4 copies of MS2-fusion protein to a single msgRNA. By cotransfection of pLV-EF1a-dCas9-VP64-BFP/pLV-MS2-p65-HSF1 (dCas9-VPH), and pNL4-3-EcoHIV-eLuc vectors with each of msgRNAs LTR-I, J, K and L in HEK293T cells, it was observed that LTR-J and L increased the EcoHIV eLuc reporter activity by 6-7 fold compared to the empty msgRNA LTR-Zero control, but msgRNA LTR-I and K showed no noticeable activation of the EcoHIV-reporter (FIG. 8B). This result was further confirmed by inducible expression of dCas9-VP64 to replace the constitutive dCas9-VP64 (FIG. 8B). To determine if dCas9-VPH/msgRNAs induced a better reactivation of HIV-1 latency, HIV-latent TZM-bI cells was cotransfected or cotransduced with dCas9-VPH and indicated msgRNAs (FIG. 8C) and the latency-reversing capability was measured. The msgRNA LTR-L expression by Lipofectamine 3000 transfection effectively induced a 3.7-fold reactivation of latent HIV-1 LTR eLuc reporter activity and lentiviral transduction of these components for msgRNA-L in TZM-bI cells caused a 4.5-fold increase in HIV-1 LTR-promoter activity (FIG. 8C). These data suggest that dCas9-VPH exhibits better reactivation than dCas9-VP64 alone in HIV-1 latent reporter, which also relies on gene delivery efficiency. The absence of reactivation by msgRNA LTR-J in TZM-bI cells in contrast to EcoHIV-eLuc reporter in HEK293T cells reflected the fact that one mismatch was found by PCR cloning and Sanger sequencing in the target sequence of integrated LTR in TZM-bI cells (Hu et al., 2014, PNAS 111:11461-6).

To further screen for the best msgRNAs with maximal reactivation efficiency within the HIV-1 LTR, the seed sequences of the sgRNAs were cloned as described above and two additional seed sequences targeting the R and U5 regions were cloned (Table 3), which also contain an enhancer for LTR promoter regulation. The seed sequences were transiently co-transfected with dCas9-VPH into HEK293T cells in the presence of a pNL4-3-EcoHIV-eLuc reporter vector. Twelve of 16 designed msgRNAs increased HIV-eLuc activity but only L and O substantially up-regulated the eLuc reporter expression by up to 20-fold at 2 days after transfection (FIG. 8D). Taken together, these sets of data suggest that the MS2-mediated dCas9-SAM system dramatically improves the efficiency of promoting HIV-1 transcription, and msgRNAs LTR-L and O are the most effective among the tested msgRNAs.

Robust Reactivation by LTR-L/O msgRNAs in HIV-1 Latent TZM-bI Cells

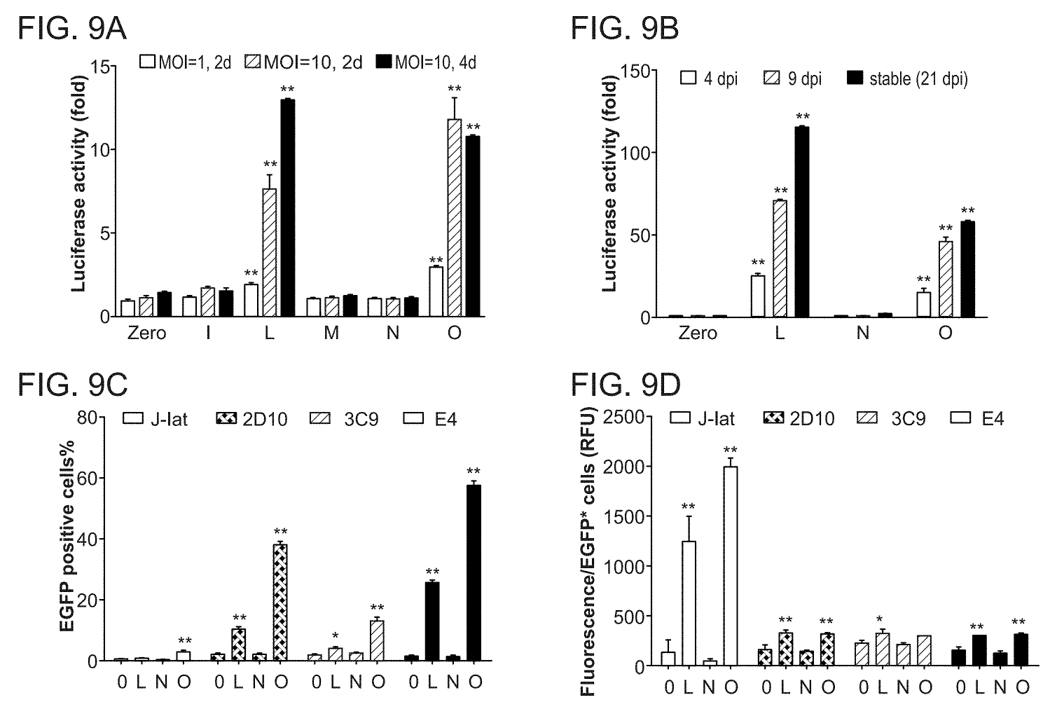

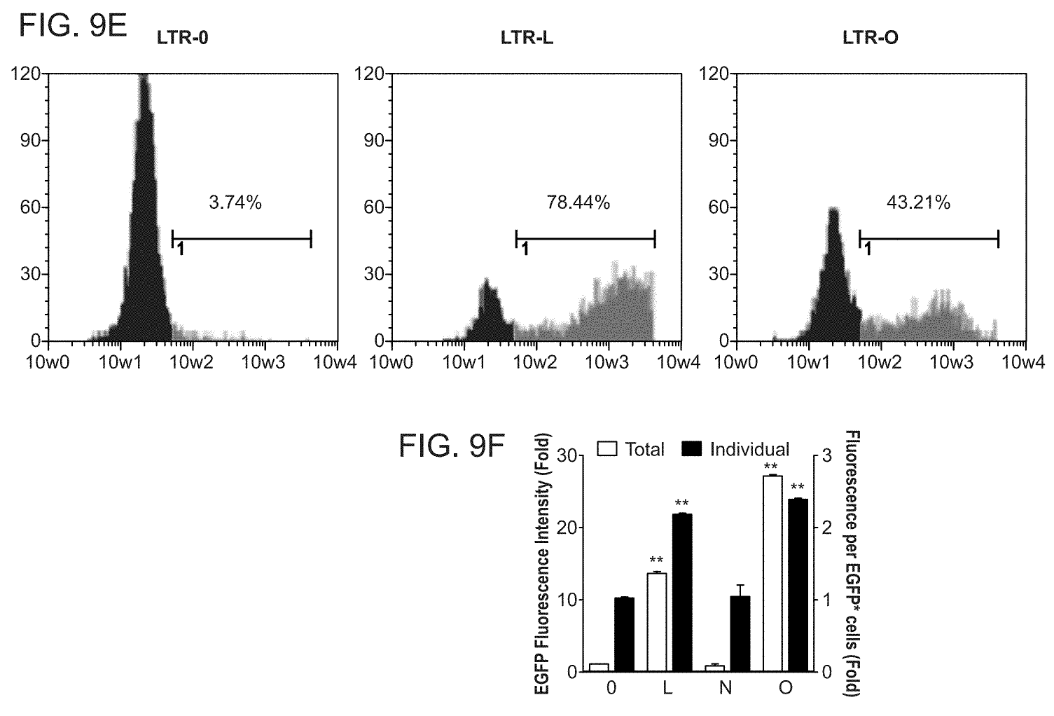

To examine the latency-reversing efficiency of the identified msgRNAs LTR-L and O in TZM-bI HIV-1 latent cells, a dCas9-VPH-expressing stable cell line was established by lentivirus infection and double selection with puromycin for dCas9-VP64 and hygromycin for MS2-p65-HSF1 followed by lentiviral transduction with indicated msgRNAs. Such a strategy ensured a higher feasibility of expressing all three genes in a single cell and thus increased the gene delivery efficiency in TZM-bI cells. msgRNA LTR-L and O induced robust activation of LTR-eLuc reporter in a dose- and time-dependent manner, while msgRNA-I, M, N showed no effect (FIG. 9A-FIG. 9B). At 21 dpi, the reactivation efficiency reached 115-fold (FIG. 9B). The effective msgRNA LTR-L and O are consistent with the finding above in HEK293T cells using transiently transfected EcoHIV-eLuc reporter. These data suggest that the latency-reversing potency by LTR-L and O is specific, accumulative and robust.

Preclinical Application of LTR-L/O msgRNAs-Induced Reactivation in HIV-1 Latently-Infected Cell Lines

Memory T cells are the best-studied HIV-1 latent reservoir. To explore the feasibility of dCas9-SAM in reactivating HIV-1 provirus in latent T cells, an HIV-1 EGFP reporter assay was performed using several HIV-1 latency T cell lines, including J-Lat (Jordan et al., 2003, EMBO J 22:1868-77), 2D10, 3C9 and E4 (Hu et al., 2014, PNAS 111:11461-6; Jadlowsky et al., 2014, Mol Cell Biol 34:1911-28). Co-transduction of all dCas9-SAM components via a lentiviral gene delivery system resulted in a dramatic increase in the number of reactivated EGFP cells (FIG. 9C) and relative fluorescence intensity per EGFP⁺ cells (FIG. 9D). Various reactivation levels in different T cell lines may result from different transduction efficiency and perhaps transcriptional mechanisms (Jadlowsky et al., 2014, Mol Cell Biol 34:1911-28; Mbonye and Karn, 2014, Virol 454-455:328-39).

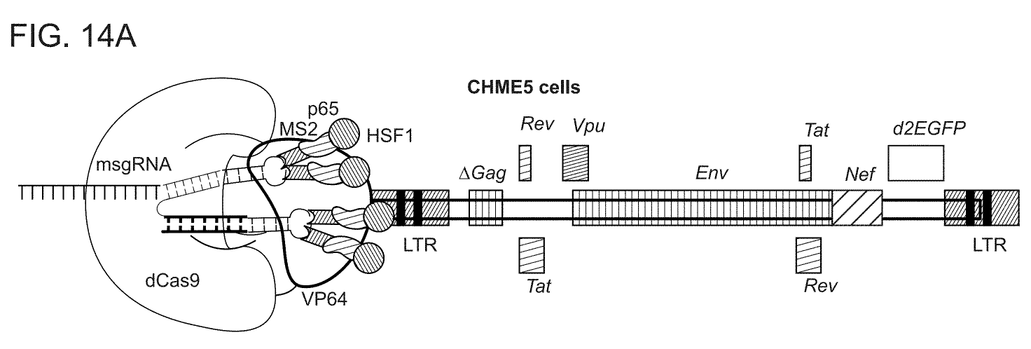

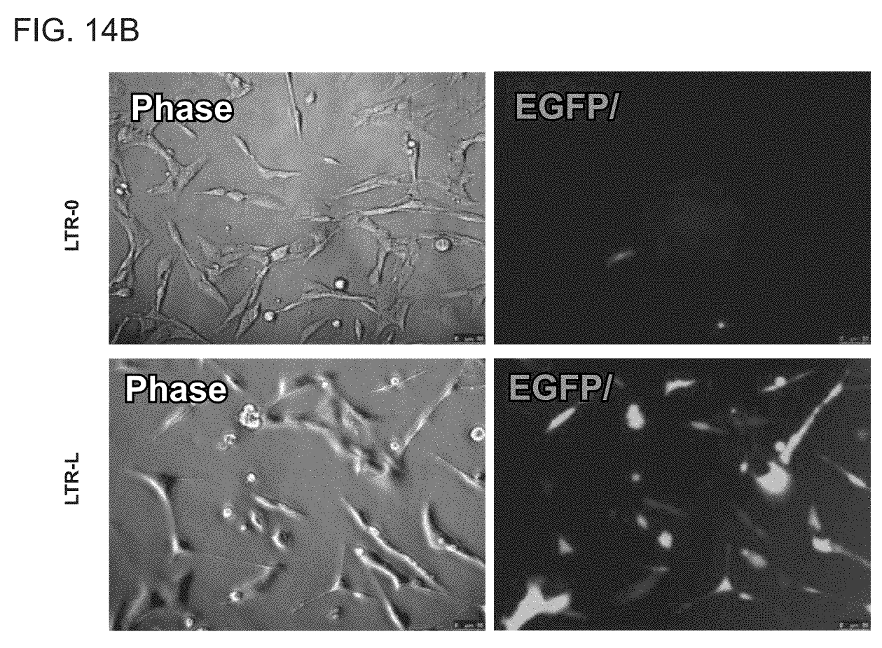

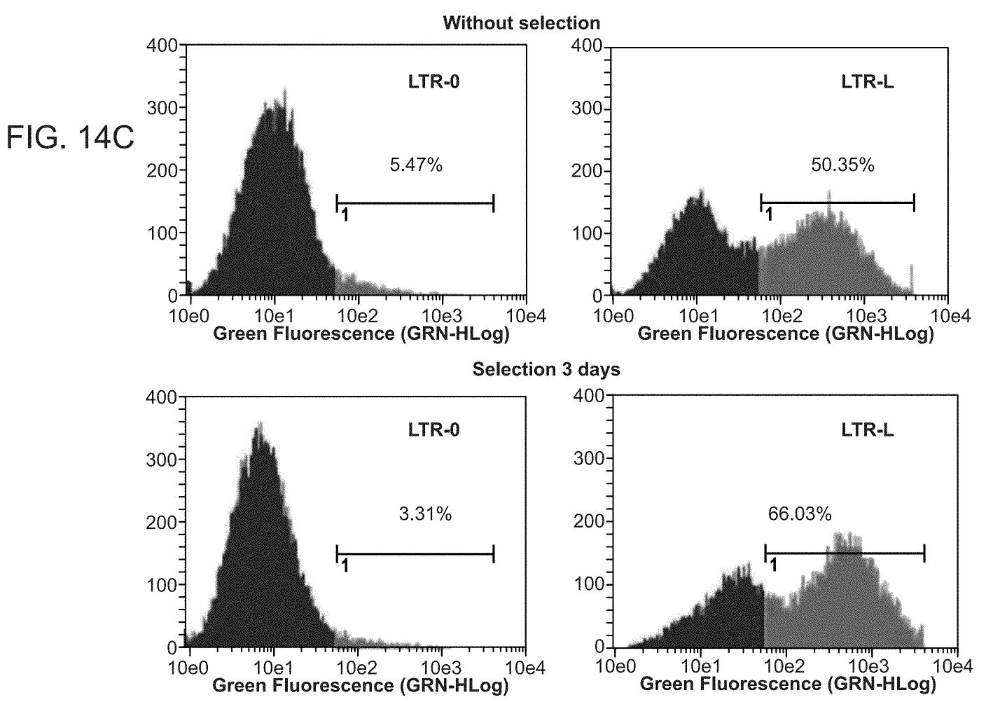

To evaluate the latency-reversing properties of the dCas9-SAM system in a brain latent reservoir, similar experiments were conducted using a CHME5 microglial cell line, a well-established model to study the latent infection and reactivation in NeuroAIDS (FIG. 14A) (Wires et al., 2012, J Neurovirol 18:400-10). After cotransduction of dCas9-VPH and indicated msgRNAs, both the percentage (FIG. 9E) and the intensity (FIG. 9F) of EGFP expression were increased dramatically in LTR-L and O. A higher reactivation efficiency in CHME5 cells than T cells may be attributed to a higher efficiency of gene delivery in CHME5 cells.

### Sustained/Increasing Reactivation and Virus-Induced Suicide Death

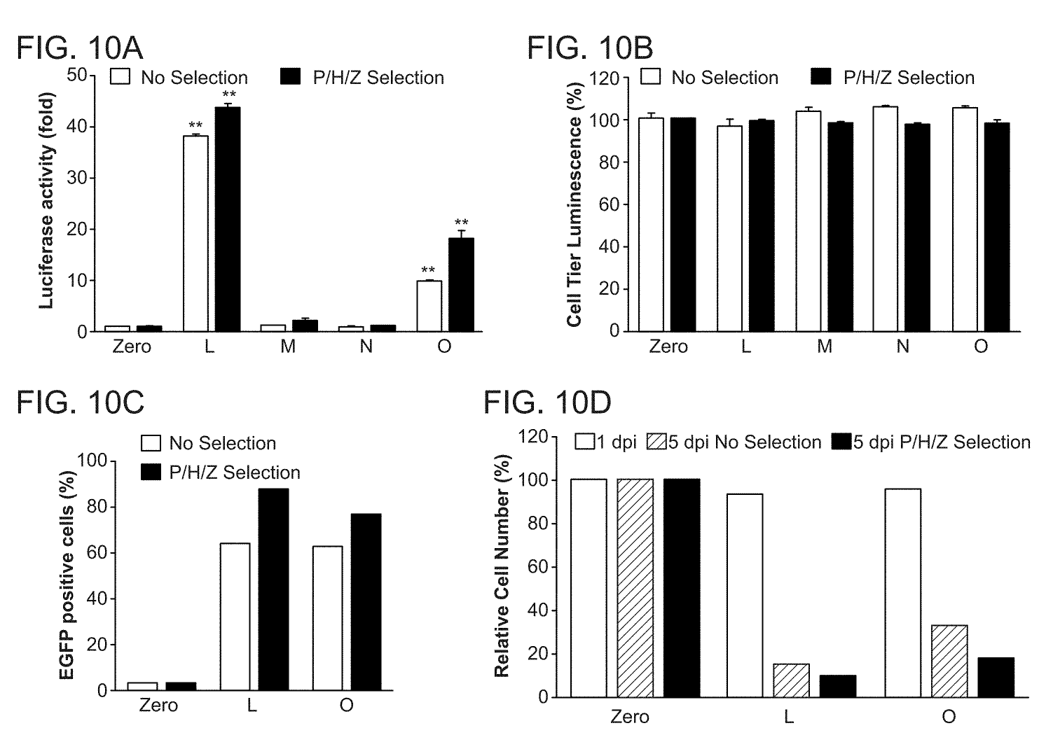

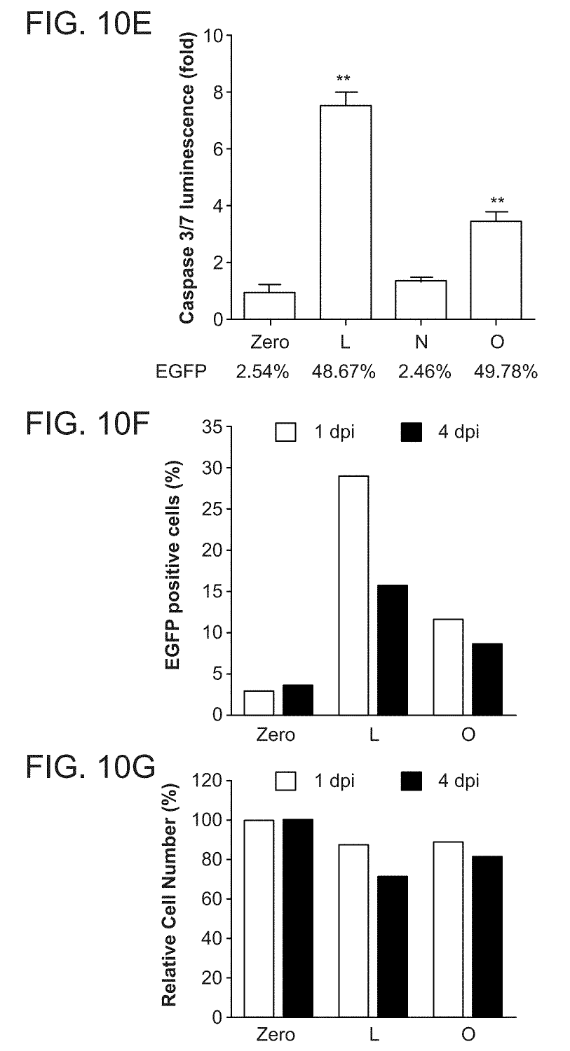

As shown in FIG. 9B, a lentivirus-mediated dCas9-VPH/msgRNA system is capable of sustaining HIV-1 reactivation over the time course (21 days) of the experiments. To further pursue this, the infected cells were passaged in the presence or absence of antibiotic selection (to clear out any uninfected cells). TZM-bI cells retained the growth capacity and the continuous LTR activation even after 3 passages (FIG. 10A). There was no significant difference in the cell proliferation/survival (FIG. 10B) between the activation-effective groups (LTR-L and O) and the activation-ineffective groups (LTR-Zero, M, N). Triple antibiotic selection enriched the infected cells and increased the activation efficiency as expected, but did not kill a significant number of cells (<2-7%) due to high infection efficiency. This finding implies that the LTR-eLuc reporter system in TZM-bI cells does not produce toxic viral proteins as designed.

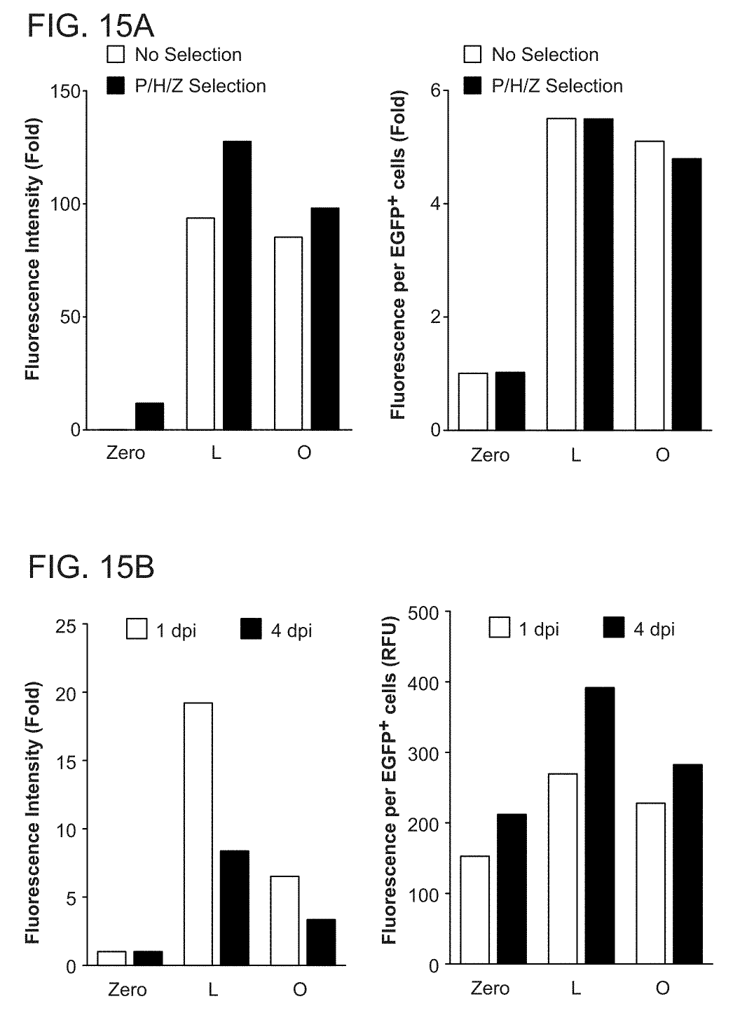

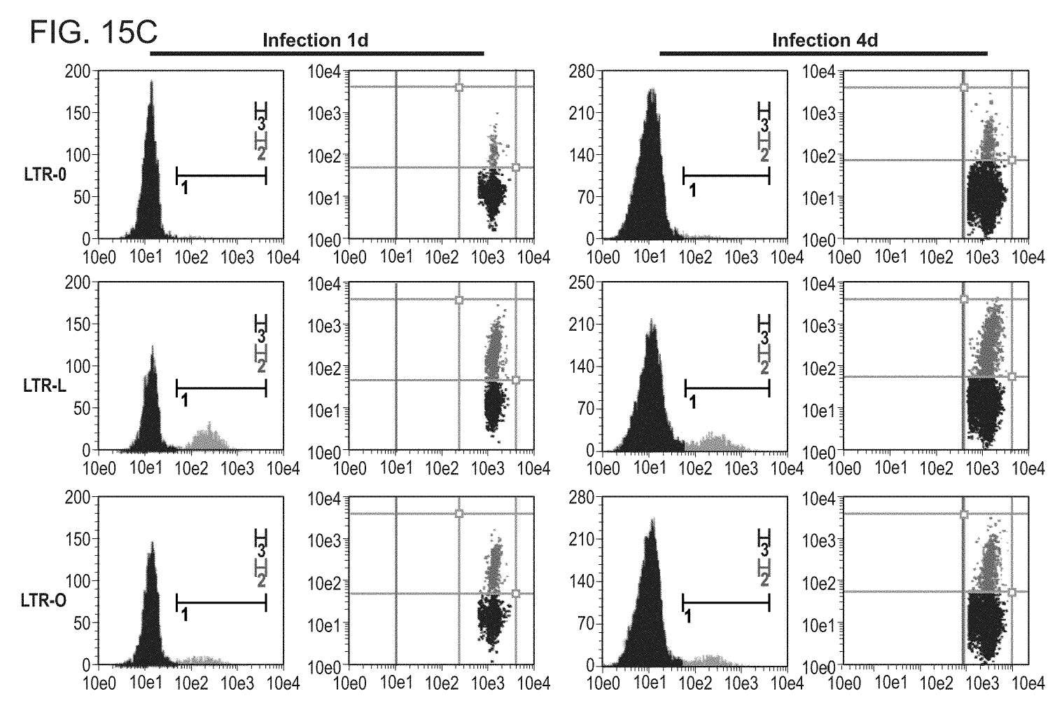

In contrast, the CHME5 and 2D10 cells were unable to be continuously passaged due to continuous apoptosis of the latency-reversed cells via toxic viral protein buildup (FIG. 10C-FIG. 10G). Both CHME5 and 2D10 cell lines contain the HIV-1 genome with a deletion in the partial Gag domain and full Pol domain and an insertion of the d2EGFP encoding sequence near the 3′-LTR (Jadlowsky et al., 2014, Mol Cell Biol 34:1911-28; Wires et al., 2012, J Neurovirol 18:400-10). Both generate toxic viral proteins such as Tat, Vpu, Rev, Env, etc (FIG. 14A). For the first passage of CHME5 cells at 5 dpi, the reactivation percentage (FIG. 10C, FIG. 14B-FIG. 14C), total fluorescence intensity and individual fluorescence intensity (FIG. 15A) for the EGFP reporter remained high in LTR-L and O groups while comparing to that at 1 dpi (FIG. 9F). Interestingly, the sustained reactivation by both LTR-L and O induced robust cell death in more than 80% of infected cells at 5 dpi (FIG. 10D), corresponding to the high efficiency of lentivirus infection. Similarly, triple antibiotic selection enriched the infected cells and increased the reactivation efficiency and cell death moderately as expected (FIG. 10C-FIG. 10D). When the LTR-Zero control cells grew to confluence for passage, the number of cells with effective reactivation by LTR-L or O was too small to be analyzed with flow cytometry. When the LTR-Zero control cells reached the third passage, the reactivated cells in LTR-L or O groups were completely lost as visualized under fluorescent microscopy. A caspase 3/7 apoptotic assay verified a significant increase in the number of apoptotic cells after LTR-L and O induced reactivation (FIG. 10E). Interestingly, LTR-O and LTR-L induced similar levels of HIV-1 provirus reactivation (FIG. 10C, FIG. 15A), but the apoptotic cell death was significantly less in LTR-O than that in LTR-L (FIG. 10D-FIG. 10E), suggesting that LTR-L and O may affect different patterns of viral protein expression.

For 2D10 cells, the viral protein-induced cell death was less (10-28%) compared to CHME5 cells probably due to lower reactivation efficiency (FIG. 9C, FIG. 10F-FIG. 10G, and FIG. 15B-FIG. 15C). The percentage and total fluorescence intensity of the reactivated EGFP was reduced at 4 dpi from that at 1 dpi due to viral protein-induced cell death (FIG. 10G, FIG. 15B-FIG. 15C). However, the fluorescence intensity in single reactivated cells remained elevated at 4 dpi (FIG. 15B), indicating sustained reactivation of latent HIV-1 EGFP reporter.

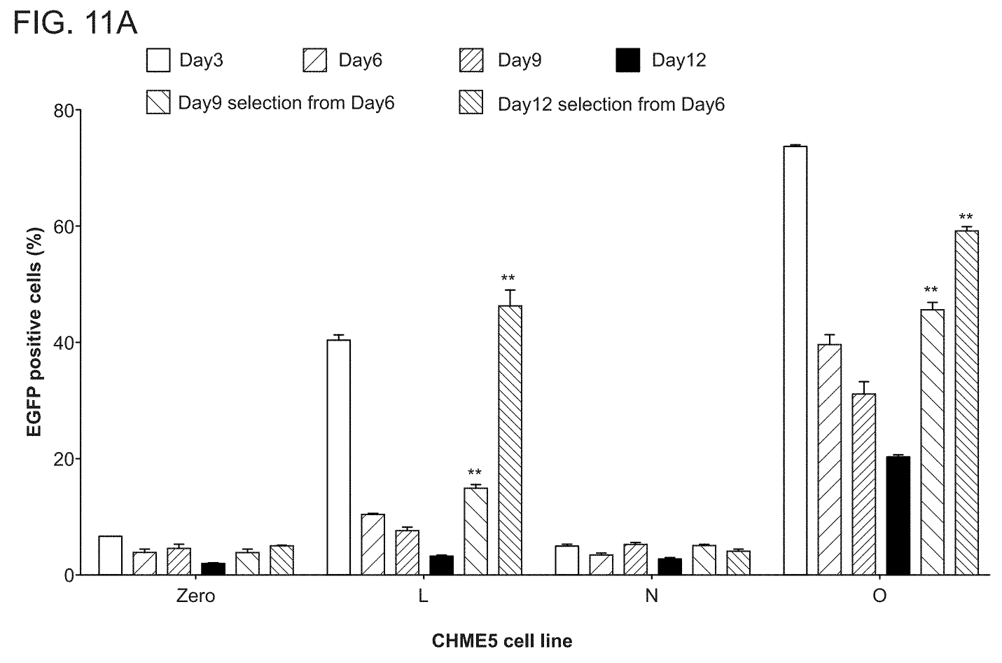

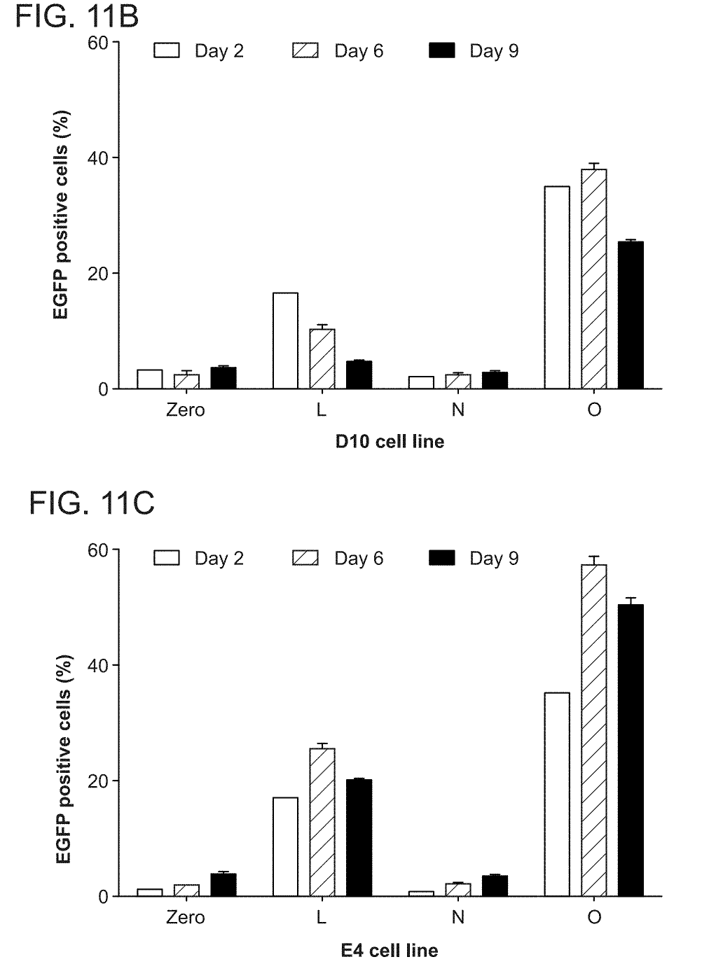

During continuous culture, the numbers of reactivated CHME5 (FIG. 11A) and Jurket-derived T cells (FIG. 11B-FIG. 11C) were gradually reduced due to cell death, which is in contrast to the sustained and increasing reactivation of LTR-luciferase reporter in TZM-bI cells (FIG. 9B). Again, the declination of reactivation was slower in LTR-O than LTR-L in CHME5, 1D10 and E4 cells (FIG. 11A-FIG. 11C), implying that LTR-O msgRNA may promote the expression of some anti-apoptotic viral proteins or probably other cellular proteins in these cell lines. This was further evidenced in J-lat and 3C9 cell lines, in which the majority of cells died at the first passage after introducing LTR-L or O by lentiviral transduction. These data suggest that the provirus reactivation response and mechanisms varied with different latent cell lines, which is consistent with previous reports (Jadlowsky et al., 2014, Mol Cell Biol 34:1911-28; Mbonye and Karn, 2014, Virol 454-455:328-39). Selective and sustained reactivation of the latent HIV-1 reservoir induces suicide death via the toxic viral proteins but does not affect the survival of neighboring non-reactivated cells.

The Advantage of dCas9-SAM-Induced LTR Reactivation Over Latency-Reversing Agent

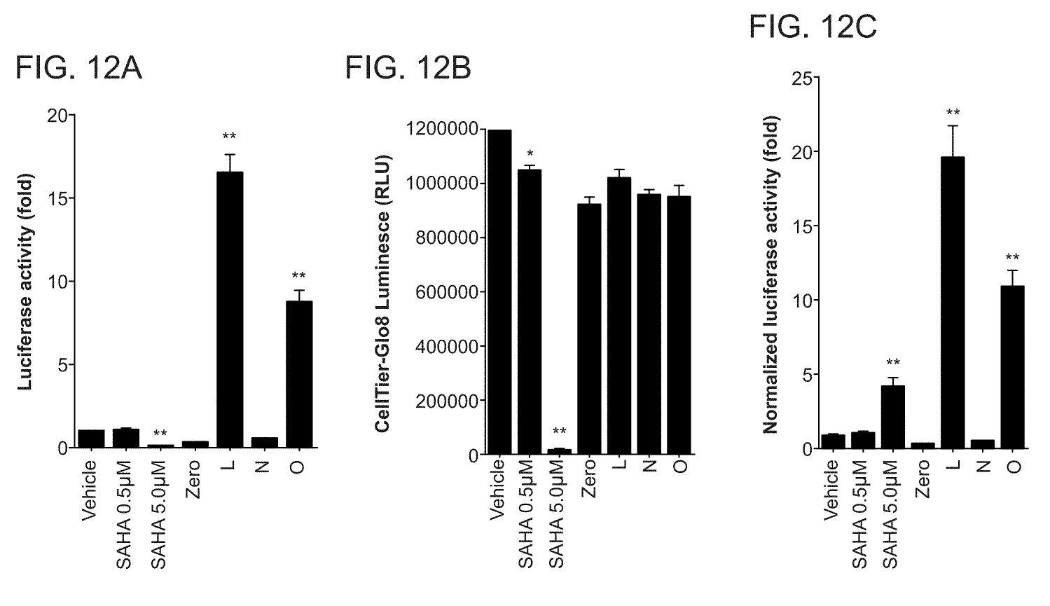

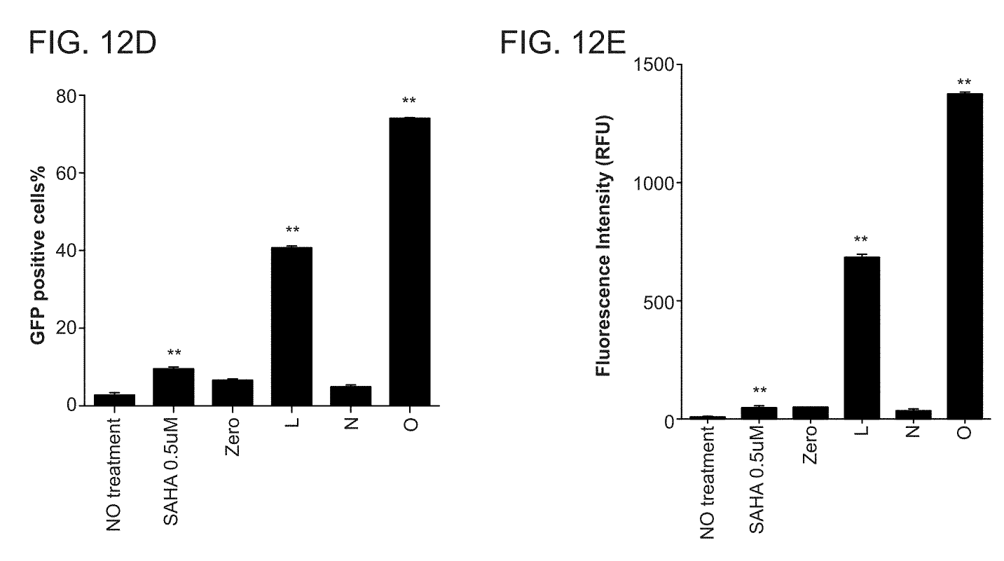

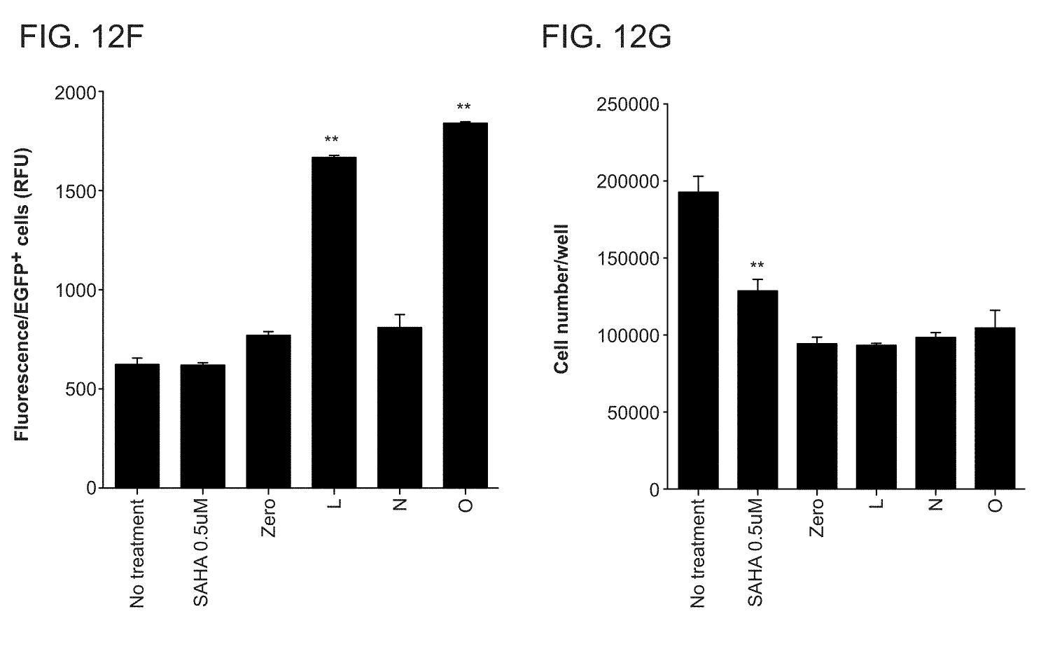

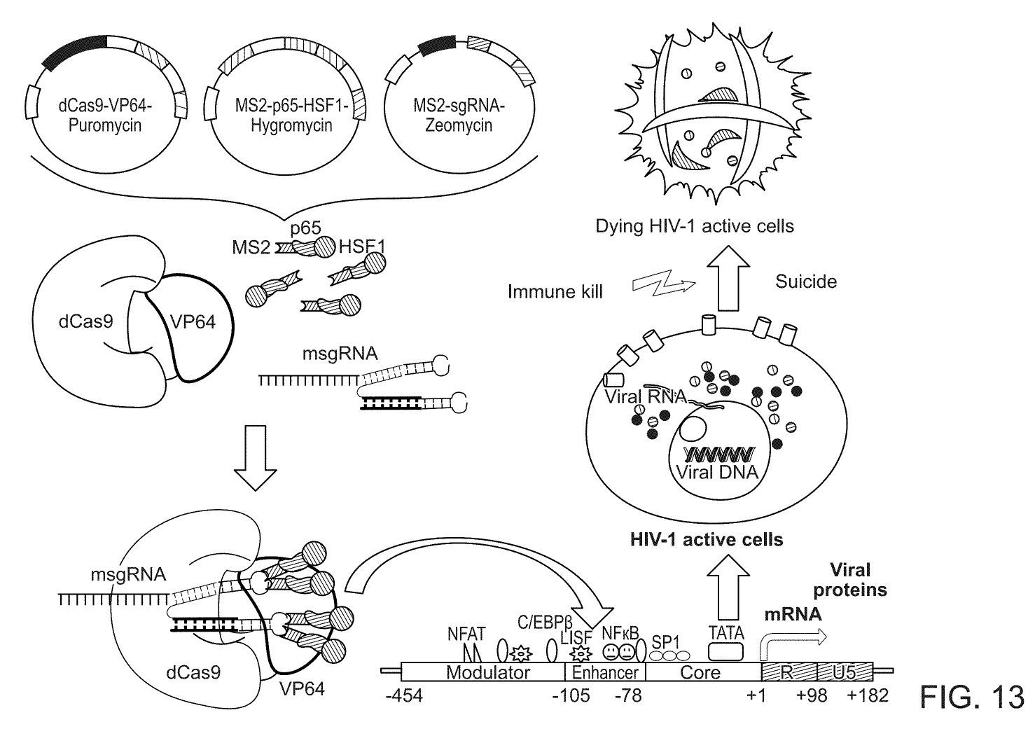

Several latency-reversing agents have been identified to “shock” the HIV-1 latent reservoir (Wei et al., 2014, PLoS Pathog 10:e1004071; Lucera et al., 2014, J Virol 88:10803-12; Spivak et al., Clin Infect Dis 58:883-90; Xing and Silicano, 2013, Drug Discov Today 18:541-51). The HDAC inhibitor SAHA is widely used in clinical practice and holds a promising application in HIV-1 cure with the “shock and kill” strategy (Ramakrisnan et al., 2015, AIDS Res Hum Retroviruses 31:137-41; Olesen et al., 2015, J Virol 89:10176-89). However, HDAC inhibition does not activate HIV-1 latency in some cases due to different epigenetic mechanisms (White et al., 2015, Antiviral Res 123:78-85; Ramakrisnan et al., 2015, AIDS Res Hum Retroviruses 31:137-41). SAHA was chosen as a representative example to determine if the dCas9-SAM system functions better than currently used latency reversing agents in terms of cellular response, efficiency, specificity and persistence. TZM-bI cells were selected because the integrated LTR promoter is poorly responsive to SAHA (FIG. 12A) (Klase et al., 2014, PLoS Pathog 10:e1003997). A higher dose (500 nM) of SAHA did not activate the LTR-eLuc reporter, while a super-high dose (5,000 nM) induced 4-fold reactivation, but killed all the cells (FIG. 12B-FIG. 12C). In contrast, the dCas9-VPH/msgRNA LTR-L and O induced robust reactivation but did not significantly cause any cell death as compared with LTR-Zero (FIG. 12A-FIG. 12C). The reduced cell viability in the LTR-Zero group as compared to the vehicle control group may reflect the growth inhibition by a higher titer of lentivirus. Interestingly, continuous passaging and triple antibiotic selection produced an increasing reactivation up to 115-fold at 21 dpi in TZM-bI cells (FIG. 9B). To further evaluate a better reactivation of latent provirus by the dCas9-VPH/msgRNA, the effect of SAHA in CHME5 cells was compared to TZM-bI cells. Similar to TZM-bI cells, high dose (500 nM) of SAHA induced a marginal increase in HIV-1 provirus reactivation (FIG. 12D-FIG. 12G). However, the dCas9-VPH/msgRNA LTR-L and O induced robust reactivation at 4 dpi in CHME5 cells (FIG. 12D-G). These data suggest that dCas9-SAM technology induces a compulsory reactivation independent of the latency mechanism, and exhibits better reactivation efficiency and specificity than SAHA.

### Additive Activation of HIV-1 Virus by Multiplex LTR-msgRNAs

As described above, 2 msgRNAs with the highest efficiency targeting −145 and −92 bp from the transcriptional start site (TSS) within the HIV-1 LTR promoter were identified (Table 3). It was hypothesized that multiplex msgRNAs may produce synergistic and/or additive action. To test this, msgRNAs LTR-L and -O were coexpressed in dCas9-VPH expressing HEK293T cells and a EcoHIV-eLuc reporter assay was performed. As shown in FIG. 16, co-transfection of msgRNA LTR-L and -O induced additive activation of the EcoHIV-eLuc reporter. These data suggest that multiplex msgRNAs enriched transcriptional activators within the LTR promoter regions and ultimately increased the reactivation efficiency.

msgRNAs Induce Sustained Reactivation of the HIV-1 Latent Provirus Resulting in Suicide Death of HIV-1 Infected Cells

As HIV-1 latent cellular reservoirs persist even in the cART era, stalling the road to a permanent cure for HIV-1 infection. As of now, complete elimination of HIV-1 latent reservoirs from the whole body remains a big challenge. Two promising strategies to cure HIV/AIDS have been developed: proviral genome eradication (Hu et al., 2014, PNAS 111:11461-6) and latency-reversal in reservoir cells (Sgarbanti and Battistini, 2014, Curr Opin Virol 3:394-401; Halper-Stromberg et al., 2014, Cell 158:989-99). The latter strategy, well known as “shock and kill” (also dubbed “kick and kill” or “reactivation and elimination”), is widely employed to wake up the dormant proviruses for subsequent clearance of latently-infected cells by viral cytotoxicity and/or host immune defense mechanisms (Wei et al., 2014, PLoS Pathog 10:e1004071; Lucera et al., 2014, J Virol 88:10803-12; Spivak et al., Clin Infect Dis 58:883-90). The data presented herein, demonstrates for the very first time the successful reactivation of HIV-1 latent proviruses by a novel dCas9-SAM technology. The salient finding of this study is the identification of highly effective and specific msgRNAs that induce sustained reactivation of the HIV-1 latent provirus and ultimately result in suicide death of HIV-1 infected cells (FIG. 13).

This HIV-1 latency-reversing system is innovative and exhibits several advantages over the current “shock and kill” strategy with latency-reversing chemicals or agents for the following reasons: (1) High Specificity: Target-specific msgRNAs were carefully designed through bioinformatics analysis and guided the novel dCas9-SAM to the HIV-1 promoter exclusively in HIV-infected cells. Additionally, a very short region was identified (around the enhancer adjacent to NF-κB binding sites) that is responsive to the dCas9-VPH reactivation. Finally, the suicide cell death is specifically dependent upon the production of HIV-1 toxic viral proteins. (2) High Efficiency: This dCas9-SAM technology delivers multiple exogenous transcriptional activators to the target site(s) and induces substantial increases in target cellular gene expression as compared to a single activator system (Konermann et al., 2015, Nature 517:583-8; Chavez et al., 2015, Nat Methods 12:326-8; Hilton et al., 2015, Nat Biotechnol 33:510-7). This study verified that the dCas9-SAM is more efficient than dCas9-VP64 alone in activating proviral production. This activation efficiency would not be affected by HIV-1 integration sites and endogenous epigenetic modification, which is a major obstacle for HIV-1 latency reactivation by the currently used chemicals or agents. (3) Sustained Reactivation Until All HIV-1 Latent Cells are Killed: The dCas9-VPH and msgRNAs are continuously expressed and/or inducibly controlled. Importantly, the dCas9 does not induce any indel mutation of the msgRNA target site that may prevent further binding of msgRNA to the targets (no self-limit). Therefore, the dCas9-SAM system is capable of retaining persistent levels of compulsory reactivation, leading to sustained and unlimited generation of viral proteins that will consequently kill the HIV-1 latent cells. This feature is very important for HIV-1 “shock and kill” strategy. (4) No/low Cytotoxicity to Neighboring HIV-Negative Cells: The dCas9 enzyme does not contain any nuclease activity, and thus would not induce any mutation or chromosome translocations/instabilities in the host cells. The dCas9-SAM system only affects HIV-infected cells, in contrast to currently developed chemical agents that exhibit non-specific effects on other non-infected cells. Even though the HIV-1 msgRNAs may have potential off-target sites (particularly with mismatch) in the host genome (extremely rare as shown in Cas9-sgRNA system), the possibility of the recruited dCas9-SAM complex to activate any potential pathogenic genes is extremely low because only 1.2% of genome encode functional genes and the number of pathogenic genes is extremely limited. (5) More msgRNAs Can Increase Proviral Reactivation Efficiency: As demonstrated in this work, multiple msgRNAs can be developed to increase reactivation efficiency. The synergistic action of LTR-L and LTR-O provides us a guide to develop an all-in-one viral or non-viral gene delivery system for further preclinical (animal) and clinical (patient) studies.

The ultimate goal of the latency-reversing strategy is to eliminate (kill) HIV-1-infected cells through toxic viral protein buildup and/or host immune clearance. In the culture system, the viral proteins play a major role in killing HIV-1 latent cells. In this study, the cell killing effect induced by the dCas9-VPH/msgRNAs system is completely dependent upon the generation of viral proteins as evidenced by (1) LTR potent reactivation induced cell death only in those cell lines that harbor the HIV-1 proviral genome (CHME5, 2D10, etc.) but not in TZM-bI cells that contain only the LTR-luciferase reporter; (2) Cell death depends upon the extent of LTR reactivation; (3) Establishing a stable cell line carrying the HIV-1 provirus and dCas9-VPH/msgRNAs was impossible due to viral protein-induced cell death; (4) A naïve and intact HIV-1 reporter virus can be largely propagated in the dCas9-VPH/msgRNAs LTR-L-expressing TZM-bI cells and ultimately kill the infected cells but replication/infection-deficient HIV-1 reporter viruses cannot. The identification of the specific viral proteins that induce suicide cell death during dCas9-SAM reactivation warrants further investigation.

By screening different sgRNAs or msgRNAs using dCas9-single activator or multiple activators, it was determined that the activation efficiency varied with different target sites as well as different dCas9 systems. This study focuses on the more effective dCas9-SAM system and demonstrated that LTR-L and O are the best msgRNAs to activate the HIV-1 LTR promoter, even though LTR-L and O exhibited various efficiencies of reactivation and apoptotic induction in different cell lines. However, it is likely that other combinations of multiple transcriptional activators (Chavez et al., 2015, Nat Methods 12:326-8; Hilton et al., 2015, Nat Biotechnol 33:510-7) may present different sgRNAs with best efficiency. It is also likely that LTR-L and O regulate various viral proteins in a different expression manner and through different molecular mechanisms.

Potential off-target effects remain a critical concern for the preclinical and clinical application of the CRISPR/Cas9 and its derived dCas9 system. Several promising strategies have been developed to mitigate any potential off-target responses, such as the sgRNA design bioinformatic optimization, transcriptome analysis, and functional screening after dCas9 treatment. For the parent Cas9-sgRNA system, more and more experimental data support that the genome editing is highly specific. Several reports using whole genome sequencing (WGS) at 15-100× coverage have demonstrated very rare instances of off-target effects while employing the Cas9/gRNA technology in vitro (Hu et al., 2014, PNAS 111:11461-6; Zuckermann et al., 2015, Nat Commun 6:7391; Smith et al., 2014, Cell Stem Cell 14:12-3; Veres et al., 2014, Cell Stem Cell 14:27-30; Yang et al, 2014, Nat Commun 5:5507). Newly developed unbiased profiling techniques further validate the high specificity of this Cas9-sgRNA system (Ran et al., 2015, Nature 520:186-91; Tsai et al., 2015, Nat Biotechnol 33:187-97; Frock et al., 2015, Nat Biotechnol 33:179-86). In vivo off-target is expected to be much lower due to epigenetic protection. In addition, the off-target frequency in essential gene/genome will be very rare because exons comprise only 1.2% of the entire genome. In the case of dCas9 system, the frequency of off-target binding to essential (functional) exons would also be very low. RNA-seq analysis confirmed the specificity of this dCas9-SAM technology (Konermann et al., 2015, Nature 517:583-8). In the present study, the exogenous viral DNA was analyzed against the host genome for best score of efficiency and specificity. In TZM-bI cells without viral protein production, the dCas9-VPH/msgRNAs induced potent reactivation of LTR-eLuc reporter, but did not influence the cell growth/proliferation, supporting the absence of off-target effects by the dCas9-VPH/LTR-msgRNA system. Nevertheless, further analysis by RNA-sequencing, RT-PCR array or microarray is warranted.

The HIV-1 genome contains almost identical 5′- and 3′-end LTRs. The 5′-LTR normally functions as an RNA polymerase II promoter but the 3′ LTR acts in transcription termination/polyadenylation and is not normally functional as a promoter due to transcriptional interference (Klaver and Berkhout, 1994, J Virol 68:3830-40). Such transcriptional suppression is attributed to the competition of endogenous transcriptional factors between 5′- and 3′-LTR promoter (Klaver and Berkhout, 1994, J Virol 68:3830-40; Boerkoel and Kung, 1992, J Virol 66:4814-23). In this study, the msgRNAs designed for 5′-LTR may also affect the 3′-LTR promoter activity because the recruitment of SAM to target specific region is independent from the endogenous transcriptional factors. Additional activation of the integrated HIV-1 provirus via 3′-LTR promoter by dCas9-SAM system might be another advantage of this novel approach over the currently-used chemical agents. This proof of concept is worthwhile of further investigation using an indicator gene downstream of the 3′ LTR (Cullen et al., 1984, Nature 307:241-5).

The data presented herein demonstrates that the latent HIV-1 provirus can be reactivated dramatically by the engineered dCas9-SAM guided by msgRNAs specifically targeting the enhancer of the HIV-1 LTR promoter (FIG. 13). This study presents a novel, early-stage preclinical approach that will result in the development of an additional “shock and kill” therapeutic strategy to eliminate the HIV-1 latent reservoir in a clinical setting. One major factor that is slowing the dCas9-SAM system's path to clinical trials is the low gene delivery efficiency to HIV-1 latent cells. Improvement in the viral and non-viral gene delivery in the field of gene therapeutics will boost the clinical application of the CRISPR/Cas9 technology.

TABLE 3
Sequences and locations
for msgRNA targeting sites

| Target | | | |
|---|---|---|---|
| Name | To TSS | Direction | Sequences (5′ to 3′) |
| LTR-C | -375 to -377 | T357: Sense | caccGATTGGCAGAACTACACACC |
| | | | (SEQ ID NO: 109) |
| | | T358: Antisense | aaacGGTGTGTAGTTCTGCCAATC |
| | | | (SEQ ID NO: 110) |
| LTR-D | -55 to -75 | T359: Sense | caccGCGTGGCCTGGGCGGGACTG |
| | | | (SEQ ID NO: 111) |
| | | T360: Antisense | aaacCAGTCCCGCCCAGGCCACGC |
| | | | (SEQ ID NO: 112) |
| LTR-E | -390 to -412 | T361: Sense | caccGATCTGTGGATCTACCACACACA |
| | | | (SEQ ID NO: 113) |
| | | T362: Antisense | aaacTGTGTGTGGTAGATCCACAGATC |
| | | | (SEQ ID NO: 114) |
| LTR-F | -18 to -40 | T363: Sense | caccGCTGCTTATATGCAGCATCTGAG |
| | | | (SEQ ID NO: 115) |
| | | T364: Antisense | aaacCTCAGATGCTGCATATAAGCAGC |
| | | | (SEQ ID NO: 116) |
| LTR-G | -393 to -413 | T530: Sense | caccGTGTGGTAGATCCACAGATCA |
| | | | (SEQ ID NO: 117) |
| | | T531: Antisense | aaacTGATCTGTGGATCTACCACAC |
| | | | (SEQ ID NO: 118) |
| LTR-H | -375 to -395 | T532: Sense | caccGCAGGGAAGTAGCCTTGTGTG |
| | | | (SEQ ID NO: 119) |
| | | T533: Antisense | aaacCACACAAGGCTACTTCCCTGC |
| | | | (SEQ ID NO: 120) |
| LTR-I | -327 to -427 | T534: Sense | caccGATCAGATATCCACTGACCTT |
| | | | (SEQ ID NO: 121) |
| | | T535: Antisense | aaacAAGGTCAGTGGATATCTGATC |
| | | | (SEQ ID NO: 122) |
| LTR-J | -194 to -214 | T536: Sense | caccGCACACTAATACTTCTCCCTC |
| | | | (SEQ ID NO: 123) |
| | | T537: Antisense | aaacGAGGGAGAAGTATTAGTGTGC |
| | | | (SEQ ID NO: 124) |
| LTR-K | -163 to -183 | T538: Sense | caccGCCTCCTAGCATTTCGTCACA |
| | | | (SEQ ID NO: 125) |
| | | T539: Antisense | aaacTGTGACGAAATGCTAGGAGGC |
| | | | (SEQ ID NO: 126) |
| LTR-L | -145 to -165 | T540: Sense | caccGCATGGCCCGAGAGCTGCATC |
| | | | (SEQ ID NO: 127) |
| | | T541: Antisense | aaacGATGCAGCTCTCGGGCCATGC |
| | | | (SEQ ID NO: 128) |
| LTR-M | -122 to -142 | T542: Sense | caccGCAGCAGTCTTTGTAGTACTC |
| | | | (SEQ ID NO: 129) |
| | | T543: Antisense | aaacGAGTACTACAAAGACTGCTGC |
| | | | (SEQ ID NO: 130) |
| LTR-N | -106 to -126 | T544: Sense | caccGCTGACATCGAGCTTTCTACA |
| | | | (SEQ ID NO: 131) |
| | | T545: Antisense | aaacTGTAGAAAGCTCGATGTCAGC |
| | | | (SEQ ID NO: 132) |
| LTR-0 | -92 to -112 | T546: Sense | caccGTCTACAAGGGACTTTCCGCT |
| | | | (SEQ ID NO: 133) |
| | | T547: Antisense | aaacAGCGGAAAGTCCCTTGTAGAC |
| | | | (SEQ ID NO: 134) |
| LTR-P | -81 to -102 | T548: Sense | caccGCTTTCCGCTGGGGACTTTCC |
| | | | (SEQ ID NO: 135) |
| | | T549: Antisense | aaacGGAAAGTCCCCAGCGGAAAGC |
| | | | (SEQ ID NO: 136) |
| LTR-Ra | 61 to 81 | T422: Sense | caccGCTTTATTGAGGCTTAAGCAG |
| | | | (SEQ ID NO: 137) |
| | | T423: Antisense | aaacCTGCTTAAGCCTCAATAAAGC |
| | | | (SEQ ID NO: 138) |
| LTR-U5a | 109 to 128 | T424: Sense | caccGCCCGTCTGTTGTGTGACTC |
| | | | (SEQ ID NO: 139) |
| | | T425: Antisense | aaacGAGTCACACAACAGACGGGC |
| | | | (SEQ ID NO: 140) |

TABLE 4
Primers used for sequencing and PCR
Sequencing

| | |
|---|---|
| hU6-sequence | ATGGACTATCATATGCTTACCG |
| | (SEQ ID NO: 141) |
| Flap-Seq/5′/F | CAGTGCAGGGGAAAGAATAGTAGAC |
| | (SEQ ID NO: 142) |
| SCV-Rev | CAGCGGGGCTGCTAAAGCGCATGC |
| | (SEQ ID NO: 143) |

PCR primer

| | |
|---|---|
| HIV-EcoEnv-F1 | TGGGATATTGATGATCTGTAGTGCTATGGCGC |
| | GTTCAACGCTCT (SEQ ID NO: 144) |
| HIV-EcoEnv-R1 | AGAGCGTTGAACGCGCCATAGCACTACAGATC |
| | ATCAATATCCCA (SEQ ID NO: 145) |
| HIV-EcoEnv-F2 | GCCTATAGAGTACGAGCCATAGGTGGGAATAG |
| | GAGCTTTGTTCC (SEQ ID NO: 146) |
| HIV-EcoEnv-R2 | GGAACAAAGCTCCTATTCCCACCTATGGCTCG |
| | TACTCTATAGGC (SEQ ID NO: 147) |
| HIV-Env-opti- | GCTTGGAAAGGATTTTGCTATAAGCTAGCCAC |
| luc-F | CATGGAAGATGCCAAGAACATCAAGA |
| | (SEQ ID NO: 148) |
| HIV-Env-opti- | TCTTGATGTTCTTGGCATCTTCCATGGTGGCT |
| luc-R | AGCTTATAGCAAAATCCTTTCCAAGC |
| | (SEQ ID NO: 149) |
| Opti-luc-P2A-F | GCCAAGAAGGGCGGCAAGCCTAGGCCTGCCAC |
| | GAACTTCTCTCTGTTAAAG |
| | (SEQ ID NO: 150) |
| Opti-luc-P2A-R | CTTTAACAGAGAGAAGTTCGTGGCAGGCCTAG |
| | GCTTGCCGCCCTTCTTGGC |
| | (SEQ ID NO: 151) |
| Opti-luc-P2A-R | CTTTAACAGAGAGAAGTTCGTGGCAGGCCTAG |
| | GCTTGCCGCCCTTCTTGGC |
| | (SEQ ID NO: 152) |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for reactivation of a retrovirus in vitro or in vivo comprising: an isolated nucleic acid encoding a guide nucleic acid, wherein the guide nucleic acid comprises a targeting nucleotide sequence directed to one or more target sequences in the retroviral genome; an isolated nucleic acid encoding a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

2. The composition of claim 1, wherein the CRISPR/Cas fusion protein comprises catalytically deficient Cas protein (dCas), orthologs, homologs, mutants variants or fragments thereof, fused with one or more transcriptional activators.

3. The composition of claim 1, wherein the one or more target sequences comprises one or more sequences within a human immunodeficiency virus (HIV) LTR.

4. The composition of claim 1, wherein the RNA binding protein, fragments, mutants, derivatives or variants thereof, comprises a bacteriophage coat protein.

5. The composition of claim 1, wherein the one or more transcriptional activators comprise VP64, p65, HSF1, p65AD, Rta, Sp1, Vax, GATA4, fragments, mutants, or any combinations thereof.

6. The composition of claim 1, wherein the one or more target sequences comprise a nucleic acid sequence having at least about 75% sequence similarity to any one or more sequences comprising SEQ ID NOS: 1-152 or combinations thereof.

7. The composition of claim 1, wherein the one or more target sequences comprise one or more nucleic acid sequences comprising SEQ ID NOS: 1-152 or combinations thereof.

8. An isolated nucleic acid encoding: a guide nucleic acid, wherein the guide nucleic acid comprises a targeting nucleotide sequence directed to one or more target sequences in the retroviral genome; a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

9. The isolated nucleic acid of claim 8, wherein the CRISPR/Cas fusion protein comprises catalytically deficient Cas protein (dCas), orthologs, homologs, mutants variants or fragments thereof, fused with one or more transcriptional activators.

10. The isolated nucleic acid of claim 8, wherein the one or more target sequences comprises one or more sequences within a human immunodeficiency virus (HIV) LTR.

11. The isolated nucleic acid of claim 8, wherein the RNA binding protein, fragments, mutants, derivatives or variants thereof, comprises a bacteriophage coat protein.

12. The isolated nucleic acid of claim 8, wherein the one or more transcriptional activators comprise VP64, p65, HSF1, p65AD, Rta, Sp1, Vax, GATA4, fragments, mutants, or any combinations thereof.

13. The isolated nucleic acid of claim 8, wherein the one or more target sequences comprise a nucleic acid sequence having at least about 75% sequence similarity to any one or more sequences comprising SEQ ID NOS: 1-152 or combinations thereof.

14. The isolated nucleic acid of claim 8, wherein the one or more target sequences comprise one or more nucleic acid sequences comprising SEQ ID NOS: 1-152 or combinations thereof.

15. An isolated nucleic acid molecule encoding at least one guide nucleic acid molecule (gRNA), an isolated nucleic acid encoding a Clustered Regularly Interspaced Short Palindromic Repeat (CRISPR)-associated endonuclease/Cas (CRISPR/Cas) fusion protein or functional fragment or derivative thereof, and an isolated nucleic acid encoding an RNA binding protein, functional fragments, mutants, derivatives or variants thereof.

16. The isolated nucleic acid of claim 15, wherein the CRISPR/Cas fusion protein comprises catalytically deficient Cas protein (dCas), orthologs, homologs, mutants variants or fragments thereof, fused with one or more transcriptional activators.

17. The isolated nucleic acid of claim 15, wherein the RNA binding protein, fragments, mutants, derivatives or variants thereof, comprises a bacteriophage coat protein.

18. The isolated nucleic acid of claim 16, wherein the one or more transcriptional activators comprise VP64, p65, HSF1, p65AD, Rta, Sp1, Vax, GATA4, fragments, mutants, or any combinations thereof.

19. The isolated nucleic acid of claim 15, wherein the one or more target sequences comprise a nucleic acid sequence having at least about 75% sequence similarity to any one or more sequences comprising SEQ ID NOS: 1-152 or combinations thereof.

20. The isolated nucleic acid of claim 15, wherein the one or more target sequences comprise one or more nucleic acid sequences comprising SEQ ID NOS: 1-152 or combinations thereof.

21. A composition for reactivation of HIV in a latently infected cell comprising:
a) one selected from the group consisting of an isolated guide nucleic acid and an isolated nucleic acid encoding a guide nucleic acid, wherein the guide nucleic acid comprises a targeting nucleotide sequence directed to a target sequence in the HIV genome;
b) one selected from the group consisting of a catalytically deficient Cas9 (dCas9) fusion protein, and an isolated nucleic acid encoding a dCas9 fusion protein, wherein the dCas9 fusion protein comprises dCas9 and one or more transcriptional activators; and
c) one selected from the group consisting of a MS2 fusion protein, and an isolated nucleic acid encoding a MS2 fusion protein, wherein the MS2 fusion protein comprises MS2 bacteriophage coat protein and one or more transcription activators.

22. The composition of claim 21, wherein the target sequence comprises a sequence within HIV LTR.

23. The composition of claim 21, wherein the target sequence comprises a sequence within the enhancer or core promoter region of HIV LTR.

24. The composition of claim 21, wherein the guide nucleic acid is RNA.

25. The composition of claim 21, wherein the guide nucleic acid comprises crRNA and tracrRNA.

26. The composition of claim 21, wherein the one or more transcription activators are selected from the group consisting of VP64, p65, HSF1, p65AD, Rta, Sp1, Vax, GATA4, and any combination thereof.

27. The composition of claim 21, wherein the target sequence comprises one or more nucleic acid sequences comprising SEQ ID NOS: 1-152 or combinations thereof.

28. The composition of claim 21, wherein the target sequence is selected from the group consisting of SEQ ID NO: 94 (LTR-J), SEQ ID NO: 96 (LTR-L), SEQ ID NO: 98 (LTR-N) and SEQ ID NO: 99 (LTR-O).

29. The composition of claim 21, wherein the guide nucleic acid comprises a hairpin aptamer capable of binding to MS2.

30. The composition of claim 21, wherein the composition comprises one or more isolated nucleic acids, where the one or more isolated nucleic acids encode multiple guide nucleic acids, wherein each guide nucleic acid comprises a targeting nucleotide sequence directed to a different target sequence in the HIV genome.

31. A pharmaceutical composition comprising
a) one selected from the group consisting of an isolated guide nucleic acid and an isolated nucleic acid encoding a guide nucleic acid, wherein the guide nucleic acid comprises a targeting nucleotide sequence directed to a target sequence in the HIV genome;
b) one selected from the group consisting of a catalytically deficient Cas9 (dCas9) fusion protein, and an isolated nucleic acid encoding a dCas9 fusion protein, wherein the dCas9 fusion protein comprises dCas9 and one or more transcription activators; and
c) one selected from the group consisting of a MS2 fusion protein, and an isolated nucleic acid encoding a MS2 fusion protein, wherein the MS2 fusion protein comprises MS2 bacteriophage coat protein and one or more transcription activators.

32. An expression vector encoding at least one of: a guide nucleic acid, a dCas9 fusion protein, and a MS2 fusion protein, wherein
the guide nucleic acid comprises a targeting nucleotide sequence directed to a target sequence in the HIV genome;
the dCas9 fusion protein comprises dCas9 and one or more transcription activators; and
the MS2 fusion protein comprises MS2 bacteriophage coat protein and one or more transcription activators.

33. A host cell comprising the expression vector comprising:
(i) an isolated nucleic acid encoding a guide nucleic acid, wherein the guide nucleic acid comprises a targeting nucleotide sequence directed to one or more target sequences in the retroviral genome; a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators; or,
(ii) an isolated nucleic acid molecule encoding at least one guide nucleic acid molecule (gRNA), an isolated nucleic acid encoding a Clustered Regularly Interspaced Short Palindromic Repeat (CRISPR)-associated endonuclease/Cas (CRISPR/Cas) fusion protein or functional fragment or derivative thereof, and an isolated nucleic acid encoding an RNA binding protein, functional fragments, mutants, derivatives or variants thereof; or,
(iii) (a) an isolated guide nucleic acid and an isolated nucleic acid encoding a guide nucleic acid, wherein the guide nucleic acid comprises a targeting nucleotide sequence directed to a target sequence in the HIV genome;
(b) a catalytically deficient Cas9 (dCas9) fusion protein, and an isolated nucleic acid encoding a dCas9 fusion protein, wherein the dCas9 fusion protein comprises dCas9 and one or more transcriptional activators; and
(c) a MS2 fusion protein, and an isolated nucleic acid encoding a MS2 fusion protein, wherein the MS2 fusion protein comprises MS2 bacteriophage coat protein and one or more transcription activators; or,
(iv) at least one of: a guide nucleic acid, a dCas9 fusion protein, and a MS2 fusion protein, wherein the guide nucleic acid comprises a targeting nucleotide sequence directed to a target sequence in the HIV genome;
the dCas9 fusion protein comprises dCas9 and one or more transcription activators; and
the MS2 fusion protein comprises MS2 bacteriophage coat protein and one or more transcription activators.

34. A method for reactivation of HIV in a latently infected cell in a subject, comprising contacting a cell of the subject with a therapeutically effective amount of a composition comprising
a) one selected from the group consisting of an isolated guide nucleic acid and an isolated nucleic acid encoding a guide nucleic acid, where the guide nucleic acid comprises a targeting nucleotide sequence directed to a target sequence in the HIV genome;
b) one selected from the group consisting of a catalytically deficient Cas9 (dCas9) fusion protein, and an isolated nucleic acid encoding a dCas9 fusion protein, wherein the dCas9 fusion protein comprises dCas9 and one or more transcription activators; and
c) one selected from the group consisting of a MS2 fusion protein, and an isolated nucleic acid encoding a MS2 fusion protein, wherein the MS2 fusion protein comprises MS2 bacteriophage coat protein and one or more transcription activators.

35. The method of claim 38, wherein the subject is diagnosed with having HIV.

36. The method of claim 38, wherein the subject has a latent HIV infection.


SEQUENCE LISTING (152 sequences)

SEQ ID NO: 1 - DNA, 634 bp, Human immunodeficiency virus 1
tggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 30 bp, Artificial Sequence
  Description of Artificial Sequence Synthetic oligonucleotide
cgaagacaagatatccttgatctgtggatc

SEQ ID NO: 3 - DNA, 30 bp, Artificial Sequence
  Description of Artificial Sequence Synthetic oligonucleotide
tgatctgtggatctaccacacacaaggcta

SEQ ID NO: 4 - DNA, 30 bp, Artificial Sequence
  Description of Artificial Sequence Synthetic oligonucleotide
cacacacaaggctacttccctgattggcag

SEQ ID NO: 5 - DNA, 30 bp, Artificial Sequence
  Description of Artificial Sequence Synthetic oligonucleotide
ccctgattggcagaactacacaccagggcc

SEQ ID NO: 6 - DNA, 30 bp, Artificial Sequence
  Description of Artificial Sequence Synthetic oligonucleotide
cctgattggcagaactacacaccagggcca

SEQ ID NO: 7 - DNA, 30 bp, Artificial Sequence
  Description of Artificial Sequence Synthetic oligonucleotide
ttggcagaactacacaccagggccagggat

SEQ ID NO: 8 - DNA, 30 bp, Artificial Sequence
  Description of Artificial Sequence Synthetic oligonucleotide
tggcagaactacacaccagggccagggatc

SEQ ID NO: 9 - DNA, 30 bp, Artificial Sequence
  Description of Artificial Sequence Synthetic oligonucleotide
agggatcagatatccactgacctttggatg

SEQ ID NO: 10 - DNA, 30 bp, Artificial Sequence
  Description of Artificial Sequence Synthetic oligonucleotide
atcagatatccactgacctttggatggtgc

SEQ ID NO: 11 - DNA, 30 bp, Artificial Sequence
  Description of Artificial Sequence Synthetic oligonucleotide
agctagtaccagttgagcaagagaaggtag

SEQ ID NO: 12 - DNA, 30 bp, Artificial Sequence
  Description of Artificial Sequence Synthetic oligonucleotide
agagaaggtagaagaagccaatgaaggaga

SEQ ID NO: 13 - DNA, 30 bp, Artificial Sequence
  Description of Artificial Sequence Synthetic oligonucleotide
cttgttacaccctgtgagcctgcatgggat

SEQ ID NO: 14 - DNA, 30 bp, Artificial Sequence
  Description of Artificial Sequence Synthetic oligonucleotide
ttgttacaccctgtgagcctgcatgggatg

SEQ ID NO: 15 - DNA, 30 bp, Artificial Sequence
  Description of Artificial Sequence Synthetic oligonucleotide
tacaccctgtgagcctgcatgggatggatg

SEQ ID NO: 16 - DNA, 30 bp, Artificial Sequence
  Description of Artificial Sequence Synthetic oligonucleotide
tgagcctgcatgggatggatgacccggaga

SEQ ID NO: 17 - DNA, 30 bp, Artificial Sequence
  Description of Artificial Sequence Synthetic oligonucleotide
gacccggagagagaagtattagagtggagg

SEQ ID NO: 18 - DNA, 30 bp, Artificial Sequence
  Description of Artificial Sequence Synthetic oligonucleotide
ccggagagagaagtattagagtggaggttt

SEQ ID NO: 19 - DNA, 30 bp, Artificial Sequence
  Description of Artificial Sequence Synthetic oligonucleotide
acagccgcctagcatttcatcacatggccc

SEQ ID NO: 20 - DNA, 30 bp, Artificial Sequence
  Description of Artificial Sequence Synthetic oligonucleotide
atcacatggcccgagagctgcatccggagt

SEQ ID NO: 21 - DNA, 30 bp, Artificial Sequence
  Description of Artificial Sequence Synthetic oligonucleotide
actgctgacatcgagcttgctacaagggac

SEQ ID NO: 22 - DNA, 30 bp, Artificial Sequence
  Description of Artificial Sequence Synthetic oligonucleotide
ctgctgacatcgagcttgctacaagggact

SEQ ID NO: 23 - DNA, 30 bp, Artificial Sequence
  Description of Artificial Sequence Synthetic oligonucleotide
agcttgctacaagggactttccgctgggga

SEQ ID NO: 24 - DNA, 30 bp, Artificial Sequence
  Description of Artificial Sequence Synthetic oligonucleotide
gcttgctacaagggactttccgctggggac

SEQ ID NO: 25 - DNA, 30 bp, Artificial Sequence
  Description of Artificial Sequence Synthetic oligonucleotide
cttgctacaagggactttccgctggggact

SEQ ID NO: 26 - DNA, 30 bp, Artificial Sequence
  Description of Artificial Sequence Synthetic oligonucleotide
gggactttccgctggggactttccagggag

SEQ ID NO: 27 - DNA, 30 bp, Artificial Sequence
  Description of Artificial Sequence Synthetic oligonucleotide
ggactttccgctggggactttccagggagg

SEQ ID NO: 28 - DNA, 30 bp, Artificial Sequence
  Description of Artificial Sequence Synthetic oligonucleotide
ctttccgctggggactttccagggaggcgt

SEQ ID NO: 29 - DNA, 30 bp, Artificial Sequence
  Description of Artificial Sequence Synthetic oligonucleotide
cgctggggactttccagggaggcgtggcct

SEQ ID NO: 30 - DNA, 30 bp, Artificial Sequence
  Description of Artificial Sequence Synthetic oligonucleotide
gggactttccagggaggcgtggcctgggcg

SEQ ID NO: 31 - DNA, 30 bp, Artificial Sequence
  Description of Artificial Sequence Synthetic oligonucleotide
ggactttccagggaggcgtggcctgggcgg

SEQ ID NO: 32 - DNA, 30 bp, Artificial Sequence
  Description of Artificial Sequence Synthetic oligonucleotide
ctttccagggaggcgtggcctgggcgggac

SEQ ID NO: 33 - DNA, 30 bp, Artificial Sequence
  Description of Artificial Sequence Synthetic oligonucleotide
tttccagggaggcgtggcctgggcgggact

SEQ ID NO: 34 - DNA, 30 bp, Artificial Sequence
  Description of Artificial Sequence Synthetic oligonucleotide
agggaggcgtggcctgggcgggactgggga

SEQ ID NO: 35 - DNA, 30 bp, Artificial Sequence
  Description of Artificial Sequence Synthetic oligonucleotide
gggaggcgtggcctgggcgggactggggag

SEQ ID NO: 36 - DNA, 30 bp, Artificial Sequence
  Description of Artificial Sequence Synthetic oligonucleotide
ggaggcgtggcctgggcgggactggggagt

SEQ ID NO: 37 - DNA, 30 bp, Artificial Sequence
  Description of Artificial Sequence Synthetic oligonucleotide
cgtggcctgggcgggactggggagtggcga

SEQ ID NO: 38 - DNA, 30 bp, Artificial Sequence
  Description of Artificial Sequence Synthetic oligonucleotide
ctttttgcttgtactgggtctctctggtta

SEQ ID NO: 39 - DNA, 30 bp, Artificial Sequence
  Description of Artificial Sequence Synthetic oligonucleotide
ctctggttagaccagatctgagcctgggag

SEQ ID NO: 40 - DNA, 30 bp, Artificial Sequence
  Description of Artificial Sequence Synthetic oligonucleotide
tctggttagaccagatctgagcctgggagc

SEQ ID NO: 41 - DNA, 30 bp, Artificial Sequence
  Description of Artificial Sequence Synthetic oligonucleotide
ccagatctgagcctgggagctctctggcta

SEQ ID NO: 42 - DNA, 30 bp, Artificial Sequence
  Description of Artificial Sequence Synthetic oligonucleotide
agcctgggagctctctggctaactagggaa

SEQ ID NO: 43 - DNA, 30 bp, Artificial Sequence
  Description of Artificial Sequence Synthetic oligonucleotide
gcctgggagctctctggctaactagggaac

SEQ ID NO: 44 - DNA, 30 bp, Artificial Sequence
  Description of Artificial Sequence Synthetic oligonucleotide
gtgtgcccgtctgttgtgtgactctggtaa

SEQ ID NO: 45 - DNA, 30 bp, Artificial Sequence
  Description of Artificial Sequence Synthetic oligonucleotide
gatcaaggatatcttgtcttcgttgggagt

SEQ ID NO: 46 - DNA, 30 bp, Artificial Sequence
  Description of Artificial Sequence Synthetic oligonucleotide
agatcaaggatatcttgtcttcgttgggag

SEQ ID NO: 47 - DNA, 30 bp, Artificial Sequence
  Description of Artificial Sequence Synthetic oligonucleotide
tgtgtgtggtagatccacagatcaaggata

SEQ ID NO: 48 - DNA, 30 bp, Artificial Sequence
  Description of Artificial Sequence Synthetic oligonucleotide
caatcagggaagtagccttgtgtgtggtag

SEQ ID NO: 49 - DNA, 30 bp, Artificial Sequence
  Description of Artificial Sequence Synthetic oligonucleotide
cctggtgtgtagttctgccaatcagggaag

SEQ ID NO: 50 - DNA, 30 bp, Artificial Sequence
  Description of Artificial Sequence Synthetic oligonucleotide
ccctggtgtgtagttctgccaatcagggaa

SEQ ID NO: 51 - DNA, 30 bp, Artificial Sequence
  Description of Artificial Sequence Synthetic oligonucleotide
cagtggatatctgatccctggccctggtgt

SEQ ID NO: 52 - DNA, 30 bp, Artificial Sequence
  Description of Artificial Sequence Synthetic oligonucleotide
aaaggtcagtggatatctgatccctggccc

SEQ ID NO: 53 - DNA, 30 bp, Artificial Sequence
  Description of Artificial Sequence Synthetic oligonucleotide
cttgtagcaccatccaaaggtcagtggata

SEQ ID NO: 54 - DNA, 30 bp, Artificial Sequence
  Description of Artificial Sequence Synthetic oligonucleotide
gtactagcttgtagcaccatccaaaggtca

SEQ ID NO: 55 - DNA, 30 bp, Artificial Sequence
  Description of Artificial Sequence Synthetic oligonucleotide
ttcttctaccttctcttgctcaactggtac

SEQ ID NO: 56 - DNA, 30 bp, Artificial Sequence
  Description of Artificial Sequence Synthetic oligonucleotide
acaagcgggtgttctctccttcattggctt

SEQ ID NO: 57 - DNA, 30 bp, Artificial Sequence
  Description of Artificial Sequence Synthetic oligonucleotide
gcaggctcacagggtgtaacaagcgggtgt

SEQ ID NO: 58 - DNA, 30 bp, Artificial Sequence
  Description of Artificial Sequence Synthetic oligonucleotide
tgcaggctcacagggtgtaacaagcgggtg

SEQ ID NO: 59 - DNA, 30 bp, Artificial Sequence
  Description of Artificial Sequence Synthetic oligonucleotide
tcatccatcccatgcaggctcacagggtgt

SEQ ID NO: 60 - DNA, 30 bp, Artificial Sequence
  Description of Artificial Sequence Synthetic oligonucleotide
gtcatccatcccatgcaggctcacagggtg

SEQ ID NO: 61 - DNA, 30 bp, Artificial Sequence
  Description of Artificial Sequence Synthetic oligonucleotide
ctctccgggtcatccatcccatgcaggctc

SEQ ID NO: 62 - DNA, 30 bp, Artificial Sequence
  Description of Artificial Sequence Synthetic oligonucleotide
ctccactctaatacttctctctccgggtca

SEQ ID NO: 63 - DNA, 30 bp, Artificial Sequence
  Description of Artificial Sequence Synthetic oligonucleotide
cctccactctaatacttctctctccgggtc

SEQ ID NO: 64 - DNA, 30 bp, Artificial Sequence
  Description of Artificial Sequence Synthetic oligonucleotide
cgggccatgtgatgaaatgctaggcggctg

SEQ ID NO: 65 - DNA, 30 bp, Artificial Sequence
  Description of Artificial Sequence Synthetic oligonucleotide
tctcgggccatgtgatgaaatgctaggcgg

SEQ ID NO: 66 - DNA, 30 bp, Artificial Sequence
  Description of Artificial Sequence Synthetic oligonucleotide
tgaagtactccggatgcagctctcgggcca

SEQ ID NO: 67 - DNA, 30 bp, Artificial Sequence
  Description of Artificial Sequence Synthetic oligonucleotide
ttgaagtactccggatgcagctctcgggcc

SEQ ID NO: 68 - DNA, 30 bp, Artificial Sequence
  Description of Artificial Sequence Synthetic oligonucleotide
atgtcagcagttcttgaagtactccggatg

SEQ ID NO: 69 - DNA, 30 bp, Artificial Sequence
  Description of Artificial Sequence Synthetic oligonucleotide
cacgcctccctggaaagtccccagcggaaa

SEQ ID NO: 70 - DNA, 30 bp, Artificial Sequence
  Description of Artificial Sequence Synthetic oligonucleotide
agtcccgcccaggccacgcctccctggaaa

SEQ ID NO: 71 - DNA, 30 bp, Artificial Sequence
  Description of Artificial Sequence Synthetic oligonucleotide
gctcgccactccccagtcccgcccaggcca

SEQ ID NO: 72 - DNA, 30 bp, Artificial Sequence
  Description of Artificial Sequence Synthetic oligonucleotide
cagctgcttatatgcagcatctgagggctc

SEQ ID NO: 73 - DNA, 30 bp, Artificial Sequence
  Description of Artificial Sequence Synthetic oligonucleotide
gcagctgcttatatgcagcatctgagggct

SEQ ID NO: 74 - DNA, 30 bp, Artificial Sequence
  Description of Artificial Sequence Synthetic oligonucleotide
ccagagagctcccaggctcagatctggtct

SEQ ID NO: 75 - DNA, 30 bp, Artificial Sequence
  Description of Artificial Sequence Synthetic oligonucleotide
tccctagttagccagagagctcccaggctc

SEQ ID NO: 76 - DNA, 30 bp, Artificial Sequence
  Description of Artificial Sequence Synthetic oligonucleotide
aagctttattgaggcttaagcagtgggttc

SEQ ID NO: 77 - DNA, 30 bp, Artificial Sequence
  Description of Artificial Sequence Synthetic oligonucleotide
caagctttattgaggcttaagcagtgggtt

SEQ ID NO: 78 - DNA, 30 bp, Artificial Sequence
  Description of Artificial Sequence Synthetic oligonucleotide
aagcactcaaggcaagctttattgaggctt

SEQ ID NO: 79 - DNA, 30 bp, Artificial Sequence
  Description of Artificial Sequence Synthetic oligonucleotide
ggcacacactacttgaagcactcaaggcaa

SEQ ID NO: 80 - DNA, 30 bp, Artificial Sequence
  Description of Artificial Sequence Synthetic oligonucleotide
agttaccagagtcacacaacagacgggcac

SEQ ID NO: 81 - DNA, 30 bp, Artificial Sequence
  Description of Artificial Sequence Synthetic oligonucleotide
tagttaccagagtcacacaacagacgggca

SEQ ID NO: 82 - DNA, 30 bp, Artificial Sequence
  Description of Artificial Sequence Synthetic oligonucleotide
tccacactgactaaaagggtctgagggatc

SEQ ID NO: 83 - DNA, 30 bp, Artificial Sequence
  Description of Artificial Sequence Synthetic oligonucleotide
ttccacactgactaaaagggtctgagggat

SEQ ID NO: 84 - DNA, 18 bp, Artificial Sequence
  Description of Artificial Sequence Synthetic oligonucleotide
gggtcttcgagaagacct

SEQ ID NO: 85 - DNA, 21 bp, Artificial Sequence
  Description of Artificial Sequence Synthetic oligonucleotide
gatcagatatccactgacctt

SEQ ID NO: 86 - DNA, 21 bp, Artificial Sequence
  Description of Artificial Sequence Synthetic oligonucleotide
gcagcagttcttgaagtactc

SEQ ID NO: 87 - DNA, 20 bp, Artificial Sequence
  Description of Artificial Sequence Synthetic oligonucleotide
gattggcagaactacacacc

SEQ ID NO: 88 - DNA, 20 bp, Artificial Sequence
  Description of Artificial Sequence Synthetic oligonucleotide
gcgtggcctgggcgggactg

SEQ ID NO: 89 - DNA, 23 bp, Artificial Sequence
  Description of Artificial Sequence Synthetic oligonucleotide
gatctgtggatctaccacacaca

SEQ ID NO: 90 - DNA, 23 bp, Artificial Sequence
  Description of Artificial Sequence Synthetic oligonucleotide
gctgcttatatgcagcatctgag

SEQ ID NO: 91 - DNA, 21 bp, Artificial Sequence
  Description of Artificial Sequence Synthetic oligonucleotide
gtgtggtagatccacagatca

SEQ ID NO: 92 - DNA, 21 bp, Artificial Sequence
  Description of Artificial Sequence Synthetic oligonucleotide
gcagggaagtagccttgtgtg

SEQ ID NO: 93 - DNA, 21 bp, Artificial Sequence
  Description of Artificial Sequence Synthetic oligonucleotide
gatcagatatccactgacctt

SEQ ID NO: 94 - DNA, 21 bp, Artificial Sequence
  Description of Artificial Sequence Synthetic oligonucleotide
gcacactaatacttctccctc

SEQ ID NO: 95 - DNA, 21 bp, Artificial Sequence
  Description of Artificial Sequence Synthetic oligonucleotide
gcctcctagcatttcgtcaca

SEQ ID NO: 96 - DNA, 21 bp, Artificial Sequence
  Description of Artificial Sequence Synthetic oligonucleotide
gcatggcccgagagctgcatc

SEQ ID NO: 97 - DNA, 21 bp, Artificial Sequence
  Description of Artificial Sequence Synthetic oligonucleotide
gcagcagtctttgtagtactc

SEQ ID NO: 98 - DNA, 21 bp, Artificial Sequence
  Description of Artificial Sequence Synthetic oligonucleotide
gctgacatcgagctttctaca

SEQ ID NO: 99 - DNA, 21 bp, Artificial Sequence
  Description of Artificial Sequence Synthetic oligonucleotide
gtctacaagggactttccgct

SEQ ID NO: 100 - DNA, 21 bp, Artificial Sequence
  Description of Artificial Sequence Synthetic oligonucleotide
gctttccgctggggactttcc

SEQ ID NO: 101 - DNA, 21 bp, Artificial Sequence
  Description of Artificial Sequence Synthetic oligonucleotide
gcctccctggaaagtccccag

SEQ ID NO: 102 - DNA, 21 bp, Artificial Sequence
  Description of Artificial Sequence Synthetic oligonucleotide
gcctgggcgggactggggagg

SEQ ID NO: 103 - DNA, 21 bp, Artificial Sequence
  Description of Artificial Sequence Synthetic oligonucleotide
gtccatcccatgcaggctcac

SEQ ID NO: 104 - DNA, 21 bp, Artificial Sequence
  Description of Artificial Sequence Synthetic oligonucleotide
gcggagagagaagtattagag

SEQ ID NO: 105 - DNA, 21 bp, Artificial Sequence
  Description of Artificial Sequence Synthetic oligonucleotide
gctttattgaggcttaagcag

SEQ ID NO: 106 - DNA, 20 bp, Artificial Sequence
  Description of Artificial Sequence Synthetic oligonucleotide
gcccgtctgttgtgtgactc

SEQ ID NO: 107 - protein, 1442 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGAPKKKRKVGSSGSPKKKRKVGSDALDDFDLDMLGSDALDDFDLDMLGSDALDDFDLDMLGSDALDDFDLDML

SEQ ID NO: 108 - protein, 473 aa, Artificial Sequence
  Description of Artificial Sequence Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 109 - DNA, 24 bp, Artificial Sequence
  Description of Artificial Sequence Synthetic oligonucleotide
caccgattggcagaactacacacc

SEQ ID NO: 110 - DNA, 24 bp, Artificial Sequence
  Description of Artificial Sequence Synthetic oligonucleotide
aaacggtgtgtagttctgccaatc

SEQ ID NO: 111 - DNA, 24 bp, Artificial Sequence
  Description of Artificial Sequence Synthetic oligonucleotide
caccgcgtggcctgggcgggactg

SEQ ID NO: 112 - DNA, 24 bp, Artificial Sequence
  Description of Artificial Sequence Synthetic oligonucleotide
aaaccagtcccgcccaggccacgc

SEQ ID NO: 113 - DNA, 27 bp, Artificial Sequence
  Description of Artificial Sequence Synthetic oligonucleotide
caccgatctgtggatctaccacacaca

SEQ ID NO: 114 - DNA, 27 bp, Artificial Sequence
  Description of Artificial Sequence Synthetic oligonucleotide
aaactgtgtgtggtagatccacagatc

SEQ ID NO: 115 - DNA, 27 bp, Artificial Sequence
  Description of Artificial Sequence Synthetic oligonucleotide
caccgctgcttatatgcagcatctgag

SEQ ID NO: 116 - DNA, 27 bp, Artificial Sequence
  Description of Artificial Sequence Synthetic oligonucleotide
aaacctcagatgctgcatataagcagc

SEQ ID NO: 117 - DNA, 25 bp, Artificial Sequence
  Description of Artificial Sequence Synthetic oligonucleotide
caccgtgtggtagatccacagatca

SEQ ID NO: 118 - DNA, 25 bp, Artificial Sequence
  Description of Artificial Sequence Synthetic oligonucleotide
aaactgatctgtggatctaccacac

SEQ ID NO: 119 - DNA, 25 bp, Artificial Sequence
  Description of Artificial Sequence Synthetic oligonucleotide
caccgcagggaagtagccttgtgtg

SEQ ID NO: 120 - DNA, 25 bp, Artificial Sequence
  Description of Artificial Sequence Synthetic oligonucleotide
aaaccacacaaggctacttccctgc

SEQ ID NO: 121 - DNA, 25 bp, Artificial Sequence
  Description of Artificial Sequence Synthetic oligonucleotide
caccgatcagatatccactgacctt

SEQ ID NO: 122 - DNA, 25 bp, Artificial Sequence
  Description of Artificial Sequence Synthetic oligonucleotide
aaacaaggtcagtggatatctgatc

SEQ ID NO: 123 - DNA, 25 bp, Artificial Sequence
  Description of Artificial Sequence Synthetic oligonucleotide
caccgcacactaatacttctccctc

SEQ ID NO: 124 - DNA, 25 bp, Artificial Sequence
  Description of Artificial Sequence Synthetic oligonucleotide
aaacgagggagaagtattagtgtgc

SEQ ID NO: 125 - DNA, 25 bp, Artificial Sequence
  Description of Artificial Sequence Synthetic oligonucleotide
caccgcctcctagcatttcgtcaca

SEQ ID NO: 126 - DNA, 25 bp, Artificial Sequence
  Description of Artificial Sequence Synthetic oligonucleotide
aaactgtgacgaaatgctaggaggc

SEQ ID NO: 127 - DNA, 25 bp, Artificial Sequence
  Description of Artificial Sequence Synthetic oligonucleotide
caccgcatggcccgagagctgcatc

SEQ ID NO: 128 - DNA, 25 bp, Artificial Sequence
  Description of Artificial Sequence Synthetic oligonucleotide
aaacgatgcagctctcgggccatgc

SEQ ID NO: 129 - DNA, 25 bp, Artificial Sequence
  Description of Artificial Sequence Synthetic oligonucleotide
caccgcagcagtctttgtagtactc

SEQ ID NO: 130 - DNA, 25 bp, Artificial Sequence
  Description of Artificial Sequence Synthetic oligonucleotide
aaacgagtactacaaagactgctgc

SEQ ID NO: 131 - DNA, 25 bp, Artificial Sequence
  Description of Artificial Sequence Synthetic oligonucleotide
caccgctgacatcgagctttctaca

SEQ ID NO: 132 - DNA, 25 bp, Artificial Sequence
  Description of Artificial Sequence Synthetic oligonucleotide
aaactgtagaaagctcgatgtcagc

SEQ ID NO: 133 - DNA, 25 bp, Artificial Sequence
  Description of Artificial Sequence Synthetic oligonucleotide
caccgtctacaagggactttccgct

SEQ ID NO: 134 - DNA, 25 bp, Artificial Sequence
  Description of Artificial Sequence Synthetic oligonucleotide
aaacagcggaaagtcccttgtagac

SEQ ID NO: 135 - DNA, 25 bp, Artificial Sequence
  Description of Artificial Sequence Synthetic oligonucleotide
caccgctttccgctggggactttcc

SEQ ID NO: 136 - DNA, 25 bp, Artificial Sequence
  Description of Artificial Sequence Synthetic oligonucleotide
aaacggaaagtccccagcggaaagc

SEQ ID NO: 137 - DNA, 25 bp, Artificial Sequence
  Description of Artificial Sequence Synthetic oligonucleotide
caccgctttattgaggcttaagcag

SEQ ID NO: 138 - DNA, 25 bp, Artificial Sequence
  Description of Artificial Sequence Synthetic oligonucleotide
aaacctgcttaagcctcaataaagc

SEQ ID NO: 139 - DNA, 24 bp, Artificial Sequence
  Description of Artificial Sequence Synthetic oligonucleotide
caccgcccgtctgttgtgtgactc

SEQ ID NO: 140 - DNA, 24 bp, Artificial Sequence
  Description of Artificial Sequence Synthetic oligonucleotide
aaacgagtcacacaacagacgggc

SEQ ID NO: 141 - DNA, 22 bp, Artificial Sequence
  Description of Artificial Sequence Synthetic primer
atggactatcatatgcttaccg

SEQ ID NO: 142 - DNA, 25 bp, Artificial Sequence
  Description of Artificial Sequence Synthetic primer
cagtgcaggggaaagaatagtagac

SEQ ID NO: 143 - DNA, 24 bp, Artificial Sequence
  Description of Artificial Sequence Synthetic primer
cagcggggctgctaaagcgcatgc

SEQ ID NO: 144 - DNA, 44 bp, Artificial Sequence
  Description of Artificial Sequence Synthetic primer
tgggatattgatgatctgtagtgctatggcgcgttcaacgctct

SEQ ID NO: 145 - DNA, 44 bp, Artificial Sequence
  Description of Artificial Sequence Synthetic primer
agagcgttgaacgcgccatagcactacagatcatcaatatccca

SEQ ID NO: 146 - DNA, 44 bp, Artificial Sequence
  Description of Artificial Sequence Synthetic primer
gcctatagagtacgagccataggtgggaataggagctttgttcc

SEQ ID NO: 147 - DNA, 44 bp, Artificial Sequence
  Description of Artificial Sequence Synthetic primer
ggaacaaagctcctattcccacctatggctcgtactctataggc

SEQ ID NO: 148 - DNA, 58 bp, Artificial Sequence
  Description of Artificial Sequence Synthetic primer
gcttggaaaggattttgctataagctagccaccatggaagatgccaagaacatcaaga

SEQ ID NO: 149 - DNA, 58 bp, Artificial Sequence
  Description of Artificial Sequence Synthetic primer
tcttgatgttcttggcatcttccatggtggctagcttatagcaaaatcctttccaagc

SEQ ID NO: 150 - DNA, 51 bp, Artificial Sequence
  Description of Artificial Sequence Synthetic primer
gccaagaagggcggcaagcctaggcctgccacgaacttctctctgttaaag

SEQ ID NO: 151 - DNA, 51 bp, Artificial Sequence
  Description of Artificial Sequence Synthetic primer
ctttaacagagagaagttcgtggcaggcctaggcttgccgcccttcttggc

SEQ ID NO: 152 - DNA, 51 bp, Artificial Sequence; identical to SEQ ID NO: 151
  Description of Artificial Sequence Synthetic primer